SEQUENCE LISTING (83 sequences)

SEQ ID NO: 1 - protein, 8 aa, synthetic construct
GDSITSGY

SEQ ID NO: 2 - protein, 7 aa, synthetic construct
ISYSGST

SEQ ID NO: 3 - protein, 10 aa, synthetic construct
ASHSHWYFDV

SEQ ID NO: 4 - protein, 6 aa, synthetic construct
QDISNY

SEQ ID NO: 5 - skipped

SEQ ID NO: 6 - protein, 9 aa, synthetic construct
QQGNTLPWT

SEQ ID NO: 7 - protein, 371 aa, Rattus norvegicus
MEAMAPRTLLLLLAAALAPTQTRAGSHSLRYFYTAVSRPGLGEPRFIAVGYVDDTEFVRFDSDAENPRMEPRARWMEREGPEYWEQQTRIAKEWEQIYRVDLRTLRGYYNQSEGGSHTIQEMYGCDVGSDGSLLRGYRQDAYDGRDYIALNEDLKTWTAADFAAQITRNKWERARYAERLRAYLEGTCVEWLSRYLELGKETLLRSDPPEAHVTLHPRPEGDVTLRCWALGFYPADITLTWQLNGEDLTQDMELVETRPAGDGTFQKWASVVVPLGKEQNYTCRVEHEGLPKPLSQRWEPSPSTDSNMETTVIYVILGAVAMIGAVAIIGAMVAVVRRRKRNTGGKGGDYAPAPGRDSSQSSDVSLPDCKA

SEQ ID NO: 8 - protein, 767 aa, Rattus norvegicus
MKTPWKVLLGLLGVAALVTIITVPVVLLNKDEAAADSRRTYTLADYLKNTFRVKSYSLRWVSDSEYLYKQENNILLFNAEHGNSSIFLENSTFEIFGDSISDYSVSPDRLFVLLEYNYVKQWRHSYTASYSIYDLNKRQLITEEKIPNNTQWITWSQEGHKLAYVWKNDIYVKIEPHLPSHRITSTGKENVIFNGINDWVYEEEIFGAYSALWWSPNGTFLAYAQFNDTGVPLIEYSFYSDESLQYPKTVWIPYPKAGAVNPTVKFFIVNTDSLSSTTTTIPMQITAPASVTTGDHYLCDVAWVSEDRISLQWLRRIQNYSVMAICDYDKTTLVWNCPTTQEHIETSATGWCGRFRPAEPHFTSDGSSFYKIVSDKDGYKHICQFQKDRKPEQVCTFITKGAWEVISIEALTSDYLYYISNEYKEMPGGRNLYKIQLTDHTNKKCLSCDLNPERCQYYSVSLSKEAKYYQLGCRGPGLPLYTLHRSTDQKELRVLEDNSALDKMLQDVQMPSKKLDFIVLNETRFWYQMILPPHFDKSKKYPLLIDVYAGPCSQKADAAFRLNWATYLASTENIIVASFDGRGSGYQGDKIMHAINKRLGTLEVEDQIEAARQFLKMGFVDSKRVAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDAGVDFQAMWYTDEDHGIASSTAHQHIYSHMSHFLQQCFSLR

SEQ ID NO: 9 - protein, 767 aa, Rattus norvegicus
MKTPWKVLLGLLGVAALVTIITVPVVLLNKDEAAADSARTYTLADYLKNTFRVKSYSLRWVSDSEYLYKQENNILLFNAEHGNSSIFLENSTFEIFGDSISDYSVSPDRLFVLLEYNYVKQWRHSYTASYSIYDLNKRQLITEEKIPNNTQWITWSQEGHKLAYVWKNDIYVKIEPHLPSHRITSTGKENVIFNGINDWVYEEEIFGAYSALWWSPNGTFLAYAQFNDTGVPLIEYSFYSDESLQYPKTVWIPYPKAGAVNPTVKFFIVNTDSLSSTTTTIPMQITAPASVTTGDHYLCDVAWVSEDRISLQWLRRIQNYSVMAICDYDKTTLVWNCPTTQEHIETSATGWCGRFRPAEPHFTSDGSSFYKIVSDKDGYKHICQFQKDRKPEQVCTFITKGAWEVISIEALTSDYLYYISNEYKEMPGGRNLYKIQLTDHTNKKCLSCDLNPERCQYYSVSLSKEAKYYQLGCRGPGLPLYTLHRSTDQKELRVLEDNSALDKMLQDVQMPSKKLDFIVLNETRFWYQMILPPHFDKSKKYPLLIDVYAGPCSQKADAAFRLNWATYLASTENIIVASFDGRGSGYQGDKIMHAINKRLGTLEVEDQIEAARQFLKMGFVDSKRVAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDAGVDFQAMWYTDEDHGIASSTAHQHIYSHMSHFLQQCFSLR

SEQ ID NO: 10 - protein, 355 aa, Rattus norvegicus
MTAAPLTPDPTHPRRRRKSYTFFSLGIYAEALLFLLSSLSDACEPPPPFEAMELKDKPKPHYAIGEIIEYTCKKGYLYLSPYPMTAICQPNHTWVPISDHGCIKVQCTMLQDPSFGKVHYIDGRFSWGARVKYTCMNGYYMVGMSVLQCELNGNGDAFWNGHPPSCKKVYCLPPPKIKNGTHTFTDIKVFKYHEAVIYSCDPNPGPDKFSLVGPSMLFCAGHNTWSSDPPECKVVKCPFPVLQNGRQISRTEKKFSYQALVLFQCLEGFYMEGSSMVVCGAKSSWEPSIPQCLKGPKPHSTKPPVYSESGYPSPREGIFGQEFDAWIIALIVVTSVVGVIVICLIILRCSEYRKK

SEQ ID NO: 11 - protein, 761 aa, Rattus norvegicus
MMDQARSAFSNLFGGEPLSYTRFSLARQVDGDNSHVEMKLAADEEENADSNMKASVRKPKRFNGRLCFATIAVVIFFLIGFMIGYLGYCKRVEQKEECVRLAEAEEADKSENDETEYVPKSSRLFWADLKTLLSEKLNSIEFTDIIKQLSQNTYTPREAGSQKDENLAYYIENLFHDFKFSKVWRDEHYVKIQVKNSVSQNLVTINSGSNIDPVEAPEGYVAFSKAGEVTGKLVHANFGTKKDFEELNYSVNGSLVIVRAGKITFAEKVANAQSFNAIGVLIYMDRNTFPVVEADLQFFGHAHLGTGDPYTPGFPSFNHTQFPPSQSSGLPSIPVQTISRAAAEKLFKNMEGNCPPSWNIDSSCKLELSQNQNVKLTVNNVLKETRILNIFGVIKGYEEPDRYIVVGAQRDAWGPGVAKSSVGTGLLLKLAQVFSDMISKDGFRPSRSIIFASWTAGDYGAVGATEWLEGYLSSLHLKAFTYINLDKVVLGTSNFKVSASPLLYTLMGKIMQDVKHPIDGKYLYRDSNWISKIEELSLDNAAFPFLAYSGIPAVSFCFCEDEDYPYLGTKLDTYEILIQKVPQLNQMVRTAAEVAGQFIIKLTHDIELTLDYEMYNSKLLSFMKDLNQFKADIKDMGLSLQWLYSARGDYFRATSRLTTDFHNAEKTNRFVMREINDRIMKVEYHFLSPYVSPRESPFRHIFWGSGSHTLSALVENLRLRQKNITAFNETLFRNQLALATWTIQGVANALSGDIWNIDNEF

SEQ ID NO: 12 - protein, 280 aa, Rattus norvegicus
MDDQRDLISNHEQLPILGQRARAPESNCNRGVLYTSVSVLVALLLAGQATTAYFLYQQQGRLDKLTVTSQNLQLENLRMKLPKSAKPVSPMRMATPLLMRPLSMDNMLQAPVKNVTKYGNMTQDHVMHLLTKSGPVNYPQLKGSFPENLKHLKNSMNGLDWKVFESWMKQWLLFEMSKNSLEEKQPTQTPPKVLTKCQEEVSHIPDVHPGAFRPKCDENGNYMPLQCHGSTGYCWCVFPNGTEVPHTKSRGRHNCSEPLDMEDPSSGLGVTKQDMGQMFL

SEQ ID NO: 13 - protein, 216 aa, Rattus norvegicus
MDDQRDLISNHEQLPILGQRARAPESNCNRGVLYTSVSVLVALLLAGQATTAYFLYQQQGRLDKLTVTSQNLQLENLRMKLPKSAKPVSPMRMATPLLMRPLSMDNMLQAPVKNVTKYGNMTQDHVMHLLTKSGPVNYPQLKGSFPENLKHLKNSMNGLDWKVFESWMKQWLLFEMSKNSLEEKQPTQTPPKEPLDMEDPSSGLGVTKQDMGQMFL

SEQ ID NO: 14 - DNA, 1323 bp, synthetic construct
  source 1..1323: mol_type = other DNA
gaggtgcagcttcaggagtcaggacctagcctcgtgaaaccttctcagactctgtccctcacctgttctgtcactggcgactccatcaccagtggttactggaactggatccggaaattcccagggaataaacttgagtacatggggtacataagctacagtggtagcacttactacaatccatctctcaaaagtcgaatctccatcactcgagacacatccaagaaccagtactacctgcagttgaattctgtgactactgaggacacagccacatattactgtgcaagtcatagccactggtacttcgatgtctggggcgcagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 15 - protein, 440 aa, synthetic construct
EVQLQESGPSLVKPSQTLSLTCSVTGDSITSGYWNWIRKFPGNKLEYMGYISYSGSTYYNPSLKSRISITRDTSKNQYYLQLNSVTTEDTATYYCASHSHWYFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 16 - protein, 116 aa, synthetic construct
EVQLQESGPSLVKPSQTLSLTCSVTGDSITSGYWNWIRKFPGNKLEYMGYISYSGSTYYNPSLKSRISITRDTSKNQYYLQLNSVTTEDTATYYCASHSHWYFDVWGAGTTVTVSS

SEQ ID NO: 17 - DNA, 645 bp, synthetic construct
  source 1..645: mol_type = other DNA
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 18 - protein, 214 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 20 - protein, 25 aa, synthetic construct
EVQLQESGPSLVKPSQTLSLTCSVT

SEQ ID NO: 21 - protein, 25 aa, synthetic construct
EVQLQESGPSLVKPSQTLSLTCSVT

SEQ ID NO: 22 - protein, 30 aa, synthetic construct
EVQLQESGPSLVKPSQTLSLTCSVTGDSIT

SEQ ID NO: 23 - protein, 29 aa, synthetic construct
EVQLQESGPSLVKPSQTLSLTCSVTGDSI

SEQ ID NO: 24 - protein, 25 aa, synthetic construct
EVQLQESGPSLVKPSQTLSLTCSVT

SEQ ID NO: 25 - protein, 7 aa, synthetic construct
GDSITSG

SEQ ID NO: 26 - protein, 10 aa, synthetic construct
GDSITSGYWN

SEQ ID NO: 27 - protein, 5 aa, synthetic construct
SGYWN

SEQ ID NO: 28 - protein, 6 aa, synthetic construct
TSGYWN

SEQ ID NO: 29 - protein, 19 aa, synthetic construct
YWNWIRKFPGNKLEYMGYI

SEQ ID NO: 30 - protein, 14 aa, synthetic construct
WIRKFPGNKLEYMG

SEQ ID NO: 31 - protein, 14 aa, synthetic construct
WIRKFPGNKLEYMG

SEQ ID NO: 32 - protein, 11 aa, synthetic construct
WIRKFPGNKLE

SEQ ID NO: 33 - protein, 17 aa, synthetic construct
WNWIRKFPGNKLEYMGY

SEQ ID NO: 34 - protein, 5 aa, synthetic construct
SYSGS

SEQ ID NO: 35 - protein, 9 aa, synthetic construct
YISYSGSTY

SEQ ID NO: 36 - protein, 16 aa, synthetic construct
YISYSGSTYYNPSLKS

SEQ ID NO: 37 - protein, 12 aa, synthetic construct
YMGYISYSGSTY

SEQ ID NO: 38 - protein, 41 aa, synthetic construct
TYYNPSLKSRISITRDTSKNQYYLQLNSVTTEDTATYYCAS

SEQ ID NO: 39 - protein, 39 aa, synthetic construct
YNPSLKSRISITRDTSKNQYYLQLNSVTTEDTATYYCAS

SEQ ID NO: 40 - protein, 32 aa, synthetic construct
RISITRDTSKNQYYLQLNSVTTEDTATYYCAS

SEQ ID NO: 41 - protein, 37 aa, synthetic construct
YNPSLKSRISITRDTSKNQYYLQLNSVTTEDTATYYC

SEQ ID NO: 42 - protein, 38 aa, synthetic construct
YYNPSLKSRISITRDTSKNQYYLQLNSVTTEDTATYYC

SEQ ID NO: 43 - protein, 8 aa, synthetic construct
HSHWYFDV

SEQ ID NO: 44 - protein, 8 aa, synthetic construct
HSHWYFDV

SEQ ID NO: 45 - protein, 8 aa, synthetic construct
HSHWYFDV

SEQ ID NO: 46 - protein, 9 aa, synthetic construct
ASHSHWYFD

SEQ ID NO: 47 - protein, 11 aa, synthetic construct
WGAGTTVTVSS

SEQ ID NO: 48 - protein, 11 aa, synthetic construct
WGAGTTVTVSS

SEQ ID NO: 49 - protein, 11 aa, synthetic construct
WGAGTTVTVSS

SEQ ID NO: 50 - protein, 12 aa, synthetic construct
VWGAGTTVTVSS

SEQ ID NO: 51 - protein, 11 aa, synthetic construct
WGAGTTVTVSS

SEQ ID NO: 52 - protein, 23 aa, synthetic construct
DIQMTQTTSSLSASLGDRVTISC

SEQ ID NO: 53 - protein, 23 aa, synthetic construct
DIQMTQTTSSLSASLGDRVTISC

SEQ ID NO: 54 - protein, 23 aa, synthetic construct
DIQMTQTTSSLSASLGDRVTISC

SEQ ID NO: 55 - protein, 29 aa, synthetic construct
DIQMTQTTSSLSASLGDRVTISCRASQDI

SEQ ID NO: 56 - protein, 26 aa, synthetic construct
DIQMTQTTSSLSASLGDRVTISCRAS

SEQ ID NO: 57 - protein, 11 aa, synthetic construct
RASQDISNYLN

SEQ ID NO: 58 - protein, 11 aa, synthetic construct
RASQDISNYLN

SEQ ID NO: 59 - protein, 11 aa, synthetic construct
RASQDISNYLN

SEQ ID NO: 60 - protein, 7 aa, synthetic construct
SNYLNWY

SEQ ID NO: 61 - protein, 15 aa, synthetic construct
WYQQKPDGTVKLLIY

SEQ ID NO: 62 - protein, 15 aa, synthetic construct
WYQQKPDGTVKLLIY

SEQ ID NO: 63 - protein, 15 aa, synthetic construct
WYQQKPDGTVKLLIY

SEQ ID NO: 64 - protein, 9 aa, synthetic construct
QQKPDGTVK

SEQ ID NO: 65 - protein, 17 aa, synthetic construct
LNWYQQKPDGTVKLLIY

SEQ ID NO: 66 - protein, 7 aa, synthetic construct
YTSRLHS

SEQ ID NO: 67 - protein, 7 aa, synthetic construct
YTSRLHS

SEQ ID NO: 68 - protein, 7 aa, synthetic construct
YTSRLHS

SEQ ID NO: 69 - protein, 10 aa, synthetic construct
LLIYYTSRLH

SEQ ID NO: 70 - protein, 32 aa, synthetic construct
GVPSRFSGSGSGTDYSLTISNLEQEDIATYFC

SEQ ID NO: 71 - protein, 32 aa, synthetic construct; identical to SEQ ID NO: 70

SEQ ID NO: 72 - protein, 32 aa, synthetic construct; identical to SEQ ID NO: 70

SEQ ID NO: 73 - protein, 33 aa, synthetic construct
SGVPSRFSGSGSGTDYSLTISNLEQEDIATYFC

SEQ ID NO: 74 - protein, 37 aa, synthetic construct
SRLHSGVPSRFSGSGSGTDYSLTISNLEQEDIATYFC

SEQ ID NO: 75 - protein, 9 aa, synthetic construct
QQGNTLPWT

SEQ ID NO: 76 - protein, 9 aa, synthetic construct
QQGNTLPWT

SEQ ID NO: 77 - protein, 9 aa, synthetic construct
QQGNTLPWT

SEQ ID NO: 78 - protein, 8 aa, synthetic construct
QQGNTLPW

SEQ ID NO: 79 - protein, 12 aa, synthetic construct
FGGGTKLEIKRA

SEQ ID NO: 80 - protein, 12 aa, synthetic construct
FGGGTKLEIKRA

SEQ ID NO: 81 - protein, 12 aa, synthetic construct
FGGGTKLEIKRA

SEQ ID NO: 82 - protein, 13 aa, synthetic construct
TFGGGTKLEIKRA

SEQ ID NO: 83 - protein, 12 aa, synthetic construct
FGGGTKLEIKRA

(19) United States
(12) Patent Application Publication
(10) Pub. No.: US 2025/0009808 A1
(43) Pub. Date: Jan. 9, 2025

(54) Methods of Producing and Using Human Hepatocytes and Related Compositions

(71) Applicant: Cytotheryx, Inc., Rochester, MN (US)

(72) Inventors: Kevin Keys, Rochester, MN (US); Allyson Merrell, Rochester, MN (US); Yambazi Banda, Rochester, MN (US); Otto Guedelhoefer, Rochester, MN (US); Kenneth Dorko, Rochester, MN (US); Raymond Hickey, Rochester, MN (US); Michael Holmes, Rochester, MN (US); Tin Mao, Rochester, MN (US); Alan Mendoza, Rochester, MN (US); Leslie Stewart, Rochester, MN (US); Elizabeth Wilson, Rochester, MN (US); Rafal Witek, Rochester, MN (US)

(21) Appl. No.: 18/709,374

(22) PCT Filed: Nov. 11, 2022
(86) PCT No.: PCT/US2022/079747

§ 371 Date: May 10, 2024

Related U.S. Application Data
(60) Provisional application No. 63/332,022, filed on Apr. 18, 2022.
(60) Provisional application No. 63/332,020, filed on Apr. 18, 2022.
(60) Provisional application No. 63/278,854, filed on Nov. 12, 2021.

(51) Int. Cl. A61K 35/407; C07K 16/28; C12N 5/071

(52) U.S. Cl. CPC A61K 35/407; C07K 16/2833; C12N 5/0671; C12N 2501/50

(57) ABSTRACT

Provided are isolated expanded human hepatocytes and methods of producing isolated expanded human hepatocytes. In certain embodiments, the methods comprise introducing human hepatocytes into the liver of a non-human in vivo bioreactor, expanding the human hepatocytes in the liver of the non-human in vivo bioreactor, and collecting hepatocytes from the liver of the non-human in vivo bioreactor. The collected hepatocytes comprise a xenomixture of expanded human hepatocytes and non-human in vivo bioreactor cells, including hepatocytes endogenous to the in vivo bioreactor. Such methods may further comprise subjecting the xenomixture to centrifugal elutriation under conditions sufficient to produce an elutriation fraction enriched for the expanded human hepatocytes, and/or removing non-human in vivo bioreactor cells from the elutriation fraction via a negative selection process. Also provided are isolated expanded human hepatocytes produced according to such methods. Centrifugal elutriation- and negative selection-based methods of enriching for human hepatocytes in a xenomixture, and certain compositions useful in such methods, are also provided.

### INTRODUCTION

Orthotopic liver transplantation (OLT) is the current gold-standard therapy for end-stage liver disease, acute liver failure, and liver-based metabolic disorders, and is the only intervention with proven clinical benefits and long-lasting effects (lansante et al (2018) Pediatric Res. 83(1):232-240). However, there are not nearly enough suitable donor organs for the number of patients that could benefit from OLT. For example, UNOS reported record-breaking numbers of liver transplants in recent years, with 8,906 liver transplants performed in the United States in 2020; however, these numbers remain a fraction of the patients awaiting a liver (e.g., >25,000 in 2020) and do not help the many with conditions which are debilitating but considered not urgent enough to justify OLT, those excluded from transplant, or the about 90 per month who die while waiting (Kwong et al. (2022) Am J Transplant 22 Suppl 2:204-309). This also does not include the pediatric liver transplant waiting list, which added 616 new registrants in 2020 alone. Of the pediatric subjects previously on the list nearly a third (30.3%) of patients had already waited 2 years or more, with 10% having waited greater than 5 years, for a transplant. OLT numbers and the liver waitlists represent only a snapshot of the burden of acute and chronic liver failure, which affects millions of patients worldwide and has an average survival time of about two years (GBD 2017, Global Health Metrics 392(10159):1789-1858).

Human hepatocyte transplant may ameliorate many burdens of various liver diseases, including acute and chronic liver failure. However, a general lack of enough human hepatocytes that are readily available and suitable for transplantation remains a significant obstacle to the widespread adoption of human hepatocyte transplantation as a “go to” therapeutic for liver diseases whether or not such diseases are candidates for OLT (lansante et al. supra).

Human hepatocytes are also widely used by the pharmaceutical industry during preclinical drug development. Indeed, their use is mandated by the FDA for this purpose. For drug metabolism and other studies and purposes, hepatocytes are typically isolated from cadaveric organ donors and shipped to the location where testing will be performed. The condition (viability and state of differentiation) of hepatocytes from cadaveric sources is highly variable and many cell preparations are of marginal quality. Human hepatocytes are also necessary for studies in the fields of microbiology and virology. Many human viruses, such as viruses that cause hepatitis, cannot infect and/or replicate in any other cell type.

The availability of high-quality human hepatocytes is further hampered by the fact that they cannot be significantly expanded in tissue culture (Runge et al. (2000) Biochem. Biophys. Res. Commun. 274:1-3; Cascio et al. (2001) Organs 25:529-538). After plating, the cells may survive but do not divide and/or rapidly lose hepatocyte characteristics. Hepatocytes from readily available mammalian species, such as the mouse, are not suitable for human drug testing because they have a different complement of metabolic enzymes and respond differently in induction studies. Such hepatocytes are also not suitable for therapeutic hepatocyte transplantation due to xeno-rejection and species-to-species differences in liver cell metabolism and liver-produced proteins. Immortal human liver cells (hepatomas) or fetal hepatoblasts are also not an adequate replacement for fully differentiated adult liver cells.

Human hepatocytes cannot be expanded significantly in culture. Hepatocytes derived from stem cells in culture are immature and generally lack full functionality. Therefore, hepatocytes in use today are derived from human donors, either cadaveric or surgical specimens, which significantly limits hepatocyte availability.

### SUMMARY

Provided are isolated expanded human hepatocytes and methods of producing isolated expanded human hepatocytes. In certain embodiments, the methods comprise introducing human hepatocytes into the liver of a non-human in vivo bioreactor, expanding the human hepatocytes in the liver of the non-human in vivo bioreactor, and collecting hepatocytes from the liver of the non-human in vivo bioreactor. The collected hepatocytes comprise a xenomixture of expanded human hepatocytes and non-human in vivo bioreactor cells, including hepatocytes endogenous to the in vivo bioreactor. Such methods may further comprise subjecting the xenomixture to centrifugal elutriation under conditions sufficient to produce an elutriation fraction enriched for the expanded human hepatocytes, and/or removing non-human in vivo bioreactor cells from the elutriation fraction via a negative selection process. Also provided are isolated expanded human hepatocytes produced according to such methods. Centrifugal elutriation- and negative selection-based methods of enriching for human hepatocytes in a xenomixture, and certain compositions useful in such methods, are also provided.

### BRIEF DESCRIPTION OF THE FIGURES

FIG. 1: Data showing the percent of total rat cells bound by antibodies to each of a subset of rat antigens evaluated.

FIG. 2: Data showing the percent of total rat cells bound by three different anti-RT1A monoclonal antibodies.

FIG. 3A-3B: Data showing the recovery of human cells via anti-RT1A-based negative selection evaluated using defined xenomixtures containing various ratios of rat to human cells (FIG. 3A). FIG. 38 re-displays the data as percent of theoretical recovery from the human-cell-containing xenomixtures.

FIG. 4: A non-limiting example overview of the isolation and enrichment workflow for processing of human hepatocytes FIG. 5A-5H: Data showing anti-RT1A-based purification and enrichment achieved at various points during non-optimized trial runs.

FIG. 6A-6E: Data showing the functional characteristics of human hepatocytes isolated using a Percoll-based process (P) or an elutriation-based process (E).

FIG. 7: Data showing the relative expression levels of mRNAs encoding inflammatory cytokines, interleukin 1-beta (IL-1beta), interleukin 6 (IL-6), tumor necrosis factor alpha (TNFalpha), and tumor necrosis factor beta (TNFbeta), in fractions of human hepatocyte lots processed using either Percoll density gradient centrifugation (“P”) or elutriation (“E”).

FIG. 8: Data showing the percent recovery of the total number of pig cells using two candidate monoclonal antibodies specific for swine MHC class I antigen, also referred to as swine leukocyte antigen 1 (SLA-1).

FIG. 9: Flow cytometric data testing the anti-SLA-1 monoclonal antibodies for cross-reactivity with human cells.

FIG. 10: Data showing the input ratio of human to pig cells (left bar of each pair, black and gray representing pig and human, respectively) and the percent of the total input cells obtained in the flowthrough (right bar of each pair) using a candidate anti-SLA-1 monoclonal antibody.

FIG. 11: Magnetic selection data using magnetic bead conjugated secondary antibody that binds to the anti-rat RT1A class I histocompatibility antigen antibody.

FIG. 12A-12B: Global gene expression pattern analysis produced from single-cell RNA-Seq analysis of expanded hepatocytes from FRG rat bioreactors processed and isolated according to the methods described herein, compared to healthy unexpanded cadaveric hepatocytes, rendered as a Uniform Manifold Approximation and Projection (UMAP) plot (FIG. 12A) and a principal component analysis (PCA) (FIG. 12B).

FIG. 13: Levels of human albumin (hAlb, micrograms/milliliter) as measured by ELISA in blood samples collected from mice transplanted with cells processed and isolated according to the methods described herein, compared to mice transplanted with unexpanded cadaveric donor primary human hepatocytes.

FIG. 14: Whole blood hAlb concentration data demonstrating that transplanted huFRG human hepatocytes were functional and capable of engrafting and expanding in cDNA-uPA/SCID recipient mice.

FIG. 15: Comparative ammonia detoxification data demonstrating engraftment, proliferation, expansion, and substantial function of transplanted huFRG cells in vivo.

FIG. 16: PCA plot generated from bulk RNAseq data of human hepatocytes expanded and processed as described herein, in-house isolated primary human hepatocytes, and commercial primary human hepatocytes and including available datasets from primary human hepatocytes (PHH) and hepatocyte like cells (HLCs).

FIG. 17: Dendrogram plot from bulk RNAseq gene expression data depicting the relatedness of different populations of cells, including human hepatocytes expanded and processed as described herein, PHH from various sources, and HLCs.

### DETAILED DESCRIPTION

Before the methods and compositions of the present disclosure are described in greater detail, it is to be understood that the methods and compositions are not limited to particular embodiments described, as such may, of course, vary. It is also to be understood that the terminology used herein is for the purpose of describing particular embodiments only, and is not intended to be limiting, since the scope of the methods and compositions will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions. The upper and lower limits of these smaller ranges may independently be included in the smaller ranges and are also encompassed within the methods and compositions, subject to any specifically excluded limit in the stated range. Where the stated range includes one or both of the limits, ranges excluding either or both of those included limits are also included in the methods and compositions.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and compositions belong. Although any methods and compositions similar or equivalent to those described herein can also be used in the practice or testing of the methods and compositions, representative illustrative methods and compositions are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and compositions are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and compositions, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and compositions and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Producing Human Hepatocytes

Aspects of the present disclosure include methods of producing isolated expanded human hepatocytes. In certain embodiments, the methods may include collecting expanded human hepatocytes from a xenomixture of cells obtained from an in vivo bioreactor liver. In some instances, the methods comprise introducing human hepatocytes into the liver of a non-human in vivo bioreactor, expanding the human hepatocytes in the liver of the non-human in vivo bioreactor, and collecting hepatocytes from the liver of the non-human in vivo bioreactor, where the collected hepatocytes comprise a xenomixture of expanded human hepatocytes and non-human in vivo bioreactor cells, including e.g., in vivo bioreactor hepatocytes, in vivo bioreactor non-parenchymal cells (e.g., liver sinusoidal endothelial cells (LSEC), Kupffer cells, lymphocytes, biliary cells, and hepatic stellate cells (HSCs)), and the like. Such methods may further comprise subjecting the xenomixture to centrifugal elutriation under conditions sufficient to produce an elutriation fraction enriched for the expanded human hepatocytes, and/or removing non-human in vivo bioreactor cells from the elutriation fraction via a negative selection process to produce isolated expanded human hepatocytes.

The hepatocyte production methods of the present disclosure are based in part on a number of surprising findings demonstrated herein. Such findings include, but are not limited to, the finding that human hepatocytes may be expanded in an in vivo bioreactor and separated effectively from non-human in vivo bioreactor hepatocytes, that effective separation can be performed via elutriation, that negative selection procedures may also be employed in effectively separating expanded human hepatocytes from xenomixtures, that the expanded human hepatocytes have functional characteristics comparable, or superior, to the unexpanded human hepatocytes prior to introduction into the bioreactor, that the expanded human hepatocytes collected according to the procedures described herein may be distinct, e.g., in terms of gene expression, from the corresponding hepatocytes prior to in vivo bioreactor expansion and collection, and that the human hepatocytes processed using an elutriation-based procedure as described herein are, in some instances, superior in function to hepatocytes from the same donor liver processed using density sedimentation and/or density centrifugation-based (e.g., Percoll-based) procedures. For example, human hepatocytes processed according to the methods of the present disclosure exhibit improved plateability, increased attachment efficiency, better ammonia detoxification, increased human albumin production, increased A1AT production, and higher CYP3A4 activity as compared to corresponding hepatocytes isolated from the same donor liver using other methods, such as e.g., a different method that primarily employs a Percoll-based procedure in place of elutriation. In addition, populations of isolated and expanded human hepatocytes produced using the methods described herein surprisingly demonstrated in vivo characteristics (e.g., engraftment, expansion, human albumin production, etc.) comparable, or superior, to cadaveric primary human hepatocytes (PHH).

Moreover, the methods of the present disclosure reduce the presence of undesirable immune cells and inflammatory cytokines as compared to fractions processed using conventional methods, such as, e.g., density gradient centrifugation using Percoll. Also demonstrated herein is the surprising effectiveness of a negative selection process for the enrichment and further purification of human hepatocytes from xenomixtures.

The term “Percoll”, as used herein, generally refers to colloidal suspensions of silica particles, in water, which have been coated with polyvinylpyrrolidone to provide a density gradient media of low-viscosity that is non-toxic and suitable for density gradient centrifugation of cells, viruses and subcellular particles (see e.g., Pertoft et al. (1978) Analytical Biochemistry. 88 (1):271-282). As will be readily understood, in some instances, other suitable density gradient mediums may be substituted. Non-limiting examples of useful density gradient mediums include iodixanol-based density gradients, such as e.g., Optiprep and derivatives thereof, and polysaccharide-based density gradients, such as highly-branched, hydrophilic polymers such as e.g., Ficoll and derivatives thereof. Where employed, in some instances, density gradient medium having a density ranging from 1.01 to 1.05 g/mL, 1.02 to 1.05 g/mL, 1.03 to 1.05 g/mL, 1.04 to 1.05 g/mL, 1.01 to 1.04 g/mL, 1.02 to 1.04 g/mL, 1.03 to 1.04 g/mL, 1.01 to 1.02 g/mL, 1.01 to 1.03 g/mL, or 1.02 to 1.03 g/mL or may be used, e.g., at a concentration ranging from, e.g., 15% to 35%, 15% to 30%, 20% to 35%, 20% to 30%, 25% to 35%, 25% to 30%, 20% to 25%, 20% to 23%, 21% to 24%, 22% to 25%, 23% to 26%, 23% to 27% 24% to 27%, or 25% to 28% of the density gradient medium (e.g., Percoll).

The term “hepatocyte” refers to a type of cell that generally, by various estimates, makes up 60-70% or 70-80% of the cytoplasmic mass of the liver. Hepatocytes are involved in protein synthesis, protein storage and transformation of carbohydrates, synthesis of cholesterol, bile salts and phospholipids, and detoxification, modification and excretion of exogenous and endogenous substances. The hepatocyte also initiates the formation and secretion of bile. Hepatocytes manufacture serum albumin, fibrinogen and the prothrombin group of clotting factors and are the main site for the synthesis of lipoproteins, ceruloplasmin, transferrin, complement and glycoproteins. In addition, hepatocytes have the ability to metabolize, detoxify, and inactivate exogenous compounds such as drugs and insecticides, and endogenous compounds such as steroids.

The terms “in vivo bioreactor” and “non-human in vivo bioreactor” and sometimes simply “bioreactor”, as used herein, generally refer to a living non-human animal, such as a non-human mammal (e.g., a rodent (e.g., a rat or a mouse), a pig, etc.), into which exogenous cells, such as PHH and/or other types of hepatocyte-generating cells (i.e., cells that produce hepatocytes such as hepatocytes and/or hepatocyte progenitors), are introduced for engraftment and expansion. Non-human in vivo bioreactors may be used to generate an expanded population of desired cells (which may include the introduced cells and/or their progeny), such as an expanded population of hepatocytes, generated from the introduced cells. Introduction of exogenous cells, such as PHH and/or other types of hepatocyte-generating cells, into the bioreactor will generally involve xenotransplantation and, as such, the transplanted exogenous cells may, in some instances, be referred to as a xenograft, e.g., human-to-rodent xenograft, human-to-mouse xenograft, human-to-rat xenograft, human-to-porcine xenograft, mouse-to-rat xenograft, rat-to-mouse xenograft, rodent-to-porcine xenograft, etc. In some instances, allotransplantation into a bioreactor may be performed, e.g., rodent-to-rodent, porcine-to-porcine, etc., allotransplantations. As such, human or non-human cells may be introduced into an in vivo bioreactor. However, in some instances, a method may be employed solely for the production of human cells in a non-human in vivo bioreactor and may exclude, e.g., the production of non-human cells. A non-human in vivo bioreactor may be configured, e.g., genetically and/or pharmacologically, to confer a selective advantage to introduced exogenous cells, such as introduced exogenous hepatocyte-generating cells, in order to promote engraftment and/or expansion thereof. Bioreactors may, in some instances, be configured to prevent rejection of introduced exogenous cells, including but not limited to e.g., through genetic and/or pharmacological immune suppression. As such, non-human in vivo bioreactors may be subjected to external manipulation, e.g., through modulation of the animal's environment and/or the administration of one or more agents, e.g., to promote engraftment, to prevent rejection, to prevent infection, to maintain health, etc.

The present methods of producing isolated expanded human hepatocytes may comprise introducing human hepatocytes into the liver of the non-human in vivo bioreactor. Human hepatocytes that find use in the methods, and other aspects, of the present disclosure include hepatocytes obtained from human donors, including cadaveric and live human donors. In some embodiments, the hepatocytes are primary human hepatocytes (PHH) isolated from screened cadaveric donors, including fresh PHH or cryopreserved PHH. Useful cadaveric liver tissues include whole liver and partial liver samples. Cadaveric livers, including whole or partial organs that are or are not suitable for OLT, may be obtained from donors or organ procurement agencies. In some instances, useful PHH may be obtained from live donor tissues, including essentially any hepatocyte-containing biological sample, such as but not limited to, e.g., resected liver tissue, liver biopsy tissue, and the like. Donors, including live and cadaveric, and/or donor tissues, may be screened, e.g., for certain criteria and, based on such screening, the donor, liver, and/or liver tissue may be deemed suitable or unsuitable for OLT and/or use in the herein described methods and/or compositions.

Criteria useful in assessing the suitableness of a donor, donor liver, or donor tissue include but are not limited to e.g., donor age (e.g., 80 years or younger, 70 years or younger, 60 years or younger, 55 years or younger, 50 years or younger, 45 years or younger, 40 years or younger, 35 years or younger, 30 years or younger, 25 years or younger, 20 years or younger, 18 years or younger, 16 years or younger, 14 years or younger, 12 years or younger, 10 years or younger, 8 years or younger, 6 years or younger, 4 years or younger, 3 years or younger, 2 years or younger, 1 year or younger, etc.), time deceased or time from withdrawal of support (e.g., 1 hr or less, 30 min or less, etc.), time to transplant or other use (e.g., 12 hr or less, 10 hr or less, 8 hr or less, etc.), appearance, fat content, donor medical history (e.g., infection history (e.g., HCV, HBV, etc.), medication history, etc.), liver function (e.g., as assessed by liver function tests), and the like. In some instances, assessment criteria for donor, liver, or liver portion suitable for use in the herein described methods may be equally, more, or less stringent than criteria commonly employed for evaluation of donors, livers, and liver portions for OLT.

For example, in some instances, a liver tissue unsuitable for direct use as a therapeutic may provide hepatocytes that, when processed according to the methods as described herein, may produce a population of isolated expanded human hepatocytes useful for administration to a subject in need thereof. For example, in some instances a liver unsuitable for OLT may provide hepatocytes, that when processed according to the methods as described herein, produce a population of isolated expanded human hepatocytes useful for administration to a subject in need thereof. In some instances, the methods described herein may employ PHH that are suitable for transplantation or obtained from liver tissue or a whole liver that is suitable for OLT.

In certain embodiments, the human hepatocytes introduced into the liver of the non-human in vivo bioreactor were obtained by perfusion. For example, human liver (including, e.g., whole liver, partial liver, obtained liver tissue, etc.) may be perfused to obtain a cell population that includes human hepatocytes, such as PHH. Suitable methods of perfusion include, but are not limited to, enzymatic and/or chemical means, the method described in the Experimental section herein, and the like. Accordingly, in some instances, cell populations may be prepared from primary hepatic cell preparations, including e.g., cell populations prepared from human liver that include PHH, where such populations may or may not include hepatic cells other than hepatocytes. In certain embodiments, the hepatocytes are PHH isolated from screened cadaveric donors, including fresh PHH or cryopreserved PHH. In some instances, PHH of a cell population have undergone no or a minimal number of cell cycles/divisions since isolation from a liver, including but not limited to e.g., 1 or less, 2 or less, 3 or less, 4 or less, 5 or less, 6 or less, 7 or less, 8 or less, 9 or less, 10 cycles/divisions or less.

Useful hepatocytes, e.g., for introduction into the liver of the non-human in vivo bioreactor, include those obtained from commercial sources. Useful sources of commercially available hepatocytes include but are not limited to e.g., Thermo Fisher Scientific, Inc.; Corning, Inc.; LifeNet Health LifeSciences; BioIVT, LLC (inc. XenoTech, LLC); Discovery Life Science; AcceGen Biotechnology; and the like.

In some instances, cell populations may include, or may specifically exclude, hepatocyte progenitors. As used herein, the terms “hepatocyte progenitors” and “progenitors of hepatocytes” or the like, generally refer to cells from which hepatocytes are derived and/or cells that are differentiated into hepatocytes. In some instances, hepatocyte progenitors may be committed progenitors, meaning the progenitors will essentially only differentiate into hepatocytes. In some instances, hepatocyte progenitors may have varied potency and may be e.g., pluri-, multi-, or totipotent progenitors, including e.g., bi-potent progenitors. Hepatocyte progenitors may include or be derived from stem cells, induced pluripotent stem cells (iPSCs), embryonic stem (ES) cells, hepatocyte-like cells (HLCs), and the like. In some instances, hepatocyte progenitors may be derived from mature hepatocytes and/or other non-hepatocyte cells, e.g., through dedifferentiation of hepatocytes and/or transdifferentiation of other hepatic or non-hepatic cell types.

Hepatocytes obtained from the liver of an individual donor may be kept separate from the hepatocytes obtained from other individual donors or the hepatocytes of multiple individual donors (e.g., 2, 3, 4, 5, 6, 7, 8, 9, 10 or more) may be pooled together. Where employed, pooling may be performed at any convenient point in the process or before or during use, including but not limited to, e.g., during collection/harvest, following collection/harvest, during preparation for transplantation, before expansion, during expansion, following expansion, during collection/harvest from a bioreactor, following collection/harvest from a bioreactor, before enrichment, during enrichment, following enrichment, before isolation, during isolation, following isolation, before cryopreservation, during cryopreservation, following cryopreservation, during thawing, after thawing, before dose preparation, during dose preparation, after dose preparation, before administration, during administration (e.g., by administration of multiple separate aliquots to a single individual), etc. In some instances, no pooling takes place, including e.g., where a dose is prepared and/or a subject is administered expanded and isolated hepatocytes derived from a single human donor.

In some instances, pooling may include combining of multiple frozen aliquots of hepatocytes such that, e.g., when the frozen aliquots of cells are thawed together in a single container or vessel, the previously frozen cells are mixed together in a single composition. Useful methods of pooling frozen aliquots of cells include, but are not limited to e.g., those described in U.S. Pat. No. 9,642,355, the disclosure of which is herein incorporated by reference in its entirety.

Any suitable approach for introducing the human hepatocytes into the liver of the non-human in vivo bioreactor may be employed. According to some embodiments, introducing the human hepatocytes into the liver of the non-human in vivo bioreactor comprises delivering the human hepatocytes to the spleen of the non-human in vivo bioreactor. In one non-limiting example, the human hepatocytes may be introduced into the liver of the non-human in vivo bioreactor via splenic injection (e.g., laparotomy splenic injection or percutaneous splenic injection) as described in the Experimental section herein. In some instances, the human hepatocytes may be introduced into the liver of the non-human in vivo bioreactor via portal vein injection.

In some instances, prior to transplantation into an in vivo bioreactor, hepatocytes are subjected to methods for enhancing repopulation, engraftment, survival and/or expansion of human hepatocytes that involve contacting the hepatocytes ex vivo with compositions for enhanced repopulation, engraftment, survival and/or expansion of human hepatocytes that are transplanted into in vivo bioreactors, including where such methods include those described in US Pat. Pub No. 20210024885, the disclosure of which is incorporated herein by reference in its entirety. For example, in some instances, a method of the present disclosure may include an ex vivo manipulation that comprises culturing hepatocytes or other hepatocyte-generating cells with at least one agent that promotes growth, regeneration, survival and/or engraftment of hepatocytes in an in vivo bioreactor, including e.g., where the at least one agent is an agonist, such as an antibody, a small molecule, or a nucleic acid, including where the agonist is a hepatocyte growth factor receptor (c-MET) agonist or an epidermal growth factor (EGFR) agonist.

The hepatocyte production methods of the present disclosure comprise expanding the human hepatocytes in the liver of the non-human in vivo bioreactor. When performed under sufficient conditions (non-limiting examples of which are described in the Experimental section herein), hepatocytes introduced into a non-human animal engraft and expand within the liver of the non-human in vivo bioreactor. According to some embodiments, the methods comprise monitoring the expansion of the human hepatocytes in the liver of the non-human in vivo bioreactor. Such monitoring may include monitoring for liver function and/or other indicators of health, such as but not limited to body weight, total bilirubin (TBIL), gamma-glutamyl transferase (GGT), glucose, total protein, albumin, aspartate aminotransferase (AST), alkaline phosphatase (ALP), alanine aminotransferase (ALT), and the like. In certain embodiments, animals are assessed and assigned a veterinary clinical score at the time of assessment, including, e.g., where the clinical score included assessments of body condition (e.g., fat, muscle, etc.), observation and scoring of animal behavior, body weight, and hydration status. According to some embodiments, collecting hepatocytes from the liver of the non-human in vivo bioreactor commences based on a clinical score cutoff being met. In some instances, collecting hepatocytes from the liver of the non-human in vivo bioreactor commences based on one or more biomarkers or liver function indicators reaching a threshold value or values where any suitable biomarker or indicator or combination of biomarkers or indicators may be employed. For example, in some instances, collecting hepatocytes may commence when the levels of albumin produced by transplanted hepatocytes (e.g., human albumin produced by transplanted human hepatocytes) reaches a desired threshold. In some instances, collecting hepatocytes may commence when the levels of a combination of two or more, three or more, four or more, five or more, or six or more biomarkers/indicators selected from TBIL, GGT, glucose, total protein, albumin, AST, ALP, ALT, or the like, each reach desired thresholds. In some instances, use of one or more biomarkers/indicators may be combined with a clinical score cutoff to determine when to commence collecting hepatocytes. In some instances, one or more biomarkers/indicators may be used to determine when to commence collection without regard to a clinical score cutoff.

Where used, various clinical scoring matrices may be employed. Useful clinical scoring matrices may include observations of the animals that include assessments of hydration, responsiveness, activity/lethargy, coat/grooming, movement, ear posture, presence or absence of distension, vocalization, eye appearance, skin appearance, bodyweight, swelling, respiration, and the like. Qualitative assessments, such as one or more of the described observational assessments, and/or quantitative assessments may be employed, such as e.g., bodyweight measurements. Each clinical score may be based on a combination of criteria or a single criterion. For example, an alert, hydrated, active, and responsive animal having a normal appearance may be given a clinical score (CS) of 5; an alert, ambulatory, responsive animal having piloerection and an unkempt coat may be given a CS of 4; an animal having the aforementioned characteristics but with retracted ear posture, a hunch, distention, or porphyrin staining may be given a CS of 3.5; a lethargic, quiet, animal with hunched posture and distention (with or without one or more of the preceding characteristics) may be given a CS of 3; a lethargic animal with squinting or sunken eyes, hunched posture, retracted ear posture, unkempt coat, and distention or paraphimosis may be given a CS of 2.5; a lethargic, lean/emaciated animal with the preceding characteristics and distention and paraphimosis may be given a CS of 2; a depressed and moribund animal may be given a clinical score of 1; and an animal found dead may be given a CS of 0. In some instances, clinical scoring using metrics described in Hickman D L and Swan M. (2010) J Am Assoc Lab Anim Sci. 49(2): 155-159 may be employed. In some instances, conversion between metrics may be employed, including but not limited to e.g., where a body condition (BC) score according to Hickman et al. is converted to a CS with or without consideration of other criteria, including e.g., where a BC of 3, 4, or 5 is converted to a CS of 5 or 4; a BC of less than or equal to 3 is converted to a CS of 3.5 or 3; a BC of less than 3 is converted to a CS of 2; a BC of 2 or less is converted to a CS of 1; and the like.

In some instances, a CS may be the lone criterion utilized to select animals for perfusion and/or commence hepatocyte collection. In some instances, a CS may be used in combination with other criteria to select animals for perfusion. In some instances, a CS of a certain value, e.g., CS of 1, CS of 2, CS of 3, CS of 4, or CS of 5, may be used, alone or in combination with other criteria, in selecting animals for perfusion. In some instances, a CS above a certain threshold (i.e., a CS “cutoff”), e.g., a CS of at least 1, a CS of at least 1.5, a CS of at least 2, a CS of at least 2.5, a CS of at least 3, a CS of at least 3.5, a CS of at least 4, or a CS of at least 4.5, may be used, alone or in combination with other criteria, in selecting animals for perfusion. In some instances, animals may be selected for perfusion based on criteria other than a CS, i.e., a CS may not be employed in selecting animals for perfusion and/or commencement of hepatocyte collection.

In certain embodiments, the methods comprise monitoring the expansion of the human hepatocytes in the liver of the non-human in vivo bioreactor, and such monitoring comprises monitoring the level of a circulating biomarker secreted by the human hepatocytes in the non-human in vivo bioreactor during the expanding. A non-limiting example of a circulating biomarker which may be monitored to assess the degree of repopulation of in vivo bioreactor host liver with engrafted human hepatocytes is human albumin (hAlb). A circulating biomarker may be monitored, e.g., in whole blood (e.g., peripheral blood) or a fraction thereof obtained from the non-human in vivo bioreactor. Assays including but not limited to enzyme-linked immunosorbent assay (ELISA) may be readily employed to monitor a circulating biomarker in the non-human in vivo bioreactor. In certain embodiments, collecting hepatocytes from the liver of the non-human in vivo bioreactor commences based on the monitored level of the circulating biomarker reaching a threshold level. For example, when whole blood hAlb levels are used to monitor the expansion of the human hepatocytes in the liver of the non-human in vivo bioreactor, suitable threshold levels include, e.g., 1000 or greater μg/mL, 1500 or greater μg/mL, 2000 or greater μg/mL, 2250 or greater μg/mL, 2500 or greater μg/mL, 2750 or greater μg/mL, 3000 or greater μg/mL, 3250 or greater μg/mL, 3500 or greater μg/mL, 3750 or greater μg/mL, 4000 or greater μg/mL, 4250 or greater μg/mL, 4500 or greater μg/mL, 4750 or greater μg/mL, or 5000 or greater μg/mL, 5500 or greater μg/mL, 6000 or greater μg/mL, 7000 or greater μg/mL, 8000 or greater μg/mL, 9000 or greater μg/mL, or 10,000 or greater μg/mL.

In certain embodiments, the non-human in vivo bioreactor is genetically modified at one or more loci. Genetic modifications may include knock-out or knock-down to generate a non-human in vivo bioreactor that is deficient at one or more loci or activation of one or more target genes. Genetic modifications may be made at multiple loci in any combination (one or more repressive modifications and/or one or more activating modifications). Useful genetic modifications in a non-human in vivo bioreactor may include modifications in various genes including immune genes (e.g., resulting in immunodeficiency), liver function genes (e.g., resulting in liver function deficiency), metabolic genes (e.g., resulting in metabolic deficiency), amino acid catabolism genes (e.g., resulting in deficient amino acid catabolism), and the like.

In certain embodiments, a useful genetically modified non-human in vivo bioreactor is a fumarylacetoacetate hydrolase (fah)-deficient non-human in vivo bioreactor, for example as described in U.S. Pat. Nos. 8,569,573; 9,000,257; 10,470,445 and the like, the disclosures of which are incorporated herein by reference in their entireties. Examples of fah-deficient non-human animals useful as bioreactors and/or useful in the generation of bioreactors are also described in Nicolas et al., Nat Commun (2022) 13(1):5012; Carbonaro et al. Sci Rep (2022) 12(1):14079; Larson et al. Stem Cell Reports (2021) 16(11):2577-2588; Nelson et al. Tissue Eng Part A (2022) 28(3-4):150-160; Gu et al. Mol Ther Methods Clin Dev (2021) 21:530-547; Zhao et al. Front Immunol (2022) 13:950194; Ren et al. Cell Biosci (2022) 12(1):26; Azuma et al. Nat Biotechnol (2007) 25(8):903-10; the disclosures of which are incorporated herein in their entirety. FAH is a metabolic enzyme that catalyzes the last step of tyrosine catabolism. Animals having a homozygous deletion of the Fah gene exhibit altered liver mRNA expression and severe liver dysfunction. Point mutations in the Fah gene have also been shown to cause hepatic failure and postnatal lethality. Humans deficient for Fah develop the liver disease hereditary tyrosinemia type 1 (HT1) and develop liver failure. Fah deficiency leads to accumulation of fumarylacetoacetate, a potent oxidizing agent and this ultimately leads to cell death of hepatocytes deficient for Fah. Thus, Fah-deficient animals can be repopulated with hepatocytes from other species, including humans, containing a functional fah gene. Fah genomic, mRNA and protein sequences for a number of different species are publicly available, such as in the GenBank database (see, for example, Gene ID 29383 (rat Fah); Gene ID 14085 (mouse Fah); Gene ID 610140 (dog FAH); Gene ID 415482 (chicken FAH); Gene ID 100049804 (horse FAH); Gene ID 712716 (rhesus macaque FAH); Gene ID 100408895 (marmoset FAH); Gene ID 100589446 (gibbon FAH); Gene ID 467738 (chimpanzee FAH); and Gene ID 508721 (cow FAH)) and fah genomic loci in other species are readily identifiable through bioinformatics. Fah-deficient animals may include a genetically modified fah locus and may or may not include further genetic modifications at other loci, including for example where such an animal (e.g., rat, pig or mouse) is deficient in FAH, RAG-1 and/or RAG-2, and IL-2Rγ (referred in some instances as an “FRG” animal, such as an FRG mouse, FRG pig, or FRG rat).

Useful genetic modifications also include those resulting in immunodeficiency, e.g., from a lack of a specific molecular or cellular component of the immune system, functionality of a specific molecular or cellular component of the immune system, or the like. In some instances, useful genetic alterations include a genetic alteration of the Recombination activating gene 1 (Rag1) gene. Rag1 is a gene involved in activation of immunoglobulin V(D)J recombination. The RAG1 protein is involved in recognition of the DNA substrate, but stable binding and cleavage activity also requires RAG2. Rag-1-deficient animals have been shown to have no mature B and T lymphocytes. In some instances, useful genetic alterations include a genetic alteration of the Recombination activating gene 2 (Rag2) gene. Rag2 is a gene involved in recombination of immunoglobulin and T cell receptor loci. Animals deficient in the Rag2 gene are unable to undergo V(D)J recombination, resulting in a complete loss of functional T cells and B cells (see e.g., Shinkai et al. Cell 68:855-867, 1992). In some instances, useful genetic alterations include a genetic alteration of the common-gamma chain of the interleukin receptor (Il2rg). Il2rg is a gene encoding the common gamma chain of interleukin receptors. Il2rg is a component of the receptors for a number of interleukins, including IL-2, IL-4, IL-7 and IL-15 (see e.g., Di Santo et al. Proc. Natl. Acad. Sci. U.S.A. 92:377-381, 1995). Animals deficient in Il2rg exhibit a reduction in B cells and T cells and lack natural killer cells. Il2rg may also be referred to as interleukin-2 receptor gamma chain.

Examples of animal models useful in hepatocyte transplantation (and the relevant genes involved) either alone or crossed/combined with one or more other mutations or transgenes, include e.g., Fah−/−mouse, (Fumarylacetoacetate hydrolase), Mdr2−/− mouse (Multidrug resistance protein 2), uPA+/+ mouse (Urokinase-type plasminogen activator), Rag2−/−gamma(c)−/− mouse (Interleukin 2 receptor gamma chain), DPPIV rat (Dipeptidyl peptidase IV), Gunn rat (Uridine diphosphoglucuronate glucuronosyltransferase-1A1), Long-Evans Cinnamon rat (ATPB7), Watanabe rabbit (LDL receptor), and the like, e.g., as described in Weber et al. Liver Transplantation (2009) 15(1):7-14; the disclosure of which is incorporated herein by reference in its entirety. Others include FRGN mouse (Yecuris), cDNA-uPA/SCID mice (PhoenixBio), TK-NOG mice (Hera Biolabs), SRG rat/HepatoRat (Hera Biolabs), and the like.

In some instances, non-human in vivo bioreactors may be immunosuppressed, including e.g., where immunosuppression is achieved through administration of one or more immunosuppressive agents. Any suitable immunosuppressive agent or agents effective for achieving immunosuppression in the non-human in vivo bioreactor can be used. Examples of immunosuppressive agents include, but are not limited to, FK506, cyclosporin A, fludarabine, mycophenolate, prednisone, rapamycin and azathioprine. Combinations of immunosuppressive agents can also be administered. In some instances, immunosuppressive agents are employed in place of genetic immunodeficiency. In some instances, immunosuppressive agents are employed in combination with genetic immunodeficiency.

As summarized herein, genetically modified non-human in vivo bioreactors may include one or more (i.e., a combination of) genetic modifications. For example, such a non-human in vivo bioreactor may include a rag1 genetic modification, a rag2 genetic modification, a IL2rg genetic modification, or such an animal may include a rag1 or rag2 genetic modification and a genetic alteration of the Il2rg gene such that the genetic alteration correspondingly results in loss of expression of functional RAG1 protein, RAG2 protein, IL-2rg protein, or RAG-1/RAG-2 protein and IL-2rg protein. In one example, the one or more genetic alterations include a genetic alteration of the Rag2 gene and a genetic alteration of the Il2rg gene. In one example, the one or more genetic alterations include a genetic alteration of the Rag1 gene and a genetic alteration of the Il2rg gene. In one example, the one or more genetic alterations include a genetic alteration of the Rag1 gene, a genetic alteration of the Rag2 gene, and a genetic alteration of the Il2rg gene. In some instances, useful genetic alterations include e.g., SCID, NOD, SIRPα, perforin, or nude. Altered loci may be genetic nulls (i.e., knockouts) or other modifications resulting in deficiencies in the gene product at the corresponding loci. Specific cells of the immune system (such as macrophages or NK cells) can also be depleted. Any convenient method of depleting particular cell types may be employed.

It will be appreciated that various models of liver injury, creating a selective growth advantage for hepatocyte xenografts, may be used in a non-human in vivo bioreactor (e.g., rat, pig, mouse, rabbit) to facilitate hepatocyte engraftment and expansion, including, without limitation, inducible injury, selective embolism, transient ischemia, retrorsine, monocrotoline, thioacetamide, irradiation with gamma rays, carbon tetrachloride, and/or genetic modifications (e.g., Fah disruption, uPA, TK-NOG (Washburn et al., Gastroenterology, 140(4):1334-44, 2011), albumin AFC8, albumin diphtheria toxin, Wilson's Disease, any of genetic modifications present in the liver-deficient animal models described herein, and the like). Combinations of liver injury techniques may also be used.

In some embodiments, the non-human in vivo bioreactor is administered a vector (e.g., an adenovirus (Ad) vector) encoding a urokinase gene (e.g., urokinase plasminogen activator (uPA)) prior to injection of the heterologous hepatocytes. Expression of uPA in hepatocytes causes hepatic injury and thus permits the selective expansion of hepatocyte xenografts upon transplantation. In one embodiment, the urokinase gene is human urokinase and may be secreted or non-secreted. See, e.g., U.S. Pat. Nos. 8,569,573; 9,000,257; 10,470,445 and the like. In some instances, a vector may be administered one or more (or a fraction of one day) prior to hepatocyte transplantation, e.g., to precondition the recipient for hepatocyte engraftment, including but not limited to e.g., 6 hours to 5 days, 6 hours to 3 days, 12 hours to 3 days, 12 hours to 2 days, 6 hours to 36 hours, 12 hour to 36 hours, 6 hours to 24 hours, or 12 hours to 24 hours prior to hepatocyte transplantation.

In some instances, a TK-NOG liver injury model (i.e., an albumin thymidine kinase transgenic-NOD-SCID-interleukin common gamma chain knockout) may be used as the non-human in vivo bioreactor as described herein. TK-NOG animals include a herpes simplex virus thymidine kinase hepatotoxictransgene that can be conditionally activated by administration of ganciclovir. Hepatic injury resulting from activation of the transgene during administration of ganciclovir provides a selective advantage to hepatocyte xenografts, facilitating use of such animals as in vivo bioreactors for the expansion of transplanted hepatocytes as described herein.

In some instances, an AFC8 liver injury model (characterized as having a FKBP-Caspase 8 gene driven by the albumin promoter) may be used as the non-human in vivo bioreactor as described herein. AFC8 animals include a FK508-caspase 8 fusion hepatotoxic transgene that can be conditionally activated by administration of AP20187. Hepatic injury resulting from activation of the transgene during administration of AP20187 provides a selective advantage to hepatocyte xenografts, facilitating use of such animals as in vivo bioreactors for the expansion of transplanted hepatocytes.

In some instances, an NSG-PiZ liver injury model (characterized as having an α-1 antitrypsin (AAT) deficiency combined with immunodeficiency (NGS)) may be used as a non-human in vivo bioreactor. NSG-PiZ animals have impaired secretion of AAT leading to the accumulation of misfolded PiZ mutant AAT protein triggering hepatocyte injury. Such hepatic injury provides a selective advantage to hepatocyte xenografts, facilitating use of such animals as in vivo bioreactors for the expansion of transplanted hepatocytes. The immunodeficiency renders the animal capable of hosting a xenograft without significant rejection.

In some instances, an animal may be preconditioned to improve the recipient liver's ability to support the transplanted cells. Various preconditioning regimens may be employed, including but not limited to e.g., irradiation preconditioning (e.g., partial liver irradiation), embolization preconditioning, ischemic preconditioning, chemical/viral preconditioning (using e.g., uPA, cyclophosphamide, doxorubicin, nitric oxide, retrorsine, monocrotaline, toxic bile salts, carbon tetrachloride, thioacetamide, and the like), liver resection preconditioning, and the like. In some instances, hepatocyte-generating cells may be introduced in the absence of preconditioning and/or a procedure will specifically exclude one, all, or some combination of preconditioning regimens or specific reagents, including e.g., one or more of those described herein. In some instances, induction of liver injury through cessation of NTBC or administration of ganciclovir or AP20187 may be used for preconditioning. Where employed, preconditioning may be performed at some time, including hours, days, or weeks or more, prior to transplantation of hepatocyte-generating cells, including e.g., at least 6 hours, at least 12 hours, at least 24 hours, at least 36 hours, at least 48 hours, at least 60 hours, at least 72 hours, at least 4 days, at least 5 days, at least 6 days, at least a week, or at least two weeks at least prior to transplantation.

After optional pre-conditioning (e.g., with uPA) of the non-human in vivo bioreactor (e.g., 24 hours after pre-conditioning), heterologous hepatocytes can be delivered to the non-human in vivo bioreactor via any suitable method. In certain embodiments, the hepatocytes as described herein are administered directly to the liver (e.g., via portal vein injection) and/or via intra-splenic injection where the hepatocytes will travel through the vasculature to reach the liver. In certain embodiments, anywhere between 1×10⁵ and 1×10⁹ (e.g., 5×10⁵/mouse, 5-10×10⁶/rat, etc.) hepatocytes are introduced into an animal (e.g., an FRG animal), optionally preconditioned (e.g., 24 hours prior to administration), e.g., with adenoviral uPA (e.g., 1.25×10⁹ PFU/25 grams of mouse body weight). The number of hepatocytes introduced into the non-human in vivo bioreactor will vary and may range, e.g., depending on various factors including the species and size of the animal receiving the cells, from 1×10⁵ or less to 1×10⁹ or more, including but not limited to e.g., 1×10⁵ to 1×10⁹, 1×10⁶ to 1×10⁹, 1×10⁷ to 1×10⁹, 1×10⁸ to 1×10⁹, 1×10⁵ to 1×10⁶, 1×10⁵ to 1×10⁷, 1×10⁵ to 1×10⁸, 1×10⁶ to 1×10⁷, 1×10⁷ to 1×10⁸, 1×10⁶ to 1×10⁸, etc. In some instances, the number of cells administered may be 1×10⁹ or less, including e.g., 0.5×10⁹ or less, 1×10⁸ or less, 0.5×10⁸ or less, 1×10⁷ or less, 0.5×10⁷ or less, 1×10⁶ or less, 0.5×10⁶ or less, 1×10⁵ or less, etc. Hepatocytes introduced into a bioreactor (or non-human animal generally) may vary and such cells may be allogenic or heterologous with respect to the non-human in vivo bioreactor (or non-human animal generally).

In addition, immune suppression drugs can optionally be given to the animals before, during and/or after the transplant to eliminate the host versus graft response in the non-human in vivo bioreactor (e.g., the rat, pig or mouse) from xenografted heterologous hepatocytes. In some instances, by cycling the animals off immune suppression agents for defined periods of time, the liver cells become quiescent and the engrafted cells will have a proliferative advantage leading to replacement of endogenous hepatocytes (e.g., mouse, pig, or rat hepatocytes) with heterologous hepatocytes (e.g., human hepatocytes). In the case of human hepatocytes, this generates animals with high levels of humanization of the liver, i.e., humanized livers. Heterologous hepatocyte repopulation levels can be determined through various measures, including but not limited to e.g., quantitation of human serum albumin levels, optionally correlated with immunohistochemistry of liver sections from transplanted animals.

In some embodiments, an agent that inhibits, delays, avoids or prevents the development of liver disease is administered to the non-human in vivo bioreactor during the period of expansion of the administered hepatocytes. Administration of such an agent avoids (or prevents) liver dysfunction and/or death of the non-human in vivo bioreactor (e.g., rat, pig or mouse bioreactor) prior to repopulation of the non-human in vivo bioreactor (e.g., rat, pig or mouse bioreactor) with healthy (e.g., FAH-expressing) heterologous hepatocytes. The agent can be any compound or composition that inhibits liver disease in the disease model relevant to the bioreactor. One such agent is 2-(2-nitro-4-trifluoro-methyl-benzoyl)-1,3 cyclohexanedione (NTBC), but other pharmacologic inhibitors of phenylpyruvate dioxygenase, such as methyl-NTBC can be used. NTBC is administered to regulate the development of liver disease in a Fah-deficient animal. The dose, dosing schedule and method of administration can be adjusted, and/or cycled, as needed to avoid catastrophic liver dysfunction, while promoting expansion of hepatocyte xenografts, in the Fah-deficient non-human in vivo bioreactor. In some embodiments, the Fah-deficient animal is administered NTBC for at least two days, at least three days, at least four days, at least five days or at least six days following transplantation of hepatocytes as described herein. In some embodiments, the Fah-deficient animal is further administered NTBC for at least about one week, at least about two weeks, at least about three weeks, at least about four weeks, at least about one month, at least about two months, at least about three months, at least about four months, at least about five months, or at least about six months. In some embodiments, the NTBC (or another compound with a liver protective effect) is withdrawn at about two days, about three days, about four days, about five days, about six days or about seven days following hepatocyte transplantation.

The dose of NTBC administered to the Fah-deficient animal can vary. In some embodiments, the dose is about 0.5 mg/kg to about 30 mg/kg per day, e.g., from about 1 mg/kg to about 25 mg/kg, from about 10 mg/kg per day to about 20 mg/kg per day, or about 20 mg/kg per day. NTBC can be administered by any suitable means, such as, but limited to, in the drinking water, in the food or by injection. In one embodiment, the concentration of NTBC administered in the drinking water is about 1 to about 30 mg/L, e.g., from about 10 to about 25 mg/L, from about 15 to about 20 mg/L, or about 20 mg/L. In certain embodiments, NTBC administration is cyclical from before transplantation to 4 to 8 or more weeks post-transplantation. In certain embodiments, NTBC administration is cyclical for the entire, or essentially the entire, transplanted hepatocyte expansion period, i.e., the period following transplantation of the hepatocytes into the in vivo bioreactor until hepatocyte expansion reaches a desired level of expansion prior to collection from the in vivo bioreactor.

In some instances, a hepatocyte population, e.g., a hepatocyte population derived from a single donor, a hepatocyte population derived from a specific pool of donors, a hepatocyte population from a single master cell bank, etc., may be expanded in multiple (i.e., a plurality) of individual in vivo bioreactors, including e.g., where the hepatocytes of the population are expanded in a plurality of bioreactors in parallel and/or in series. Where a plurality of individual bioreactors are employed, in some instances, expansion may be monitored in each individual (or some subset of the plurality) and a determination to harvest the expanded hepatocytes may be made individually for each animal based on the monitoring, collectively for the plurality (e.g., based on sampling one or more, or all of the, animals of the plurality), or one or more subgroups of the plurality (e.g., based on sampling one or more, or all of the, animals of the plurality or the subgroup(s)).

According to the hepatocyte production methods of the present disclosure, hepatocytes collected from the liver of the non-human in vivo bioreactor comprise a xenomixture of expanded human hepatocytes and non-human in vivo bioreactor cells, including non-human in vivo bioreactor hepatocytes, and the xenomixture is subjected to hepatocyte collection procedures. Useful collection procedures include centrifugal elutriation (a technique for separating particles (e.g., cells) based on size and density using an elutriation rotor), which is performed under conditions sufficient to produce an elutriation fraction enriched for the expanded human hepatocytes. For elutriation, in some embodiments, a container comprising the xenomixture may be connected to multiple containers, including e.g., a container of elutriation buffer (EB), a hepatocyte collection container, and a waste collection container. The connected containers and associated tubing may then be connected to an elutriator, e.g., a Gibco™ CTS™ Rotea™ Counterflow Centrifugation System, a standard or custom Counter-Flow Centrifugation system, or other suitable elutriator. Using the elutriator, tubing lines may be primed with EB followed by formation of a cell bed within the elutriation chamber using the cell suspension comprising the xenomixture. As noted elsewhere herein and demonstrated in the Experimental section below, the hepatocyte production methods of the present disclosure are based in part on the unexpected finding that human hepatocytes may be separated, at least partially, from non-human in vivo bioreactor hepatocytes via elutriation. As also noted elsewhere herein and demonstrated in the Experimental section below, the hepatocyte production methods of the present disclosure are based in part on the unexpected finding that human hepatocytes processed using an elutriation-based procedure are superior in function to hepatocytes from the same donor liver processed using a primarily density sedimentation-based, density-centrifugation-based (e.g., Percoll-based) procedure. By varying the centrifugal force in the elutriation chamber under a constant flow rate, an elutriation fraction that preferentially contains human hepatocytes may be retained, washed and then collected from the elutriator into a sterile collection container. In some embodiments, the elutriation is performed at a constant or varying centrifugal force of from 100×g to 4000×g, 100×g to 3000×g, 100×g to 2500×g, 100×g to 2000×g, 100×g to 1500×g, 100×g to 1000×g, 200×g to 4000×g, 300×g to 4000×g, 400×g to 4000×g, 500×g to 4000×g, 600×g to 4000×g, 700×g to 4000×g, 800×g to 4000×g, 200×g to 2000×g, 300×g to 2000×g, 400×g to 2000×g, 500×g to 2000×g, 600×g to 2000×g, 700×g to 2000×g, 800×g to 2000×g, 200×g to 1000×g, 300×g to 1000×g, 400×g to 1000×g, 500×g to 1000×g, 2000×g to 4000×g (e.g., 2250 to 3750×g, 2500 to 3500×g, 2750 to 3250×g (e.g., about 3000×g)) and a peristaltic pump flow rate of from 5 to 160 mL/min, including e.g., from from 10 to 150 mL/min, from 20 to 140 mL/min, from 30 to 130 mL/min, from 40 to 120 mL/min, from 50 to 110 mL/min, from 50 to 150 mL/min, from 60 to 140 mL/min, from 70 to 130 mL/min, from 80 to 120 mL/min, from 90 to 110 mL/min, from 5 to 25 mL/min, from 25 to 50 mL/min, from 50 to 75 mL/min, from 75 to 100 mL/min, from 100 to 125 mL/min, from 125 to 150 mL/min, etc. In certain embodiments, the expanded human hepatocytes constitute 50% or greater, 60% or greater, or 70% or greater of the total cells present in the elutriation fraction enriched for the expanded human hepatocytes.

In some instances, where desired a lower g-force may be utilized and compensated by a corresponding decrease in flow rate. In some instances, where a higher flow rate is desired a compensatory increase in g-force may be utilized. In some instances, settings may be calibrated to compensate for use of an alternative elutriator.

Embodiments of the hepatocyte production methods of the present disclosure comprise removing non-human in vivo bioreactor cells from a xenomixture, such as e.g., an elutriation fraction, via a negative selection process. “Negative selection” as used herein is a process by which non-human in vivo bioreactor cells are removed from the elutriation fraction, or other xenomixture, via targeting and sequestering the non-human in vivo bioreactor cells from the expanded human hepatocytes present within a xenomixture, such as e.g., an elutriation fraction. In some instances, negative selection may be performed following elutriation. In some instances, negative selection may be performed prior to elutriation. In some instances, multiple instances of elutriation and/or negative selection may be performed. In some instances, a single instance of elutriation may be performed in a hepatocyte isolation process, e.g., before and/or after one or more instances of negative selection. In some instances, a single instance of negative selection may be performed in a hepatocyte isolation process, e.g., before and/or after one or more instances of elutriation.

In certain embodiments, the negative selection process is an antibody-based negative selection process. For example, the negative selection process may comprise contacting a xenomixture, such as e.g., an elutriation fraction, with a primary antibody specific for non-human in vivo bioreactor cells under conditions sufficient for specific binding of the primary antibody to non-human in vivo bioreactor cells present in the xenomixture, and removing non-human in vivo bioreactor cells from the xenomixture utilizing the primary antibody. In certain embodiments, removing non-human in vivo bioreactor cells utilizing the primary antibody comprises contacting the primary antibody with a labeled secondary antibody under conditions sufficient for binding of the secondary antibody to the primary antibody, and utilizing the label of the labeled secondary antibody to remove, from the xenomixture, complexes comprising labeled secondary antibody, primary antibody, and a non-human in vivo bioreactor cell. According to some embodiments, the primary antibody is labeled, and removing non-human in vivo bioreactor cells comprises utilizing the label to remove, from the xenomixture, complexes comprising primary antibody and a non-human in vivo bioreactor cell.

When the negative selection process employs a labeled secondary or primary antibody, any suitable label may be employed. In certain embodiments, the label comprises an affinity tag. Non-limiting examples of affinity tags include biotin, avidin, streptavidin, an aptamer, an MS2 coat protein-interacting sequence, a U1A protein-interacting sequence, etc. According to some embodiments, the label is magnetically responsive, thereby permitting magnetic-based negative selection of antibody-bound non-human in vivo bioreactor cells. For example, a labeled secondary or primary antibody employed in a magnetic-based negative selection process may be labeled with a magnetic bead, e.g., a magnetic bead-bound secondary antibody or a magnetic bead-bound primary antibody. According to such embodiments, negative selection may comprise applying a magnetic force to a container/vessel (e.g., a flow-through column, a collection vessel (e.g., a collection bag)) comprising the elutriation fraction which has been contacted under antibody binding conditions with the magnetically labeled secondary or primary antibody, thereby sequestering non-human in vivo bioreactor cells from the expanded human hepatocytes. Non-limiting example approaches that may be employed for magnetic-based negative selection of antibody-bound non-human in vivo bioreactor cells are described in detail in the Experimental section herein.

The term “antibody” (also used interchangeably with “immunoglobulin”) encompasses polyclonal (e.g., rabbit polyclonal) and monoclonal antibody preparations where the antibody may be an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the compound, including, but not limited to single chain Fv (scFv), Fab, (Fab′)₂, (scFv′)₂, and diabodies; chimeric antibodies; monoclonal antibodies, humanized antibodies, human antibodies; and fusion proteins comprising an antigen-binding portion of an antibody and a non-antibody protein. In some embodiments, the antibody is selected from an IgG, Fv, single chain antibody, scFv, a Fab, a F(ab′)₂, and a F(ab′). The antibodies may be further conjugated to other moieties, such as members of specific binding pairs, e.g., biotin (member of biotin-avidin specific binding pair), affinity tags, and/or the like.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, MD (1987); and Lefranc et al. IMGT, the international ImMunoGeneTics information System®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, an antibody of the present disclosure is an IgG antibody, e.g., an IgG1 antibody, such as a human IgG1 antibody.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

According to some embodiments, an antibody of the present disclosure is a monoclonal antibody. “Monoclonal antibody” refers to a composition comprising one or more antibodies obtained from a population of substantially homogeneous antibodies, i.e., a population the individual antibodies of which are identical except for any naturally occurring mutations that may be present in minor amounts. Monoclonal antibodies are highly specific, being directed against a single antigenic site and generally to a single epitope on an antigen. The modifier “monoclonal” indicates the character of the antibody as being obtained from a substantially homogeneous population of antibodies, and does not require that the antibody be produced by any particular method or be the only antibody in the composition.

In certain embodiments, when an antibody-based negative selection process is employed, the primary antibody is a pan-non-human in vivo bioreactor antibody. The term “pan-non-human in vivo bioreactor antibody”, as used herein, refers to an antibody that, under antibody binding conditions, binds to an antigen expressed on the surface of all or substantially all non-human in vivo bioreactor cells, which antigen is not expressed on the surface of human cells, including human hepatocytes expanded according to the methods of the present disclosure. According to some embodiments, the pan-non-human in vivo bioreactor antibody is an anti-histocompatibility antigen antibody, i.e., specifically binds to a non-human in vivo bioreactor histocompatibility antigen. In some embodiments, when the non-human in vivo bioreactor is a rat, the pan-non-human in vivo bioreactor antibody specifically binds a rat cell surface antigen selected from rat RT1A class I histocompatibility antigen (“RT1A”), rat dipeptidyl peptidase 4 (“CD26”), rat membrane cofactor protein (“CD46”), rat transferrin receptor protein 1 (“CD71”), and rat H-2 class II histocompatibility antigen gamma chain (“CD74”), details of which are provided in the Experimental section herein.

Accordingly, in some embodiments, when the non-human in vivo bioreactor is a rat and an antibody-based negative selection process is implemented, the methods may employ an anti-RT1A primary antibody. Non-limiting examples of such antibodies include those that compete for binding to RT1A with the monoclonal IgG1 MRC clone OX-18 (see e.g., Fukumoto, T. et al. (1982) Eur J Immunol. 12 (3): 237-43; herein “OX-18”), monoclonal IgG2a MRC clone OX-27 (see e.g., Jefferies et al. (1985) J Exp Med. 162(1):117-27; herein “OX-27”), and/or monoclonal IgG1 clone F16-4-4 (see e.g., Hart & Fabre (1981) Transplantation. 31(5):318-325; herein “F-16”). Whether an antibody of the present disclosure “competes with” a second antibody for binding to the antigen may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is added. One of the two antibodies is labeled. If the labeled antibody and the unlabeled antibody bind to separate and discrete sites on the antigen, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the antigen by about 50% or more, about 60% or more, about 70% or more, about 80% or more, about 85% or more, about 90% or more, about 95% or more, or about 99% or more. Details of procedures for carrying out such competition assays are known and can be found, for example, in Harlow and Lane, Antibodies, A Laboratory Manual, Cold Spring Harbor Laboratory Press, Cold Spring Harbor, New York, 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

In some embodiments, the anti-RT1A primary antibody employed specifically binds RT1A and comprises—or competes for binding to RT1A with an antibody comprising—one, two, three, four, five, or all six CDRs of an antibody designated herein as OX-18, OX-27 or F-16. For example, the human hepatocyte production methods of the present disclosure may employ a primary antibody that specifically binds RT1A and comprises—or competes for binding to RT1A with an antibody comprising—one, two, three, four, five, or all six CDRs of an antibody designated herein as OX-18, OX-27 or F-16. In some embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of an antibody designated herein as antibody OX-18, OX-27 or F-16; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of an antibody designated herein as antibody OX-18, OX-27 or F-16; or both.

As such, according to some embodiments, the anti-RT1A antibody comprises, or competes for binding to RT1A with an antibody comprising:
a variable heavy chain (V_{H}) polypeptide comprising:
   a V_{H} CDR1 comprising the amino acid sequence GDSITSGY (SEQ ID NO:1),
   a V_{H} CDR2 comprising the amino acid sequence ISYSGST (SEQ ID NO:2), and
   a V_{H} CDR3 comprising the amino acid sequence ASHSHWYFDV (SEQ ID NO:3), and
a variable light chain (V_{L}) polypeptide comprising:
   a V_{L} CDR1 comprising the amino acid sequence QDISNY (SEQ ID NO:4),
   a V_{L} CDR2 comprising the amino acid sequence YTS (SEQ ID NO:5), and
   a V_{L} CDR3 comprising the amino acid sequence QQGNTLPWT (SEQ ID NO:6),
wherein CDRs are defined according to IMGT.

The phrases “specifically binds”, “specific for”, “immunoreactive” and “immunoreactivity”, and “antigen binding specificity”, when referring to an antibody, refer to a binding reaction with an antigen which is highly preferential to the antigen or a fragment thereof, so as to be determinative of the presence of the antigen in the presence of a heterogeneous population of antigens (e.g., proteins and other biologics, e.g., in a sample). Thus, under designated immunoassay conditions, the specified antibodies bind to a particular non-human in vivo bioreactor antigen and do not bind in a significant amount to other antigens present in the sample. Specific binding to an antigen under such conditions may require an antibody that is selected for its specificity for a particular antigen. For example, an anti-non-human in vivo bioreactor antigen antibody can specifically bind to a non-human in vivo bioreactor antigen, and does not exhibit comparable binding (e.g., does not exhibit detectable binding) to other antigens (e.g., proteins) present in a sample, such as e.g., human antigens.

In some embodiments, an antibody of the present disclosure “specifically binds” a non-human in vivo bioreactor antigen if it binds to or associates with the non-human in vivo bioreactor antigen (e.g., RT1A) with an affinity or Kₐ (that is, an equilibrium association constant of a particular binding interaction with units of 1/M) of, for example, greater than or equal to about 10⁵ M⁻¹. In certain embodiments, the antibody binds to the non-human in vivo bioreactor antigen with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the antibody binds to the non-human in vivo bioreactor antigen with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for the non-human in vivo bioreactor antigen can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

The present disclosure provides antibodies that specifically bind non-human in vivo bioreactor antigens, such as e.g., the non-human in vivo bioreactor antigen RT1A. Such antibodies may include a V_{H} polypeptide and a V_{L} polypeptide that each include combinations of CDRs, such as e.g., any such combinations described in Table 2. In some instances, useful antibody may be derived from the variable regions of one or both of the V_{H} and V_{L} polypeptides of an antibody described herein. Such derived antibodies may be multi-chain or single-chain antibody. For example, in some instances, a useful antibody derived from V_{H} and/or V_{L} polypeptides of an antibody described herein may be a scFv, a Fab, a (Fab′)2, a (scFv′)2, a diabody, a nanobody, or the like.

Also provided are nucleic acids encoding one polypeptide (i.e., a V_{H} polypeptide or a V_{L} polypeptide), both polypeptides (e.g., a V_{H} polypeptide and a V_{L} polypeptide), one or more portions of a V_{H} polypeptide, or one or more portions of a V_{L} polypeptide of an antibody described herein. Such nucleic acids may include e.g., a coding region encoding a V_{H} polypeptide or a V_{L} polypeptide. In some cases, a useful nucleic acid will include two coding regions, one region encoding a V_{H} polypeptide and one region encoding a V_{L} polypeptide, including where such regions may be separated by a regulatory element, such as an IRES, or a sequence encoding a self-cleaving peptide, such as e.g., a T2A or P2A sequence, or the like, allowing for expression of both coding sequences from one nucleic acid. In some instances, a V_{H} polypeptide and a V_{L} polypeptide may be on separate nucleic acids. In some instances, encoding nucleic acids may be modified for expression in mammalian cells, including e.g., where the sequence is mammalian codon optimized.

Such nucleic acids may be present in a vector. A “vector” or “expression vector” is a replicon, such as plasmid, phage, virus, or cosmid, to which another DNA segment, i.e. an “insert”, may be attached so as to bring about the replication of the attached segment in a cell. Coding sequences may be operably linked to one or more regulatory elements, such as e.g., a promoter, enhancer, etc. “Operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may, but need not, necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

In certain embodiments, the human hepatocyte production methods of the present disclosure do not comprise a step of centrifugal sedimentation to enrich for expanded human hepatocytes. According to some embodiments, the isolated expanded human hepatocytes produced according to the methods of the present disclosure exhibit improved cell fitness as compared to a comparable human hepatocyte population isolated using centrifugal sedimentation to separate the human hepatocytes from in vivo bioreactor cells. In certain embodiments, the isolated expanded human hepatocytes produced according to the methods of the present disclosure exhibit equivalent or improved cell fitness as compared to the human hepatocytes introduced into the liver of the non-human in vivo bioreactor. According to some embodiments, the isolated expanded human hepatocytes produced according to the methods of the present disclosure exhibit equivalent or improved cell fitness as compared to a comparable previously cryopreserved, freshly thawed human cadaveric hepatocyte population.

Improved cell fitness of the isolated expanded human hepatocytes produced according to the methods of the present disclosure may be measured by an assay for attachment efficiency, ammonia detoxification, human albumin expression, A1AT expression, CYP3A4, or any combination thereof. In certain embodiments, the improved cell fitness is measured by an in vivo function assay, such as e.g., an in vivo human albumin assay. The in vivo human albumin assay may be an ELISA, including but not limited to an hAlb ELISA, e.g., as measured in a whole blood or serum sample, as described in the Experimental section herein.

According to some embodiments, the human hepatocyte production methods of the present disclosure comprise: introducing human hepatocytes into the livers of a plurality of non-human in vivo bioreactors; expanding the human hepatocytes in the livers of the non-human in vivo bioreactors; collecting hepatocytes from the livers of the non-human in vivo bioreactors, wherein the collected hepatocytes comprise a xenomixture of expanded human hepatocytes and non-human in vivo bioreactor hepatocytes; subjecting the xenomixture to centrifugal elutriation under conditions sufficient to produce an elutriation fraction enriched for the expanded human hepatocytes; and removing non-human in vivo bioreactor cells from the elutriation fraction via a negative selection process to produce isolated expanded human hepatocytes. In certain embodiments, such methods comprise pooling the hepatocytes collected from the livers of the non-human in vivo bioreactors during the collecting, after the collecting, before the elutriation, during the elutriation, after the elutriation, before the negative selection process, during the negative selection process, or after the negative selection process. According to any of the human hepatocyte production methods of the present disclosure, the human hepatocytes are derived from a single human donor. According to any of the human hepatocyte production methods of the present disclosure, the human hepatocytes may be derived from two or more human donors.

As will be appreciated with the benefit of the present disclosure, also provided herein are centrifugal elutriation-based and/or negative selection-based methods of enriching for human hepatocytes in a xenomixture. For example, aspects of the present disclosure include methods of enriching for human hepatocytes in a xenomixture, the methods comprising subjecting a xenomixture comprising human hepatocytes and at least one type of non-human hepatocytes to centrifugal elutriation under conditions sufficient to produce an elutriation fraction enriched for the human hepatocytes. In some embodiments, the non-human hepatocytes are deficient for fumarylacetoacetate hydrolase (Fah). In certain embodiments, the xenomixture comprises rodent hepatocytes, e.g., rat hepatocytes. In some embodiments, the non-human hepatocytes are rodent hepatocytes deficient for interleukin 2 receptor subunit gamma (IL2rg), a recombination activating gene 1 (RAG1), a recombination activating gene 2 (RAG2), or a combination thereof.

Also by way of example, provided are methods of enriching for human hepatocytes in a xenomixture, the methods comprising subjecting a xenomixture comprising human hepatocytes and non-human hepatocytes to an antibody-based negative selection process. In some embodiments, the xenomixture is produced from the liver of an in vivo bioreactor comprising the human hepatocytes and non-human hepatocytes. According to some embodiments, the antibody-based negative selection process comprises contacting the xenomixture with a primary antibody specific for the non-human hepatocytes under conditions sufficient for specific binding of the primary antibody to the non-human hepatocytes, and removing the non-human hepatocytes from the xenomixture utilizing the primary antibody. In some embodiments, removing the non-human hepatocytes from the xenomixture utilizing the primary antibody comprises contacting the antibody with a labeled secondary antibody under conditions sufficient for binding of the secondary antibody to the primary antibody, and utilizing the label of the labeled secondary antibody to remove from the xenomixture complexes comprising the labeled secondary antibody, the primary antibody, and the non-human hepatocyte. In some embodiments, the primary antibody is labeled, and removing the non-human hepatocytes from the xenomixture comprises utilizing the label to remove from the xenomixture complexes comprising the primary antibody and the non-human hepatocyte. Primary and optional secondary antibodies that may be employed in the methods of enriching for human hepatocytes in a xenomixture include those described elsewhere herein in the context of the methods of producing isolated expanded human hepatocytes.

### Isolated Expanded Human Hepatocytes and Compositions

Aspects of the present disclosure further include isolated expanded human hepatocytes and related compositions.

In certain embodiments, provided are isolated expanded human hepatocytes produced according to any of the methods of the present disclosure for producing isolated expanded human hepatocytes. According to some embodiments, such isolated expanded human hepatocytes are derived from a single human donor. In certain embodiments, the isolated expanded human hepatocytes are cryopreserved. As used herein, “cryopreserved” refers to a cell (such as a hepatocyte) or tissue that has been preserved or maintained by cooling to low sub-zero temperatures, such as 77 K or −196 deg. C. (the boiling point of liquid nitrogen). At these low temperatures, any biological activity, including the biochemical reactions that would lead to cell death, is effectively stopped. Useful methods of cryopreservation and thawing cryopreserved cells, as well as processes and reagents related thereto, include but are not limited to e.g., those described in U.S. Pat. Nos. 10370638; 10159244; 9078430; 7604929; 6136525; and 5795711, the disclosures of which are incorporated herein by reference in their entirety. In contrast, the term “fresh”, as used herein with reference to cells, may refer to cells that have not been cryopreserved and, e.g., may have been directly obtained and/or used (e.g., transplanted, cultured, etc.) following collection from a subject or organ thereof.

According to some embodiments, provided is a population of at least 1 billion, including but not limited to e.g., at least 2 billion, at least 3 billion, at least 4 billion, at least 5 billion, at least 6 billion, at least 7 billion, at least 8 billion, at least 9 billion, at least 10 billion, etc., of the isolated expanded human hepatocytes of the present disclosure, optionally wherein the population is present in a single container.

Also provided are compositions comprising the isolated expanded human hepatocytes of the present disclosure. In certain embodiments, a composition of the present disclosure includes the isolated expanded human hepatocytes present in a liquid medium. The liquid medium may be an aqueous liquid medium, such as water, a buffered solution, a cryopreservation solution, or the like. In some instances, the liquid medium may include one or more components of a pharmaceutical preparation.

Also provided are compositions comprising a cell population derived from a xenomixture, the xenomixture comprising dissociated human hepatocytes and at least one type of non-human hepatocytes, the cell population comprising at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% human hepatocytes, optionally wherein the composition comprises 70% or less, including e.g., 65% or less, 60% or less, 55% or less, 50% or less, 45% or less, 40% or less, 35% or less, 30% or less, 25% or less, 20% or less, 15% or less, 10% or less, 9% or less, 8% or less, 7% or less, 6% or less, 5% or less, 4% or less, 3% or less, 2% or less, or 1% or less, non-human hepatocytes.

Aspects of the present disclosure further include pharmaceutical preparations suitable for delivery to a human subject (e.g., directly or indirectly to the liver of a human subject), the pharmaceutical preparation comprising a composition of the present disclosure, optionally wherein the composition comprises at least 1 billion, including but not limited to e.g., at least 2 billion, at least 3 billion, at least 4 billion, at least 5 billion, at least 6 billion, at least 7 billion, at least 8 billion, at least 9 billion, at least 10 billion, etc., of the human hepatocytes. In some embodiments, the at least 1 billion, at least 2 billion, at least 3 billion, at least 4 billion, at least 5 billion, at least 6 billion, at least 7 billion, at least 8 billion, at least 9 billion, at least 10 billion, etc., hepatocytes are derived from a single human donor.

In some instances, compositions present in a single container (e.g., a cryovial or a cryobag) comprising human hepatocytes may include at least 1 million, at least 2 million, at least 5 million, at least 10 million, at least 20 million, at least 25 million, at least 50 million, at least 75 million, at least 100 million, at least 200 million, at least 250 million, at least 500 million, at least 750 million, at least 1 billion, at least 2 billion, at least 3 billion, at least 4 billion, at least 5 billion, at least 6 billion, at least 7 billion, at least 8 billion, at least 9 billion, at least 10 billion, or more human hepatocytes derived from a single donor, including e.g., where the single container is part of a plurality of similar, identical, or at least substantially similar containers that each contain the same or a substantially similar amount of the human hepatocytes. The number of individual containers in such a plurality may vary and may range from 10 or less to 10,000 or more, including e.g., from 10 to 10000, from 100 to 10000, from 250 to 10000, from 500 to 10000, from 750 to 10000, from 1000 to 10000, from 2000 to 10000, from 2500 to 10000, from 5000 to 10000, from 10 to 5000, from 100 to 5000, from 250 to 5000, from 500 to 5000, from 750 to 5000, from 1000 to 5000, from 2000 to 5000, from 10 to 1000, from 100 to 1000, from 250 to 1000, from 500 to 1000, from 1000 to 2000, from 1000 to 3000, from 1000 to 4000, from 2000 to 3000, from 2000 to 4000, from 2000 to 5000, and the like.

Also provided are isolated expanded populations of human hepatocytes, wherein a population is expanded from an initial population of human hepatocytes obtained from a human liver or a portion thereof; is isolated following expansion by a process that excludes centrifugal sedimentation; and displays improved cell fitness, as measured by one or more potency assays, as compared to a comparable human hepatocyte population isolated using centrifugal sedimentation. In certain embodiments, the isolated expanded population of human hepatocytes displays equivalent or improved cell fitness as compared to the initial population of human hepatocytes, as measured by one or more potency assays. According to some embodiments, the improved cell fitness is measured by an assay for attachment efficiency, ammonia detoxification, human albumin expression, A1AT expression, CYP3A4, or any combination thereof.

Aspects of the present disclosure further include an isolated expanded population of human hepatocytes, where the human hepatocytes exhibit in vivo function, such as but not limited to e.g., in vivo human albumin expression levels, greater than or equal to freshly isolated and/or freshly thawed cryopreserved cadaveric hepatocytes.

### Methods of Use

Aspects of the present disclosure further include methods of using the isolated expanded human hepatocytes and related compositions of the present disclosure.

According to some embodiments, provided are methods comprising administering an effective amount of the isolated expanded human hepatocytes of the present disclosure to an individual in need thereof. For example, the cell populations and/or hepatocytes can be used for the treatment of a subject for a condition where administration of an effective amount of the cells will have a desired therapeutic effect. In some instances, the desired therapeutic effect will be a result of one or more endogenous functions of the administered hepatocytes, including but not limited to e.g., hepatocyte metabolism, detoxification, synthesis of hepatocyte proteins (including e.g., albumin, fibrinogen, prothrombin, clotting factor (e.g., factor V, VII, IX, X, XI, and XII), protein C, protein S, antithrombin, lipoprotein, ceruloplasmin, transferrin, complement proteins, proteins of the hepatocyte proteome and/or secretome (such as e.g., those described in Franko et al. Nutrients. (2019) 11(8):1795; the disclosure of which is incorporated herein by reference in its entirety)), and the like. In some instances, the desired therapeutic effect will be a result of one or more heterologous functions of the administered hepatocytes, e.g., a heterologous function of a gene product encoded by a functionally integrated transgene.

Cell populations including hepatocytes as described herein can be used for treatment and/or prevention of any liver disease or disorder. For example, reconstitution of liver tissue in a patient by the introduction of hepatocytes is a potential therapeutic option for patients with any liver condition(s) (e.g., acute liver failure, chronic liver disease and/or metabolic or monogenic disease), including as a permanent treatment for these conditions by persistence of transplanted hepatocytes and/or repopulating the subject's liver with isolated expanded human hepatocytes as described herein. Hepatocyte reconstitution may be used, for example, to introduce isolated expanded human hepatocytes to replace hepatocytes lost as a result of disease, physical or chemical injury, or malignancy. In addition, isolated expanded human hepatocytes can be used to populate medical devices, such as e.g., artificial liver assist devices, decellularized scaffolds, such as e.g., decellularized liver scaffolds, and the like.

In some instances, the instant methods comprise transplantation, including e.g., orthotopic transplantation, of the isolated expanded human hepatocytes into a subject in need thereof. Human hepatocytes produced according to the methods described herein can be further purified, cryopreserved, and/or extensively characterized prior to transplantation or infusion. Among other uses, hepatocytes produced according to the methods described herein may provide on-demand therapy for patients with one or more severe liver diseases.

Cell populations and compositions comprising such cells as described herein can be administered to subjects by any suitable means and to any part, organ, or tissue of the subject. Non-limiting examples of administration means include portal vein infusion, umbilical vein infusion, direct splenic capsule injection, splenic artery infusion, infusion into the omental bursa and/or intraperitoneal injection (infusion, transplantation). In certain embodiments, the compositions comprise encapsulated hepatocytes that are transplanted by infusion into the intraperitoneal space and/or the omental bursa. In certain embodiments, the compositions comprise acellular/decellularized scaffold, including e.g., synthetic scaffolds, decellularized liver, and the like, that are seeded and/or repopulated with hepatocytes as described herein and surgically transplanted into a subject in need thereof.

In addition to or as an alternative to administration (transplantation) to a subject (patient), the hepatocytes as described herein can also be used for supplying hepatocytes to devices or compositions useful in treating subjects with liver disease. Non-limiting examples of such devices or compositions in which the hepatocytes of the present disclosure can be used include bioartificial livers (BAL) (extracorporeal supportive devices for subjects suffering from acute liver failure) and/or decellularized livers (recellularizing organ scaffolds to provide liver function in the subject). See, e.g., Shaheen et al. (2019) Nat Biomed Eng. doi: 10.1038/s41551-019-0460-x; Glorioso et al. (2015) J Hepatol 63(2):388-98.

Disease and disorders that may be treated using the methods and/or cell populations described herein include but are not limited to Crigler-Najjar syndrome type 1; familial hypercholesterolemia; Factor VII deficiency; Glycogen storage disease type I; infantile Refsum's disease; Progressive familial intrahepatic cholestasis type 2; hereditary tyrosinemia type 1; and various urea cycle defects; acute liver failure, including juvenile and adult patients with acute drug-induced liver failure; viral-induced acute liver failure; idiopathic acute liver failure; mushroom-poisoning-induced acute liver failure; post-surgery acute liver failure; acute liver failure induced by acute fatty liver of pregnancy; chronic liver disease, including cirrhosis and/or fibrosis; acute-on-chronic liver disease caused by one of the following acute events: alcohol consumption, drug ingestion, and/or hepatitis B flares. Thus, the patients may have one or more of these or other liver conditions.

In some instances, diseases and disorders treated according to the methods described herein may include hepatocyte-specific (hepatocyte-intrinsic) dysfunction. For example, the dysfunction, and the etiology of the disease and/or disorder, may be due to, or primarily attributable to, dysfunction of the endogenous hepatocytes present within the subject. In some instances, the hepatocyte-specific dysfunction may be genetic or inherited by the subject. In some instances, the etiology of the disease or disorder does not substantially involve cell types other than hepatocytes. In some instances, the disease or disorder results in decreased liver function, liver disease (acute or chronic), or other adverse condition derived from the endogenous hepatocytes. Accordingly, in some instances, e.g., where a disease is intrinsic to the endogenous hepatocyte population, an effective treatment may include replacement, supplementation, transplantation, or repopulation with hepatocytes as described herein.

Diseases and disorders characterized by hepatocyte-specific (hepatocyte-intrinsic) dysfunction may be contrasted with diseases and disorders having an etiology that is not hepatocyte specific and involve hepatocyte extrinsic factors. Examples of diseases having factors and/or an etiology that is hepatocyte extrinsic include but are not limited to e.g., alcoholic steatohepatitis, alcoholic liver disease (ALD), hepatic steatosis/nonalcoholic fatty liver disease (NAFLD), and the like.

Examples of hepatocyte-intrinsic and hepatocyte-related diseases include liver-related enzyme deficiencies, hepatocyte-related transport diseases, and the like. Such liver-related deficiencies may be acquired or inherited diseases and may include metabolic diseases (such as e.g. liver-based metabolic disorders). Inherited liver-based metabolic disorders may be referred to as “inherited metabolic diseases of the liver”, such as but not limited to e.g., those diseases described in Ishak, Clin Liver Dis (2002) 6:455-479. Liver-related deficiencies may, in some instances, result in acute and/or chronic liver disease, including e.g., where acute and/or chronic liver disease is a result of the deficiency when left untreated or insufficiently treated. Non-limiting examples of inherited liver-related enzyme deficiencies, hepatocyte-related transport diseases, and the like include Crigler-Najjar syndrome type 1; familial hypercholesterolemia, Factor VII deficiency, Glycogen storage disease type I, infantile Refsum's disease, Progressive familial intrahepatic cholestasis type 2, hereditary tyrosinemias (e.g., hereditary tyrosinemia type 1), genetic urea cycle defects, phenylketonuria (PKU), hereditary hemochromatosis, Alpha-I antitrypsin deficiency (AATD), Wilson Disease, and the like. Non-limiting examples of inherited metabolic diseases of the liver, including metabolic diseases having at least some liver phenotype, pathology, and/or liver-related symptom(s), include 5-beta-reductase deficiency, AACT deficiency, Aarskog syndrome, abetalipoproteinemia, adrenal leukodystrophy, Alpers disease, Alpers syndrome, alpha-1-antitrypsin deficiency, antithrombin III deficiency, arginase deficiency, argininosuccinic aciduria, arteriohepatic dysplasia, autoimmune lymphoproliferative syndrome, benign recurrent cholestasis, beta-thalassemia, Bloom syndrome, Budd-Chiari syndrome, carbohydrate-deficient glycoprotein syndrome, ceramidase deficiency, ceroid lipofuscinosis, cholesterol ester storage disease, cholesteryl ester storage disease, chronic granulomatous, chronic hepatitis C, Crigler-Najjar syndrome, cystic fibrosis, cystinosis, diabetes mellitus, Dubin-Johnson syndrome, endemic Tyrolean cirrhosis, erythropoietic protoporphyria, Fabry disease, familial hypercholesterolemia, familial steatohepatitis, fibrinogen storage disease, galactosemia, gangliosidosis, Gaucher disease, genetic hemochromatosis, glycogenosis type 1a, glycogenosis type 2, glycogenosis type 3, glycogenosis type 4, granulomatous disease, hepatic familial amyloidosis, hereditary fructose intolerance, hereditary spherocytosis, Hermansky-Pudlak syndrome, homocystinuria, hyperoxaluria, hypobetalipoproteinemia, hypofibrinogenemia, intrahepatic cholestasis of pregnancy, Lafora disease, lipoamide dehydrogenase deficiency, lipoprotein disorders, Mauriac syndrome, metachromatic leukodystrophy, mitochondrial cytopathies, Navajo neurohepatopathy, Niemann-Pick disease, nonsyndromic paucity of bile ducts, North American Indian childhood cirrhosis, ornithine transcarbamylase deficiency, partial lipodystrophy, Pearson syndrome, porphyria cutanea tarda, progressive familial intrahepatic cholestasis, progressive familial intrahepatic cholestasis type 1, progressive familial intrahepatic cholestasis type 2, protein C deficiency, Shwachman syndrome, Tangier disease, thrombocytopenic purpura, total lipodystrophy, type 1 glycogenosis, Tyrolean cirrhosis, tyrosinemia, urea cycle disorders, venocclusive disease, Wilson disease, Wolman disease, X-linked hyper-IgM syndrome, and Zellweger syndrome.

Treatment of subjects according to the methods described herein may result in various clinical benefits and/or measurable outcomes, including but not limited to e.g., prolonged survival, delayed disease progression (e.g., delayed liver failure), prevention of liver failure, improved and/or normalized liver function, improved and/or normalized amino acid levels, improved and/or normalized ammonia levels, improved and/or normalized albumin levels, improved and/or normalized bilirubin, recovery from a failure to thrive phenotype, reduction in lethargy, reduction in obtundation, reduction in seizures, reduction in jaundice, improved and/or normalized serum glucose, improved and/or normalized INR, improved and/or normalized urine test results, and the like.

For example, in some instances, administration of the isolated expanded human hepatocytes of the present disclosure results in at least a 5% increase in survival of subjects having a liver disease and/or a condition resulting in liver failure as compared to e.g., subjects treated according to the standard of care. The observed level of enhanced survival in such subject may vary and may range from an at least 5% to 60% or more increase, including but not limited to e.g., an at least 5%, at least 10%, at least 15%, at least 20%, at least 25%, at least 30%, at least 35%, at least 40%, at least 45%, at least 50%, at least 55%, at least 60% or more increase in survival. In some instances, subjects may experience a delay in disease progression and/or the onset of one or more disease symptoms, such as but not limited to e.g., liver failure and/or any symptom(s) attributable thereto. Such a delay in disease progression and/or symptom onset may last days, weeks, months or years, including but not limited to e.g., at least one week, at least one month, at least 2 months, at least 3 months, at least 4 months, at least 5 months, at least 6 months, at least a year or more. The hepatocytes as described herein administered to a patient effect a beneficial therapeutic response in the patient over time.

Non-limiting examples of liver conditions that may be treated include acute intermittent porphyria, acute liver failure, alagille syndrome, alcoholic fatty liver disease, alcoholic hepatitis, alcoholic liver cirrhosis, alcoholic liver disease, alpha 1-antitrypsin deficiency, amebic liver abscess, autoimmune hepatitis, biliary liver cirrhosis, budd-chiari syndrome, chemical and drug induced liver injury, cholestasis, chronic hepatitis, chronic hepatitis B, chronic hepatitis C, chronic hepatitis D, end stage liver disease, erythropoietic protoporphyria, fascioliasis, fatty liver disease, focal nodular hyperplasia, hepatic echinococcosis, hepatic encephalopathy, hepatic infarction, hepatic insufficiency, hepatic porphyrias, hepatic tuberculosis, hepatic veno-occlusive disease, hepatitis, hepatocellular carcinoma, hepatoerythropoietic porphyria, hepatolenticular degeneration, hepatomegaly, hepatopulmonary syndrome, hepatorenal syndrome, hereditary coproporphyria, liver abscess, liver cell adenoma, liver cirrhosis, liver failure, liver neoplasm, massive hepatic necrosis, non-alcoholic fatty liver disease, parasitic liver disease, peliosis hepatis, porphyria cutanea tarda, portal hypertension, pyogenic liver abscess, reye syndrome, variegate porphyria, viral hepatitis, viral hepatitis A, viral hepatitis B, viral hepatitis C, viral hepatitis D, viral hepatitis E, and zellweger syndrome, and the like. In some instances, a subject may be treated for fibrosis or a fibrotic condition. In some instances, a subject may be treated for cirrhosis or a cirrhotic condition.

Treatments described herein may be performed chronically (i.e., continuously) or non-chronically (i.e., non-continuously) and may include administration of one or more agents chronically (i.e., continuously) or non-chronically (i.e., non-continuously). Chronic administration of one or more agents according to the methods described herein may be employed in various instances, including e.g., where a subject has a chronic condition, including e.g., a chronic liver condition (e.g., chronic liver disease, cirrhosis, alcoholic liver disease, non-alcoholic fatty liver disease (NAFLD/NASH), chronic viral hepatitis, etc.), a chronic genetic liver condition (alpha-1 antitrypsin deficiency, Hereditary hemochromatosis, Wilson disease, etc.), chronic liver-related autoimmune conditions (e.g., primary biliary cirrhosis (PBC), primary sclerosing cholangitis (PSC), autoimmune hepatitis (AIH), etc.) etc. Administration of one or more agents for a chronic condition may include but is not limited to administration of the agent for multiple months, a year or more, multiple years, etc. Such chronic administration may be performed at any convenient and appropriate dosing schedule including but not limited to e.g., daily, twice daily, weekly, twice weekly, monthly, twice monthly, etc. In some instances, e.g., in the case of correction of a genetic condition or other persistent gene therapies, a chronic condition may be treated by a single or few (e.g., 2, 3, 4, or 5) treatments. Non-chronic administration of one or more agents may include but is not limited to e.g., administration for a month or less, including e.g., a period of weeks, a week, a period of days, a limited number of doses (e.g., less than 10 doses, e.g., 9 doses or less, 8 doses or less, 7 doses or less, etc., including a single dose).

An effective amount of a composition of therapeutic cells will depend, at least, on the particular method of use, the subject being treated, the severity of the affliction, the manner of administration of the composition, and the mechanism of action of the therapeutic cells. A “therapeutically effective amount” of a composition is a quantity of a specified reagent, e.g., therapeutic cells, sufficient to achieve a desired effect in a subject being treated.

The specific dose level and frequency of dosage for any particular subject may be varied and will depend upon a variety of factors, including the activity of the cells of the composition(s), the stability and length of action of the cells of the composition, the age, body weight, general health, sex and diet of the subject, mode and time of administration, drug combination(s) co-administered, and severity of the condition of the host undergoing therapy.

The above listed examples of therapies should not be construed as limiting and essentially any appropriate therapy resulting in the desired therapeutic outcome in subjects identified as described may be employed.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. For example, a preventative treatment, i.e. a prophylactic treatment, may include a treatment that effectively prevents a condition (e.g., a liver condition) or a treatment that effectively prevents or controls progression of a condition (e.g., a liver condition). In some instances, the treatment may result in a treatment response, such as a complete response or a partial response. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s).

Those in need of treatment can include those already afflicted (e.g., those with a condition, those with a liver condition (e.g., acute liver condition, chronic liver condition, etc.), those with cirrhosis, those with fibrosis, those with a disease, those with a monogenic disease, etc.) as well as those in which prevention is desired (e.g., those with increased susceptibility to a condition (e.g., a liver condition); those suspected of having a condition (e.g., a liver condition); those with an increased risk of developing a condition (e.g., a liver condition); those with increased environmental exposure to practices or agents causing a condition (e.g., a liver condition); those suspected of having a genetic or behavioral predisposition to a condition (e.g., a liver condition); those with a condition (e.g., a liver condition); those having results from screening indicating an increased risk of a condition (e.g., a liver condition); those having tested positive for a condition (e.g., a liver condition); those having tested positive for one or more biomarkers of a condition (e.g., a liver condition), etc.).

A therapeutic treatment is one in which the subject is afflicted prior to administration and a prophylactic treatment is one in which the subject is not afflicted prior to administration. In some embodiments, the subject has an increased likelihood of becoming afflicted or is suspected of having an increased likelihood of becoming afflicted (e.g., relative to a standard, e.g., relative to the average individual, e.g., a subject may have a genetic predisposition to a condition and/or a family history indicating increased risk), in which case the treatment can be a prophylactic treatment.

Notwithstanding the appended claims, the present disclosure is also defined by the following embodiments.

1. A method of producing isolated expanded human hepatocytes, the method comprising:
collecting hepatocytes from the liver of a non-human in vivo bioreactor in which human hepatocytes have been expanded, wherein the collected hepatocytes comprise a xenomixture of the expanded human hepatocytes and non-human in vivo bioreactor hepatocytes; and
A) subjecting the xenomixture to centrifugal elutriation and then removing non-human in vivo bioreactor cells from the elutriated xenomixture via a negative selection process to produce isolated expanded human hepatocytes; or
B) removing non-human in vivo bioreactor cells via a negative selection process and then performing centrifugal elutriation to produce isolated expanded human hepatocytes.
   2. A method of producing isolated expanded human hepatocytes, the method comprising:
introducing human hepatocytes into the liver of a non-human in vivo bioreactor;
expanding the human hepatocytes in the liver of the non-human in vivo bioreactor;
collecting hepatocytes from the liver of the non-human in vivo bioreactor, wherein the collected hepatocytes comprise a xenomixture of expanded human hepatocytes and non-human in vivo bioreactor hepatocytes;
subjecting the xenomixture to centrifugal elutriation under conditions sufficient to produce an elutriation fraction enriched for the expanded human hepatocytes; and
removing non-human in vivo bioreactor cells from the elutriation fraction via a negative selection process to produce isolated expanded human hepatocytes.
   3. The method according to embodiment 2, wherein introducing the human hepatocytes into the liver of the non-human in vivo bioreactor comprises delivering the human hepatocytes to the spleen of the non-human in vivo bioreactor.
   4. The method according to embodiment 3, wherein delivering the human hepatocytes to the spleen of the non-human in vivo bioreactor is by splenic injection.
   5. The method according to any of the preceding embodiments, comprising monitoring the expansion of the human hepatocytes in the liver of the non-human in vivo bioreactor.
   6. The method according to embodiment 5, wherein the monitoring comprises monitoring the level of a circulating biomarker secreted by the human hepatocytes in the non-human in vivo bioreactor during the expanding.
   7. The method according to embodiment 6, wherein the circulating biomarker is human albumin (hAlb).
   8. The method according to embodiment 6 or embodiment 7, wherein the level of the circulating biomarker is monitored in whole blood obtained from the non-human in vivo bioreactor.
   9. The method according to any one of embodiments 5 to 8, wherein collecting hepatocytes from the liver of the non-human in vivo bioreactor commences based on the monitored level of the circulating biomarker reaching a threshold level.
   10. The method according to any one of embodiments 1 to 9, wherein collecting hepatocytes from the liver of the non-human in vivo bioreactor commences based on a clinical score cutoff being met.
   11. The method according to any one of embodiments 1 to 10, wherein the expanded human hepatocytes constitute 50% or greater, 60% or greater, or 70% or greater of the total cells present in the elutriation fraction.
   12. The method according to any one of embodiments 1 to 11, wherein the non-human in vivo bioreactor is deficient for fumarylacetoacetate hydrolase (Fah).
   13. The method according to embodiment 12, wherein expanding comprises 2-(2-nitro-4-trifluoromethylbenzoyl)-1,3-cyclohexanedione (NTBC) cycling.
   14. The method according to any one of embodiments 1 to 13, wherein the non-human in vivo bioreactor is a rodent in vivo bioreactor.
   15. The method according to embodiment 14, wherein the rodent in vivo bioreactor is a rat in vivo bioreactor.
   16. The method according to embodiment 14 or embodiment 15, wherein the rodent in vivo bioreactor is deficient for interleukin 2 receptor subunit gamma (IL2rg), recombination activating gene 1 (RAG1), recombination activating gene 2 (RAG2), or a combination thereof.
   17. The method according to any one of embodiments 1 to 13, wherein the non-human in vivo bioreactor is a pig in vivo bioreactor.
   18. The method according to any one of embodiments 1 to 17, wherein the negative selection process is an antibody-based negative selection process.
   19. The method according to embodiment 18, wherein the antibody-based negative selection process comprises:
contacting the elutriation fraction, the elutriated xenomixture, or the xenomixture with a primary antibody specific for non-human in vivo bioreactor cells under conditions sufficient for specific binding of the primary antibody to non-human in vivo bioreactor cells present in the elutriation fraction, elutriated xenomixture, or xenomixture; and
removing non-human in vivo bioreactor cells from the elutriation fraction, elutriated xenomixture, or xenomixture utilizing the primary antibody.
   20. The method according to embodiment 19, wherein removing non-human in vivo bioreactor cells utilizing the primary antibody comprises contacting the primary antibody with a labeled secondary antibody under conditions sufficient for binding of the secondary antibody to the primary antibody, and utilizing the label of the labeled secondary antibody to remove, from the elutriation fraction, elutriated xenomixture, or xenomixture, complexes comprising labeled secondary antibody, primary antibody, and a non-human in vivo bioreactor cell.
   21. The method according to embodiment 19, wherein the primary antibody is labeled, and wherein removing non-human in vivo bioreactor cells comprises utilizing the label to remove, from the elutriation fraction, elutriated xenomixture, or xenomixture, complexes comprising primary antibody and a non-human in vivo bioreactor cell.
   22. The method according to embodiment 20 or embodiment 21, wherein the label comprises an affinity tag.
   23. The method according to embodiment 20 or embodiment 21, wherein the label is magnetically responsive.
   24. The method according to embodiment 23, wherein the label comprises a magnetic bead.
   25. The method according to any one of embodiments 19 to 24, wherein the primary antibody is a pan-non-human in vivo bioreactor antibody.
   26. The method according to embodiment 25, wherein the pan-non-human in vivo bioreactor antibody is an anti-histocompatibility antigen antibody.
   27. The method according to embodiment 26, wherein the non-human in vivo bioreactor is a rat in vivo bioreactor.
   28. The method according to embodiment 27, wherein the anti-histocompatibility antigen antibody is an anti-RT1-region, class I (A) (RT1A) antibody.
   29. The method according to embodiment 28, wherein the anti-RT1A antibody competes for binding to RT1A with an antibody comprising:
a variable heavy chain (V_{H}) polypeptide comprising:
   a V_{H} CDR1 comprising the amino acid sequence GDSITSGY (SEQ ID NO:1),
   a V_{H} CDR2 comprising the amino acid sequence ISYSGST (SEQ ID NO:2), and
   a V_{H} CDR3 comprising the amino acid sequence ASHSHWYFDV (SEQ ID NO:3), and
a variable light chain (V_{L}) polypeptide comprising:
   a V_{L} CDR1 comprising the amino acid sequence QDISNY (SEQ ID NO:4),
   a V_{L} CDR2 comprising the amino acid sequence YTS (SEQ ID NO:5), and
   a V_{L} CDR3 comprising the amino acid sequence QQGNTLPWT (SEQ ID NO:6),
wherein CDRs are defined according to IMGT.
   30. The method according to embodiment 28, wherein the anti-RT1A antibody comprises:
a variable heavy chain (V_{H}) polypeptide comprising
   a V_{H} CDR1 comprising the amino acid sequence GDSITSGY (SEQ ID NO:1),
   a V_{H} CDR2 comprising the amino acid sequence ISYSGST (SEQ ID NO:2), and
   a V_{H} CDR3 comprising the amino acid sequence ASHSHWYFDV (SEQ ID NO:3), and TT comprising
   a V_{L} CDR1 comprising the amino acid sequence QDISNY (SEQ ID NO:4),
   a V_{L} CDR2 comprising the amino acid sequence YTS (SEQ ID NO:5), and
   a V_{L} CDR3 comprising the amino acid sequence QQGNTLPWT (SEQ ID NO:6).
      31. The method according to embodiment 29 or embodiment 30, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:15; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:18.
   32. The method according to any one of embodiments 1 to 31, wherein the method does not comprise a step of centrifugal sedimentation to enrich for expanded human hepatocytes.
   33. The method according to embodiment 32, wherein the isolated expanded human hepatocytes exhibit improved cell fitness as compared to a comparable human hepatocyte population isolated using centrifugal sedimentation.
   34. The method according to any one of embodiments 1 to 33, wherein the isolated expanded human hepatocytes exhibit equivalent or improved cell fitness as compared to the human hepatocytes introduced into the liver of a non-human in vivo bioreactor.
   35. The method according to any one of embodiments 32 to 34, wherein the isolated expanded human hepatocytes exhibit equivalent or improved cell fitness as compared to a comparable previously cryopreserved, freshly thawed human cadaveric hepatocyte population.
   36. The method according to any one of embodiments 33 to 35, wherein the improved cell fitness is measured by an assay for attachment efficiency, ammonia detoxification, human albumin expression, A1AT expression, CYP3A4, or any combination thereof.
   37. The method according to embodiment 36, wherein the improved cell fitness is measured by an in vivo human albumin assay.
   38. The method according to any one of embodiments 1 to 37, comprising:
introducing human hepatocytes into the livers of a plurality of non-human in vivo bioreactors;
expanding the human hepatocytes in the livers of the non-human in vivo bioreactors;
collecting hepatocytes from the livers of the non-human in vivo bioreactors, wherein the collected hepatocytes comprise a xenomixture of expanded human hepatocytes and non-human in vivo bioreactor hepatocytes; and
subjecting the xenomixture to centrifugal elutriation under conditions sufficient to produce an elutriation fraction enriched for the expanded human hepatocytes and removing non-human in vivo bioreactor cells from the elutriation fraction via a negative selection process to produce isolated expanded human hepatocytes; or
removing non-human in vivo bioreactor cells from the xenomixture via a negative selection process and then subjecting the xenomixture to centrifugal elutriation under conditions sufficient to produce an elutriation fraction enriched for the expanded human hepatocytes to produce isolated expanded human hepatocytes.
   39. The method according to embodiment 38, wherein the method comprises pooling the hepatocytes collected from the livers of the non-human in vivo bioreactors during the collecting, after the collecting, before the elutriation, during the elutriation, after the elutriation, before the negative selection process, during the negative selection process, or after the negative selection process.
   40. The method according to any one of embodiments 1 to 39, wherein the human hepatocytes are derived from a single human donor.
   41. Isolated expanded human hepatocytes produced according to the method of any one of embodiments 1 to 40.
   42. The isolated expanded human hepatocytes of embodiment 41, wherein the isolated expanded human hepatocytes are cryopreserved.
   43. The isolated expanded human hepatocytes of embodiment 41 or embodiment 42, wherein the isolated expanded human hepatocytes are derived from a single human donor.
   44. A population of at least 1 billion of the isolated expanded human hepatocytes of any one of embodiments 41 to 43, optionally wherein the population is present in a single container.
   45. A method comprising administering an effective amount of the isolated expanded human hepatocytes of any one of embodiments 41 or embodiment 44 to an individual in need thereof.
   46. The method according to embodiment 45, wherein the individual in need thereof has acute liver failure, alcoholic liver disease, chronic liver disease, acute-on-chronic liver disease, liver fibrosis, liver cirrhosis, hepatic encephalopathy, hepatitis, or a combination thereof.
   47. A method of enriching for human hepatocytes in a xenomixture, the method comprising:
subjecting a xenomixture comprising human hepatocytes and at least one type of non-human hepatocytes to centrifugal elutriation under conditions sufficient to produce an elutriation fraction enriched for the human hepatocytes.
   48. The method according to embodiment 47, wherein the non-human hepatocytes are deficient for fumarylacetoacetate hydrolase (Fah).
   49. The method according to embodiment 47 or embodiment 48, wherein the xenomixture comprises rodent hepatocytes.
   50. The method according to embodiment 49, wherein the xenomixture comprises rat hepatocytes.
   51. The method according to embodiment 49 or embodiment 50, wherein the rodent hepatocytes are deficient for interleukin 2 receptor subunit gamma (IL2rg), a recombination activating gene 1 (RAG1), a recombination activating gene 2 (RAG2), or a combination thereof.
   52. A method of enriching for human hepatocytes in a xenomixture, the method comprising subjecting a xenomixture comprising human hepatocytes and non-human hepatocytes to an antibody-based negative selection process.
   53. The method according to embodiment 52, wherein the xenomixture is produced from the liver of a in vivo bioreactor comprising the human hepatocytes and non-human hepatocytes.
   54. The method according to embodiment 52 or embodiment 53, wherein the antibody-based negative selection process comprises:
contacting the xenomixture with a primary antibody specific for the non-human hepatocytes under conditions sufficient for specific binding of the primary antibody to the non-human hepatocytes; and
removing the non-human hepatocytes from the xenomixture utilizing the primary antibody.
   55. The method according to embodiment 54, wherein removing the non-human hepatocytes from the xenomixture utilizing the primary antibody comprises contacting the antibody with a labeled secondary antibody under conditions sufficient for binding of the secondary antibody to the primary antibody, and utilizing the label of the labeled secondary antibody to remove from the xenomixture complexes comprising the labeled secondary antibody, the primary antibody, and the non-human hepatocyte.
   56. The method according to embodiment 54, wherein the primary antibody is labeled, and wherein removing the non-human hepatocytes from the xenomixture comprises utilizing the label to remove from the xenomixture complexes comprising the primary antibody and the non-human hepatocyte.
   57. The method according to embodiment 55 or embodiment 56, wherein the label comprises an affinity tag.
   58. The method according to any one of embodiments 55 to 57, wherein the label is magnetically responsive.
   59. The method according to embodiment 58, wherein the label comprises a magnetic bead.
   60. The method according to any one of embodiments 54 to 59, wherein the antibody specific for the non-human hepatocytes is a pan-non-human antibody.
   61. The method according to embodiment 60, wherein the pan-non-human antibody is an anti-histocompatibility antigen antibody.
   62. The method according to embodiment 61, wherein the non-human hepatocytes are rat hepatocytes.
   63. The method according to embodiment 62, wherein the anti-histocompatibility antigen antibody is an anti-RT1-region, class I (A) (RT1A) antibody.
   64. The method according to embodiment 63, wherein the anti-RT1A antibody competes for binding to RT1A with an antibody comprising:
a variable heavy chain (V_{H}) polypeptide comprising:
   a V_{H} CDR1 comprising the amino acid sequence GDSITSGY (SEQ ID NO:1),
   a V_{H} CDR2 comprising the amino acid sequence ISYSGST (SEQ ID NO:2), and
   a V_{H} CDR3 comprising the amino acid sequence ASHSHWYFDV (SEQ ID NO:3), and
a variable light chain (V_{L}) polypeptide comprising:
   a V_{L} CDR1 comprising the amino acid sequence QDISNY (SEQ ID NO:4),
   a V_{L} CDR2 comprising the amino acid sequence YTS (SEQ ID NO:5), and
   a V_{L} CDR3 comprising the amino acid sequence QQGNTLPWT (SEQ ID NO:6),
wherein CDRs are defined according to IMGT.
   65. The method according to embodiment 63, wherein the anti-RT1A antibody comprises:
a variable heavy chain (V_{H}) polypeptide comprising:
   a V_{H} CDR1 comprising the amino acid sequence GDSITSGY (SEQ ID NO:1),
   a V_{H} CDR2 comprising the amino acid sequence ISYSGST (SEQ ID NO:2), and
   a V_{H} CDR3 comprising the amino acid sequence ASHSHWYFDV (SEQ ID NO:3), and
a variable light chain (V_{L}) polypeptide comprising:
   a V_{L} CDR1 comprising the amino acid sequence QDISNY (SEQ ID NO:4),
   a V_{L} CDR2 comprising the amino acid sequence YTS (SEQ ID NO:5), and
   a V_{L} CDR3 comprising the amino acid sequence QQGNTLPWT (SEQ ID NO:6).
      66. The method according to embodiment 64 or embodiment 65, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:15; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:18.
   67. A composition comprising a cell population derived from a xenomixture, the xenomixture comprising dissociated human hepatocytes and at least one type of non-human hepatocytes, the cell population comprising at least 60% human hepatocytes, optionally wherein the composition comprises 40% or less non-human hepatocytes.
   68. A composition comprising isolated expanded human hepatocytes produced according to the method of any one of embodiments 1 to 40.
   69. A pharmaceutical preparation suitable for delivery to a human subject, the pharmaceutical preparation comprising the composition of embodiment 67 or embodiment 68 and at least 1 billion of the human hepatocytes.
   70. The pharmaceutical preparation according to embodiment 69, wherein the at least 1 billion hepatocytes are derived from a single human donor.
   71. An isolated expanded population of human hepatocytes, wherein the population:
is expanded from an initial population of human hepatocytes obtained from a human liver or a portion thereof;
is isolated following expansion by a process that excludes centrifugal sedimentation; and
displays improved cell fitness, as measured by one or more potency assays, as compared to a comparable human hepatocyte population isolated using centrifugal sedimentation.
   72. The population of human hepatocytes of embodiment 71, wherein the isolated expanded population of human hepatocytes displays equivalent or improved cell fitness as compared to the initial population of human hepatocytes, as measured by one or more potency assays.
   73. The population of human hepatocytes of embodiment 71 or embodiment 72, wherein the improved cell fitness is measured by an assay for attachment efficiency, ammonia detoxification, human albumin expression, A1AT expression, CYP3A4, or any combination thereof.
   74. An isolated expanded population of human hepatocytes, wherein the human hepatocytes exhibit:
in vivo human albumin expression levels greater than or equal to freshly isolated and/or cryopreserved cadaveric hepatocytes;
a reduced amount of immune cells and/or inflammatory cytokines as compared to freshly isolated and/or cryopreserved cadaveric hepatocytes, optionally wherein the inflammatory cytokines are selected from IL-1-beta, IL-6, TNF-alpha, and TGF-beta and/or the immune cells are selected from IL-1-beta-, IL-6-TNF-alpha-, and TGF-beta-expressing immune cells; or
a combination thereof.
   75. An isolated nucleic acid comprising one or more coding sequences encoding a variable heavy chain (V_{H}) polypeptide and/or a variable light chain (V_{L}) polypeptide of an anti-RT1A antibody, wherein the one or more coding sequences are mammalian codon optimized.
   76. The isolated nucleic acid of embodiment 75, wherein the anti-RT1A antibody comprises or competes with for binding to RT1A with an antibody comprising:
a variable heavy chain (V_{H}) polypeptide comprising:
   a V_{H} CDR1 comprising the amino acid sequence GDSITSGY (SEQ ID NO:1),
   a V_{H} CDR2 comprising the amino acid sequence ISYSGST (SEQ ID NO:2), and
   a V_{H} CDR3 comprising the amino acid sequence ASHSHWYFDV (SEQ ID NO:3), and
a variable light chain (V_{L}) polypeptide comprising:
   a V_{L} CDR1 comprising the amino acid sequence QDISNY (SEQ ID NO:4),
   a V_{L} CDR2 comprising the amino acid sequence YTS (SEQ ID NO:5), and
      a V_{L} CDR3 comprising the amino acid sequence QQGNTLPWT (SEQ ID NO:6).
      77. The isolated nucleic acid of embodiment 75 or embodiment 76, wherein the anti-RT1A antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:15; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:18.
   78. The isolated nucleic acid of any one of embodiments 75 to 77, wherein the one or more coding sequences comprise:
a sequence having 70% or greater, 75% or greater, 80% or greater, 85% or greater, 90% or greater, 95% or greater, 98% or greater, 99% or greater, or 100% identity to the nucleic acid sequence set forth in SEQ ID NO:14;
a sequence having 70% or greater, 75% or greater, 80% or greater, 85% or greater, 90% or greater, 95% or greater, 98% or greater, 99% or greater, or 100% identity to the nucleic acid sequence set forth in SEQ ID NO:17; or
a combination thereof.
   79. An expression vector comprising the isolated nucleic acid of any one of embodiments 75 to 78.
   80. An isolated expanded population of human hepatocytes having a gene signature comprising:
elevated expression of two or more, three or more, or four or more genes selected from Table 4;
reduced expression of two or more, three or more, or four or more genes selected from Table 5; or
elevated expression of at least one gene selected from Table 4 and reduced expression of at least one gene selected from Table 5,
optionally wherein the elevated and/or reduced expression is determined by comparison to corresponding gene expression in a reference primary human hepatocyte population.
   81. The isolated expanded population of human hepatocytes of embodiment 80, wherein the gene signature comprises:
elevated expression of two or more, three or more, or four or more genes selected from the group consisting of: GPC3, AKR1B10, FXYD2, PEG10, CYP7A1, and NQO1;
reduced expression of two or more, three or more, or four or more genes selected from the group consisting of: C9, SAA1, SAA2, CRP, NNMT, SPINK1, PLA2G2A, and ORM1; or
elevated expression of at least one gene selected from the group consisting of GPC3, AKR1B10, FXYD2, PEG10, CYP7A1, and NQO1 and reduced expression of at least one gene selected from the group consisting of C9, SAA1, SAA2, CRP, NNMT, SPINK1, PLA2G2A, and ORM1.
   82. The isolated expanded population of human hepatocytes of embodiment 80 or embodiment 81, wherein the elevated expression comprises an at least 2-fold elevation, as compared to corresponding expression in primary human hepatocytes, of each of the elevated genes of the gene signature and the reduced expression comprises an at least 2-fold reduction, as compared to corresponding expression in primary human hepatocytes, of each of the reduced genes of the gene signature.
   83. The isolated expanded population of human hepatocytes of any one of embodiments 80 to 82, wherein the isolated expanded human hepatocytes of the population are derived from a single human donor.
   84. The isolated expanded population of human hepatocytes of any one of embodiments 80 to 83, wherein the population comprises at least 1 billion of the isolated expanded human hepatocytes, optionally wherein the population is present in a single container.
   85. The isolated expanded population of human hepatocytes of any one of embodiments 80 to 84, wherein the population cryopreserved.
   86. A pharmaceutical preparation suitable for delivery to a human subject, the pharmaceutical preparation comprising the isolated expanded population of human hepatocytes of any one of embodiments 80 to 85.
   87. A method comprising administering an effective amount of the population of isolated expanded human hepatocytes of any one of embodiments 80 to 84 or pharmaceutical preparation embodiment 86 to an individual in need thereof.
   88. The method according to embodiment 87, wherein the individual in need thereof has acute liver failure, alcoholic liver disease, chronic liver disease, acute-on-chronic liver disease, liver fibrosis, liver cirrhosis, hepatic encephalopathy, hepatitis, or a combination thereof.

The following examples are offered by way of illustration and not by way of limitation.

### EXPERIMENTAL

### Example 1—Collection of Hepatocytes by Cadaveric Human Liver Perfusion

A donor human liver unsuitable or not needed for orthotopic transplantation was obtained from an organ procurement organization. Donor characteristics, medical history, as well as the appearance and history of the organ, was screened and, since acceptable criteria was reached, the organ and necessary reagents were prepared for hepatocyte collection by liver perfusion. Useful reagent solutions, including liver perfusion solution (LPS) I, LPS II, collagenase MA solution, BP protease (Bacillus polymyxa) solution, elutriation buffer (EB), and cryopreservation media, were prepared in advance and sterile filtered where applicable.

Sterile packaging was removed from the received liver. The lobes of the liver were resected, and the resected lobes were prepared for perfusion, however, in some instances, depending on liver size, the liver may be perfused whole. Visible vessels were flushed, and vessels were selected for perfusion. Plastic connectors were inserted into the selected vessels and secured in place. Cut surfaces of the liver lobes were sealed with medical grade adhesive and any unused large vessels present in the cut surface were closed. Using sterile tubing, peristaltic pumps were connected to the plastic connectors that have been inserted into the chosen vessels.

Using the peristaltic pumps, LPS I was pumped into the liver, followed by LPS II, and the flow rate was continually monitored and adjusted as needed during the perfusion of each solution. Once parenchymal breakdown was observed, the liver sections were disconnected from the pumps and mechanically dissociated into EB in a sterile collection container. The dissociated liver was then filtered to obtain a thoroughly mixed cell suspension. Cell counts and viability measurements were taken, and the cell suspensions were pooled and prepared for hepatocyte enrichment.

### Example 2—Closed System Hepatocyte Enrichment

A container of filtered cell suspension, prepared as described in Example 1, was connected to a container of EB, a hepatocyte collection container, and a waste collection container using sterile tubing and a tube welder. The tubing, now connected to the various containers, was fitted into the fluid flow control area of an elutriator. Using the elutriator, all tubing lines were primed with EB and then a cell bed was formed within the elutriation chamber using the cell suspension. Hepatocytes within the cell suspension were retained within the chamber while other cell types were removed. The remaining hepatocyte fraction was washed, eluted and collected into the sterile hepatocyte collection container, all within the closed system. Collection was continued until the initial container containing the filtered cell suspension was emptied.

By this method undesired cell types and debris were removed, allowing for the collection and rapid enrichment of human hepatocytes in a closed, sterile system without exposure of the hepatocytes to non-sterile conditions or reagents, such as percoll, or processes, such as repeated centrifugal sedimentation, that can be detrimental to human hepatocytes.

Next, cell counts and viability measurements were taken and the enriched hepatocytes were prepared for cryopreservation.

### Example 3—Cryopreservation of Enriched Freshly Isolated Human Hepatocytes

Enriched hepatocyte cell suspension was aliquoted into vessels for pelleting such as, e.g., 750 million cells per 225 mL centrifuge tube or 1.75 billion cells per 500 mL centrifuge tube, and the hepatocytes were pelleted by centrifugation. Cell pellets were gently resuspended in cryopreservation media under cold conditions to reach a final concentration of 10 million live cells per mL and the resuspended cells were kept at 4-8 deg. C. Hepatocytes prepared for cryopreservation were aliquoted into freezing containers such as, e.g., cryovials or cryobags, and the filled freezing containers were frozen using a controlled rate freezer using a hepatocyte specific program. After controlled rate freezing was complete, cryopreserved hepatocytes were transferred to vapor phase liquid nitrogen for storage.

### Example 4—In Vivo Bioreactor Urokinase Pre-Conditioning

In this example, in vivo bioreactor animals, e.g., fumarylacetoacetate hydrolase deficient (Fah−/−), IL2rg deficient (IL2rg−/−), and Rag1 or Rag2 deficient (Rag1−/− or Rag2−/−) rats, were preconditioned for engraftment of transplanted human hepatocytes by treatment with adenovirus-vectorized urokinase-type Plasminogen Activator (uPA). Human adenovirus type 5 (E1 deleted or E1/E3 deleted or similar) containing recombinant human uPA coding sequence in storage buffer was diluted with saline to generate a 2.5-5E10 pfu/mL viral stock, sterile filtered, and loaded into a sterile syringe with a half inch 29G needle for each rat to be preconditioned. Injection volume for each rat was calculated based on the previously determined titer of the relevant virus lot and the animal's body weight. One injection was delivered to each rat intravenously 24±2 hours before hepatocyte transplantation.

### Example 5—Preparation of Human Hepatocytes for Delivery into In Vivo Bioreactor

In this example, cryopreserved human hepatocytes were prepared for delivery into in vivo bioreactor animals, e.g., Fah−/−, IL2rg−/−, and Rag1−/− or Rag2−/− rats. A sufficient amount of hepatocytes, e.g., 5E6 cells/100 g of BW per rat, were retrieved from cryo-storage and kept on dry ice and then thawed quickly in a water bath. Where appropriate, the contents of multiple cryovials were pooled. Thawed cell suspensions were diluted with cell media, pelleted by centrifugation, washed, counted, and brought to a cell concentration of 25E06 viable cells/mL in cell media for injection. Aliquots of the prepared cell suspension were retained for analyses, including e.g., plating on collagen-coated wells/plates for morphology, plating density, and attachment analyses.

### Example 6—Delivery by Laparotomy Splenic Injection of Cryopreserved Cadaveric Human Hepatocytes to In Vivo Bioreactors for Expansion

Direct injection of hepatocytes into the liver was found to have certain undesirable characteristics, including e.g., decreased engraftment and hyper-localized engraftment, in certain instances. Accordingly, alternative delivery methods that provide for more systemic delivery were investigated. Whole-body systemic delivery, e.g., via retro-orbital injection, was deemed less desirable as compared to organ-systemic delivery methods.

All blood that enters the spleen travels immediately to and throughout the liver and anything of the appropriate size and viscosity injected into the spleen will travel immediately to the liver and disperse through all lobes. Moreover, the spleen is large enough to easily locate and inject, while also allowing for easy control of any bleeding that may occur. Thus, splenic delivery was chosen as the route for organ-systemic delivery to the liver.

Animal bioreactors were chosen for laparotomy splenic injection based on general health and the state of preconditioning. Aseptic technique and appropriate anesthesia and analgesics were employed. The surgical site was shaved, prepared, and cleaned. A 1 cm vertical incision was made in the skin, approximately 5 mm distal to the last rib, and the skin was gently separated from the muscle wall. An approximately 1 cm vertical incision was then made in the muscle wall. The spleen was maneuvered out of the peritoneal cavity and injection of hepatocytes was performed slowly and smoothly using a preloaded 29G syringe inserted at a low angle into the distal quarter of the spleen with injection being performed from the tail to the head of the spleen direction. Injection of the in situ spleen was performed smoothly and slowly with a preloaded 29G syringe inserted at a low angle into the middle third of the spleen with injection being performed from the head of the spleen towards the tail direction. Following full volume injection, the puncture site was covered to prevent backflow, the surgical opening was closed with a combination suture and wound clips or tissue adhesive, and the animal was allowed to recover under careful monitoring.

### Example 7—Delivery by Percutaneous Splenic Injection of Cryopreserved Cadaveric Human Hepatocytes to In Vivo Bioreactors for Expansion

Splenic delivery was chosen as the route for organ-systemic delivery to the liver for the reasons discussed above. The non-surgical approach of percutaneous or transdermal injection may be advantageous as it is less invasive, requires less consumables and may be more efficient if optimized. A non-surgical approach, once refined, would offer improved animal welfare and reduced risks of infection as the peritoneal cavity will not have been opened or exposed.

Animal bioreactors were chosen for percutaneous splenic injection based on general health and the state of preconditioning. Aseptic technique and appropriate anesthesia and analgesics were employed. The surgical site was shaved, prepared, and cleaned. Human hepatocytes for delivery were loaded into a 28G syringe. The spleen was identified through the skin by palpating the upper left quadrant of the abdomen and then gently grasped and immobilized against the muscle wall. The loaded syringe was inserted through the skin, muscle, and into the center of the spleen, moving distally into the organ. The cell suspension was slowly and smoothly injected into the spleen and intrasplenic injection was verified by minimal but noticeable swelling of the spleen. Following full volume injection, the fluid pressure was allowed to stabilize, the syringe was removed, and the animal was allowed to recover under careful monitoring.

### Example 8—Expansion of Human Hepatocytes and Quantitation of Human Albumin (hAlb) Levels in Human Hepatocyte Transplanted In Vivo Bioreactors

Fumarylacetoacetate hydrolase deficient (Fah−/−), IL2rg deficient (IL2rg−/−), and Rag1-deficient (Rag1−/−) rat (“FRG rat”) bioreactors administered cryopreserved cadaveric human hepatocytes, essentially as described in Example 6 or Example 7, were subsequently subjected to 2-(2-nitro-4-trifluoromethylbenzoyl)-1,3-cyclohexanedione (NTBC) cycling to both maintain animal health and promote engraftment and expansion of the received human hepatocytes. In some instances, animals were monitored for liver function and other indicators of health, such as but not limited to body weight, total bilirubin (TBIL), gamma-glutamyl transferase (GGT), glucose, total protein, albumin, aspartate aminotransferase (AST), alkaline phosphatase (ALP), alanine aminotransferase (ALT), and the like. In some cases, animals were assessed and assigned a veterinary clinical score at the time of assessment, including e.g., where the clinical score included assessments of body condition (e.g., fat, muscle, etc.), observation and scoring of animal behavior, body weight, and hydration status.

To assess the degree of human hepatocyte repopulation of in vivo bioreactor host liver with engrafted human hepatocytes, an ELISA for measuring hAlb levels in bioreactor whole blood was employed. After coating the ELISA plates with coating antibody solution, the plates were washed with ELISA wash buffer, blocked with ELISA blocking solution, and washed again with wash buffer. A standard curve was established using human reference serum and bioreactor whole blood samples were assessed on the same plate, which reactions were read on a plate reader. The concentrations of hAlb in each rat were determined in this way and, for this example, bioreactor animals having whole blood hAlb levels of at least 2500 micrograms per mL were considered to have livers sufficiently repopulated with human hepatocytes, and to have human hepatocyte populations that are sufficiently expanded, to advance to hepatocyte collection. In some instances, in addition to a human albumin threshold, a clinical score cutoff was also employed, such as e.g., a clinical score cutoff of at least 2.0, at least 2.5, at least 3.0, or at least 3.5, in addition to whole blood human albumin of at least 2500 micrograms per mL.

### Example 9—Screening to Identify Rat-Specific Antibodies and Purification of Human Hepatocytes from a Hepatocyte Xenomixture by Negative Selection of Non-Human Hepatocytes

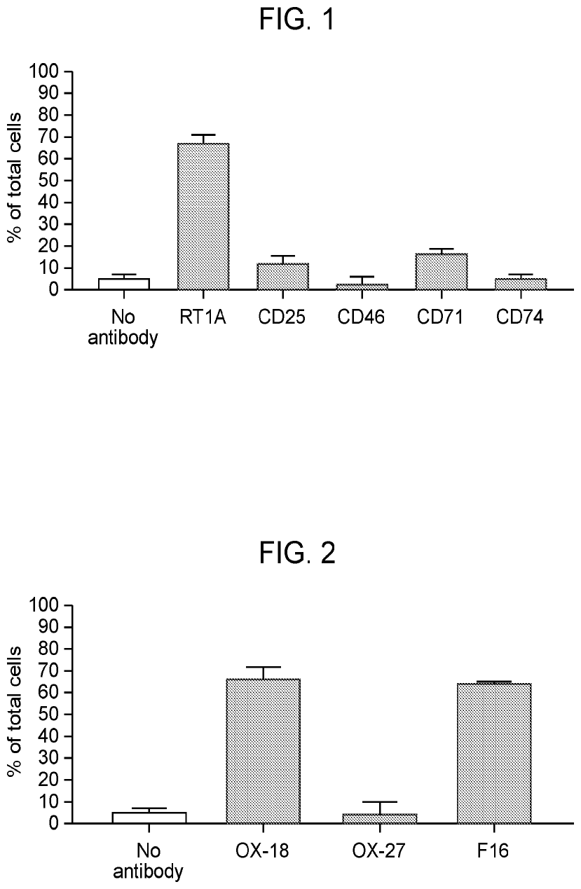

Screening was performed to identify a rat-specific antibody suitable for use in negative selection to purify human hepatocytes from a xenomixture of human hepatocytes and rat cells, as is obtained from perfusion of humanized rat bioreactor livers. Numerous candidate target antigens were evaluated for pan-binding of rat cells by antibodies targeting each candidate antigen. For example, FIG. 1 displays the percent of total rat cells bound by antibodies to each of a subset of rat antigens evaluated, including e.g., rat RT1A class I histocompatibility antigen (“RT1A”, see e.g., UniProtKB P16391 (SEQ ID NO:7), GenBank: AAB49324.1), rat dipeptidyl peptidase 4 (“CD26”, see e.g., UniProtKB P14740 (SEQ ID NO:8), GenBank: AAA41096.1 (SEQ ID NO:9), rat membrane cofactor protein (“CD46”, see e.g., UniProtKB Q9ZOM4 (SEQ ID NO:10), RefSeq: NP_062063.1), rat transferrin receptor protein 1 (“CD71”, see e.g., UniProtKB Q99376 (SEQ ID NO:11), RefSeq: NP_073203.1), and rat H-2 class II histocompatibility antigen gamma chain (“CD74”, see e.g., UniProtKB P10247 (SEQ ID NO:12), GenBank: CAA32468.1, RefSeq: NP_037201.1 (SEQ ID NO:13)). Rat antigen amino acid sequences are provided in Table 1.

TABLE 1
Rat Antigens

| | |
|---|---|
| RT1A (Rattus | MEAMAPRTLLLLLAAALAPTQTRAGSHSLR |
| norvegicus- | YFYTAVSRPGLGEPRFIAVGYVDDTEFVRF |
| UniProt | DSDAENPRMEPRARWMEREGPEYWEQQTRI |
| P16391- | AKEWEQIYRVDLRTLRGYYNQSEGGSHTIQ |
| GenBank | EMYGCDVGSDGSLLRGYRQDAYDGRDYIAL |
| AAB49324.1) | NEDLKTWTAADFAAQITRNKWERARYAERL |
| SEQ ID NO: 7 | RAYLEGTCVEWLSRYLELGKETLLRSDPPE |
| | AHVTLHPRPEGDVTLRCWALGFYPADITLT |
| | WQLNGEDLTQDMELVETRPAGDGTFQKWAS |
| | VVVPLGKEQNYTCRVEHEGLPKPLSQRWEP |
| | SPSTDSNMETTVIYVILGAVAMIGAVAIIG |
| | AMVAVVRRRKRNTGGKGGDYAPAPGRDSSQ |
| | SSDVSLPDCKA |
| CD26 (Rattus | MKTPWKVLLGLLGVAALVTIITVPVVLLNK |
| norvegicus- | DEAAADSRRTYTLADYLKNTFRVKSYSLRW |
| UniProt | VSDSEYLYKQENNILLFNAEHGNSSIFLEN |
| P14740) | STFEIFGDSISDYSVSPDRLFVLLEYNYVK |
| SEQ ID NO: 8 | QWRHSYTASYSIYDLNKRQLITEEKIPNNT |
| | QWITWSQEGHKLAYVWKNDIYVKIEPHLPS |
| | HRITSTGKENVIFNGINDWVYEEEIFGAYS |
| | ALWWSPNGTFLAYAQFNDTGVPLIEYSFYS |
| | DESLQYPKTVWIPYPKAGAVNPTVKFFIVN |
| | TDSLSSTTTTIPMQITAPASVTTGDHYLCD |
| | VAWVSEDRISLQWLRRIQNYSVMAICDYDK |
| | TTLVWNCPTTQEHIETSATGWCGRFRPAEP |
| | HFTSDGSSFYKIVSDKDGYKHICQFQKDRK |
| | PEQVCTFITKGAWEVISIEALTSDYLYYIS |
| | NEYKEMPGGRNLYKIQLTDHTNKKCLSCDL |
| | NPERCQYYSVSLSKEAKYYQLGCRGPGLPL |
| | YTLHRSTDQKELRVLEDNSALDKMLQDVQM |
| | PSKKLDFIVLNETRFWYQMILPPHFDKSKK |
| | YPLLIDVYAGPCSQKADAAFRLNWATYLAS |
| | TENIIVASFDGRGSGYQGDKIMHAINKRLG |
| | TLEVEDQIEAARQFLKMGFVDSKRVAIWGW |
| | SYGGYVTSMVLGSGSGVFKCGIAVAPVSRW |
| | EYYDSVYTERYMGLPTPEDNLDHYRNSTVM |
| | SRAENFKQVEYLLIHGTADDNVHFQQSAQI |
| | SKALVDAGVDFQAMWYTDEDHGIASSTAHQ |
| | HIYSHMSHFLQQCFSLR |
| CD26 (Rattus | MKTPWKVLLGLLGVAALVTIITVPVVLLNK |
| norvegicus- | DEAAADSARTYTLADYLKNTFRVKSYSLRW |
| GenBank | VSDSEYLYKQENNILLFNAEHGNSSIFLEN |
| AAA41096.1- | STFEIFGDSISDYSVSPDRLFVLLEYNYVK |
| RefSeq | QWRHSYTASYSIYDLNKRQLITEEKIPNNT |
| NP_ | QWITWSQEGHKLAYVWKNDIYVKIEPHLPS |
| 036921.1) | HRITSTGKENVIFNGINDWVYEEEIFGAYS |
| SEQ ID NO: 9 | ALWWSPNGTFLAYAQFNDTGVPLIEYSFYS |
| | DESLQYPKTVWIPYPKAGAVNPTVKFFIVN |
| | TDSLSSTTTTIPMQITAPASVTTGDHYLCD |
| | VAWVSEDRISLQWLRRIQNYSVMAICDYDK |
| | TTLVWNCPTTQEHIETSATGWCGRFRPAEP |
| | HFTSDGSSFYKIVSDKDGYKHICQFQKDRK |
| | PEQVCTFITKGAWEVISIEALTSDYLYYIS |
| | NEYKEMPGGRNLYKIQLTDHTNKKCLSCDL |
| | NPERCQYYSVSLSKEAKYYQLGCRGPGLPL |
| | YTLHRSTDQKELRVLEDNSALDKMLQDVQM |
| | PSKKLDFIVLNETRFWYQMILPPHFDKSKK |
| | YPLLIDVYAGPCSQKADAAFRLNWATYLAS |
| | TENIIVASFDGRGSGYQGDKIMHAINKRLG |
| | TLEVEDQIEAARQFLKMGFVDSKRVAIWGW |
| | SYGGYVTSMVLGSGSGVFKCGIAVAPVSRW |
| | EYYDSVYTERYMGLPTPEDNLDHYRNSTVM |
| | SRAENFKQVEYLLIHGTADDNVHFQQSAQI |
| | SKALVDAGVDFQAMWYTDEDHGIASSTAHQ |
| | HIYSHMSHFLQQCFSLR |
| CD46 (Rattus | MTAAPLTPDPTHPRRRRKSYTFFSLGIYAE |
| norvegicus- | ALLFLLSSLSDACEPPPPFEAMELKDKPKP |
| UniProt | HYAIGEIIEYTCKKGYLYLSPYPMTAICQP |
| Q9ZOM4- | NHTWVPISDHGCIKVQCTMLQDPSFGKVHY |
| GenBank | IDGRFSWGARVKYTCMNGYYMVGMSVLQCE |
| BAA34811.1- | LNGNGDAFWNGHPPSCKKVYCLPPPKIKNG |
| RefSeq | THTFTDIKVFKYHEAVIYSCDPNPGPDKFS |
| NP_062063.1) | LVGPSMLFCAGHNTWSSDPPECKVVKCPFP |
| SEQ ID NO: 10 | VLQNGRQISRTEKKFSYQALVLFQCLEGFY |
| | MEGSSMVVCGAKSSWEPSIPQCLKGPKPHS |
| | TKPPVYSESGYPSPREGIFGQEFDAWIIAL |
| | IVVTSVVGVIVICLIILRCSEYRKK |
| CD71 (Rattus | MMDQARSAFSNLFGGEPLSYTRFSLARQVD |
| norvegicus- | GDNSHVEMKLAADEEENADSNMKASVRKPK |
| UniProt | RFNGRLCFATIAVVIFFLIGFMIGYLGYCK |
| Q99376- | RVEQKEECVRLAEAEEADKSENDETEYVPK |
| GenBank | SSRLFWADLKTLLSEKLNSIEFTDIIKQLS |
| EDM11405.1- | QNTYTPREAGSQKDENLAYYIENLFHDFKF |
| RefSeq | SKVWRDEHYVKIQVKNSVSQNLVTINSGSN |
| NP_073203.1) | IDPVEAPEGYVAFSKAGEVTGKLVHANFGT |
| SEQ ID NO: 11 | KKDFEELNYSVNGSLVIVRAGKITFAEKVA |
| | NAQSFNAIGVLIYMDRNTFPVVEADLQFFG |
| | HAHLGTGDPYTPGFPSFNHTQFPPSQSSGL |
| | PSIPVQTISRAAAEKLFKNMEGNCPPSWNI |
| | DSSCKLELSQNQNVKLTVNNVLKETRILNI |
| | FGVIKGYEEPDRYIVVGAQRDAWGPGVAKS |
| | SVGTGLLLKLAQVFSDMISKDGFRPSRSII |
| | FASWTAGDYGAVGATEWLEGYLSSLHLKAF |
| | TYINLDKVVLGTSNFKVSASPLLYTLMGKI |
| | MQDVKHPIDGKYLYRDSNWISKIEELSLDN |
| | AAFPFLAYSGIPAVSFCFCEDEDYPYLGTK |
| | LDTYEILIQKVPQLNQMVRTAAEVAGQFII |
| | KLTHDIELTLDYEMYNSKLLSFMKDLNQFK |
| | ADIKDMGLSLQWLYSARGDYFRATSRLTTD |
| | FHNAEKTNRFVMREINDRIMKVEYHFLSPY |
| | VSPRESPFRHIFWGSGSHTLSALVENLRLR |
| | QKNITAFNETLFRNQLALATWTIQGVANAL |
| | SGDIWNIDNEF |
| CD74 (Rattus | MDDQRDLISNHEQLPILGQRARAPESNCNR |
| norvegicus- | GVLYTSVSVLVALLLAGQATTAYFLYQQQG |
| UniProt | RLDKLTVTSQNLQLENLRMKLPKSAKPVSP |
| P10247- | MRMATPLLMRPLSMDNMLQAPVKNVTKYGN |
| GenBank | MTQDHVMHLLTKSGPVNYPQLKGSFPENLK |
| CAA32468.1) | HLKNSMNGLDWKVFESWMKQWLLFEMSKNS |
| SEQ ID NO: 12 | LEEKQPTQTPPKVLTKCQEEVSHIPDVHPG |
| | AFRPKCDENGNYMPLQCHGSTGYCWCVFPN |
| | GTEVPHTKSRGRHNCSEPLDMEDPSSGLGV |
| | TKQDMGQMFL |
| CD74 (Rattus | MDDQRDLISNHEQLPILGQRARAPESNCNR |
| norvegicus- | GVLYTSVSVLVALLLAGQATTAYFLYQQQG |
| RefSeq | RLDKLTVTSQNLQLENLRMKLPKSAKPVSP |
| NP_037201.1) | MRMATPLLMRPLSMDNMLQAPVKNVTKYGN |
| SEQ ID NO: 13 | MTQDHVMHLLTKSGPVNYPQLKGSFPENLK |
| | HLKNSMNGLDWKVFESWMKQWLLFEMSKNS |
| | LEEKQPTQTPPKEPLDMEDPSSGLGVTKQD |
| | MGQMFL |

As can be seen in FIG. 1, the antibody directed to RT1A bound a substantially higher percentage of rat cells as compared to the percent of total rat cells bound by antibodies to the other candidate target antigens, CD26, CD46, CD71, and CD74. Accordingly, in this example, rat RT1A was chosen as the target pan-rat antigen for further antibody screening and evaluation in negative selection method development.

Different anti-RT1A monoclonal antibodies were evaluated for binding to heterogeneous populations of rat cells. The different clones evaluated bound widely varied percentages of the total rat cells present in the population. For example, as shown in FIG. 2, three different anti-RT1A monoclonal antibodies, IgG1 MRC clone OX-18 (see e.g., Fukumoto, T. et al. (1982) Eur J Immunol. 12 (3): 237-43; herein “OX-18”), IgG2a MRC clone OX-27 (see e.g., Jefferies et al. (1985) J Exp Med. 162(1):117-27; herein “OX-27”), and IgG1 clone F16-4-4 (see e.g., Hart & Fabre (1981) Transplantation. 31(5):318-325; herein “F-16”) were independently incubated with aliquots of a heterogeneous population of rat liver cells and the percentage of cells in the population bound by each clone was evaluated by cell count and measuring the proportion between the retained and the total number of cells between the retained and flow through fractions. The results showed that while OX-18 and F-16 bound 60% or greater of the total cells in the population, OX-27 bound less than 10%.

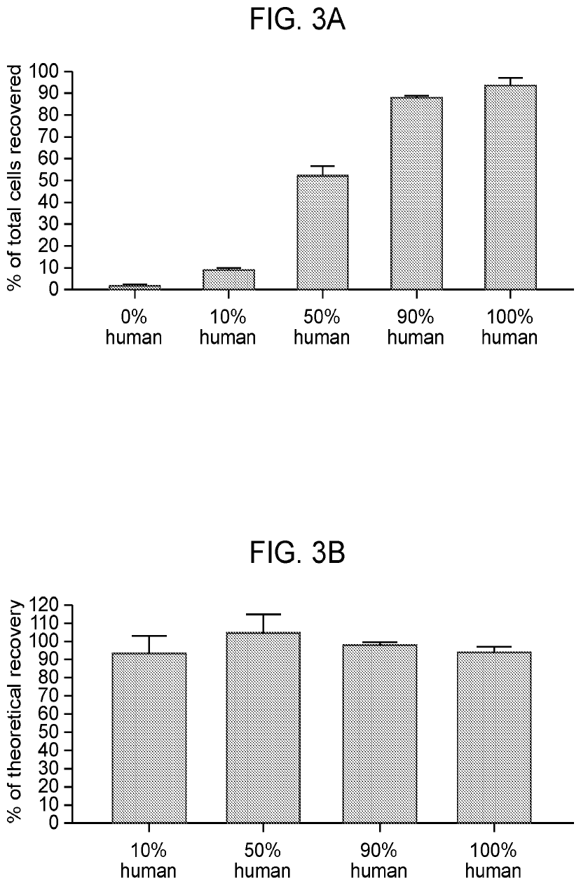

F-16 was chosen for further characterization of the purification of human hepatocytes from xenomixtures containing human and rat cells using magnetic anti-RT1A-based negative selection. For example, as shown in FIG. 3A, the recovery of human cells through such negative selection was evaluated using defined xenomixtures containing various ratios of rat to human cells (100% rat cells (i.e., 0% Human), 10% human (i.e., 90% rat), 50% human (i.e., 50% rat), 90% human (i.e., 10% rat), and 100% human (i.e., 0% rat)). Briefly, each xenomixture containing the indicated percentages of human hepatocytes and rat cells was incubated with F-16 primary antibody and a magnetic-bead-bound secondary antibody. Then the antibody-containing xenomixture was flowed through a column with a magnetic force applied to the column. The flow-through was then evaluated by cell count and measuring the proportion between the retained and the total number of cells between the retained and flow-through fractions for the desired human hepatocytes present.

As shown, all or nearly all of the human cells present in the xenomixture were readily retrieved using the anti-RT1A-based negative selection approach. FIG. 3B re-displays the data as percent of theoretical recovery from the human-cell-containing xenomixtures and shows that all or nearly all human hepatocytes that could theoretically be recovered were recovered despite the wide range in starting ratios of human cells to non-human cells.

Collectively, these data demonstrated the potential for magnetic anti-pan-non-human antibody-based negative selection as an effective method for the purification of human hepatocytes from xenomixtures. Moreover, these data demonstrate the selection of useful non-human antibodies, the binding of a wide range of undesired non-human cells, e.g., rat cells, by a selected pan-non-human, e.g., pan-rat, antibody, and the effective collection of human hepatocytes following negative selection of the undesired cells.

For further method development, as described below, an anti-RT1A antibody was produced, having the heavy and light chain coding sequences encoded by the mammalian codon optimized sequences of SEQ ID NO:14 and SEQ ID NO:17, respectively. Nucleotide and amino acid sequences for the anti-RT1A antibody are provided in Table 2. “aRT1”, anti-RT1A antibody; “H”, heavy chain; “LC”, light chain; “CDR”, complementarity determining region.

TABLE 2
anti-RT1A antibody nucleotide and
amino acid sequences

| | |
|---|---|
| aRT1 | GAGGTGCAGCTTCAGGAGTCAGGAC |
| codon optimized | CTAGCCTCGTGAAACCTTCTCAGAC |
| heavy chain- | TCTACAGTGGTAGCACTTACTACAA |
| encoding | TCCATCTCTCAAAAGTCGAATCTCC |
| nucleotide | ATCACTGTCCCTCACCTGTTCTGTC |
| sequence | ACTGGCGACTCCATCACCAGTGGTT |
| SEQ ID NO: 14 | ACTGGAATCGAGACACATCCAAGAA |
| | CCAGTACTACCTGCAGTTGAATTCT |
| | GTGACTACTGCTGGATCCGGAAATT |
| | CCCAGGGAATAAACTTGAGTACATG |
| | GGGTACATAAGCAGGACACAGCCAC |
| | ATATTACTGTGCAAGTCATAGCCAC |
| | TGGTACTTCGATGTCTCTGTCTATC |
| | CACTGGCCCCTGGATCTGCTGCCCA |
| | AACTAACTCCATGGTGATGGGGCGC |
| | AGGGACCACGGTCACCGTCTCCTCA |
| | GCCAAAACGACACCCCCACCCTGGG |
| | ATGCCTGGTCAAGGGCTATTTCCCT |
| | GAGCCAGTGACAGTGACCTGGAACT |
| | CTGGATCCCTGTCCAGCGGTGTGCA |
| | CACCTTCCCAGCTGTCCTGCAGTCT |
| | GACCTCTACACTCTGAGCAGCTCAG |
| | TGACTGTCCCCTCCAGCACCTGGCC |
| | CAGCGAGACCGTCACCTGCAACGTT |
| | GCCCACCCGGCCAGCAGCACCAAGG |
| | TGGACAAGAAAATTGTGCCCAGGGA |
| | TTGTGGTTGTAAGCCTTGCATATGT |
| | ACAGTCCCAGAAGTATCATCTGTCT |
| | TCATCTTCCCCCCAAAGCCCAAGGA |
| | TGTGCTCACCATTACTCTGACTCCT |
| | AAGGTCACGTGTGTTGTGGTAGACA |
| | TCAGCAAGGATGATCCCGAGGTCCA |
| | GTTCAGCTGGTTTGTAGATGATGTG |
| | GAGGTGCACACAGCTCAGACGCAAC |
| | CCCGGGAGGAGCAGTTCAACAGCAC |
| | TTTCCGCTCAGTCAGTGAACTTCCC |
| | ATCATGCACCAGGACTGGCTCAATG |
| | GCAAGGAGTTCAAATGCAGGGTCAA |
| | CAGTGCAGCTTTCCCTGCCCCCATC |
| | GAGAAAACCATCTCCAAAACCAAAG |
| | GCAGACCGAAGGCTCCACAGGTGTA |
| | CACCATTCCACCTCCCAAGGAGCAG |
| | ATGGCCAAGGATAAAGTCAGTCTGA |
| | CCTGCATGATAACAGACTTCTTCCC |
| | TGAAGACATTACTGTGGAGTGGCAG |
| | TGGAATGGGCAGCCAGCGGAGAACT |
| | ACAAGAACACTCAGCCCATCATGGA |
| | CACAGATGGCTCTTACTTCGTCTAC |
| | AGCAAGCTCAATGTGCAGAAGAGCA |
| | ACTGGGAGGCAGGAAATACTTTCAC |
| | CTGCTCTGTGTTACATGAGGGCCTG |
| | CACAACCACCATACTGAGAAGAGCC |
| | TCTCCCACTCTCCTGGTAAATGA |
| aRT1-HC (full | EVQLQESGPSLVKPSQTLSLTCSVT |
| length-signal | GDSITSGYWNWIRKFPGNKLEYMGY |
| peptide not | ISYSGSTYYNPSLKSRISITRDTSK |
| shown) | NQYYLQLNSVTTEDTATYYCASHSH |
| mouse IgG1 | WYFDVWGAGTTVTVSSAKTTPPSVY |
| isotype | PLAPGSAAQTNSMVTLGCLVKGYFP |
| SEQ ID NO: 15 | EPVTVTWNSGSLSSGVHTFPAVLQS |
| | DLYTLSSSVTVPSSTWPSETVTCNV |
| | AHPASSTKVDKKIVPRDCGCKPCIC |
| | TVPEVSSVFIFPPKPKDVLTITLTP |
| | KVTCVVVDISKDDPEVQFSWFVDDV |
| | EVHTAQTQPREEQFNSTFRSVSELP |
| | IMHQDWLNGKEFKCRVNSAAFPAPI |
| | EKTISKTKGRPKAPQVYTIPPPKEQ |
| | MAKDKVSLTCMITDFFPEDITVEWQ |
| | WNGQPAENYKNTQPIMDTDGSYFVY |
| | SKLNVQKSNWEAGNTFTCSVLHEGL |
| | HNHHTEKSLSHSPGK |
| aRT1-HC | EVQLQESGPSLVKPSQTLSLTCSVT |
| (variable | GDSITSGYWNWIRKFPGNKLEYMGY |
| region) | ISYSGSTYYNPSLKSRISITRDTSK |
| SEQ ID NO: 16 | NQYYLQLNSVTTEDTATYYCASHSH |
| | WYFDVWGAGTTVTVSS |
| aRT1-HC-CDR1 | GDSITSGY |
| (IMGT) | |
| SEQ ID NO: 1 | |
| aRT1-HC-CDR2 | ISYSGST |
| (IMGT) | |
| SEQ ID NO: 2 | |
| aRT1-HC-CDR3 | ASHSHWYFDV |
| (IMGT) | |
| SEQ ID NO: 3 | |
| aRT1 | GATATCCAGATGACACAGACTACAT |
| codon optimized | CCTCCCTGTCTGCCTCTCTGGGAGA |
| light chain- | CAGAGTCACCATCAGTTGCAGGGCA |
| encoding | AGTCAGGACATTAGCAATTATTTAA |
| nucleotide | ACTGGTATCAGCAGAAACCAGATGG |
| sequence | AACTGTTAAACTCCTGATCTACTAC |
| SEQ ID NO: 17 | ACATCAAGATTACACTCAGGAGTCC |
| | CATCAAGGTTCAGTGGCAGTGGGTC |
| | TGGAACAGATTATTCTCTCACCATT |
| | AGCAACCTGGAGCAAGAAGATATTG |
| | CCACTTACTTTTGCCAACAGGGTAA |
| | TACGCTTCCGTGGACGTTCGGTGGA |
| | GGCACCAAGCTGGAAATCAAACGGG |
| | CTGATGCTGCACCAACTGTATCCAT |
| | CTTCCCACCATCCAGTGAGCAGTTA |
| | ACATCTGGAGGTGCCTCAGTCGTGT |
| | GCTTCTTGAACAACTTCTACCCCAA |
| | AGACATCAATGTCAAGTGGAAGATT |
| | GATGGCAGTGAACGACAAAATGGCG |
| | TCCTGAACAGTTGGACTGATCAGGA |
| | CAGCAAAGACAGCACCTACAGCATG |
| | AGCAGCACCCTCACGTTGACCAAGG |
| | ACGAGTATGAACGACATAACAGCTA |
| | TACCTGTGAGGCCACTCACAAGACA |
| | TCAACTTCACCCATTGTCAAGAGCT |
| | TCAACAGGAATGAGTGTTAG |
| aRT1-LC (full | DIQMTQTTSSLSASLGDRVTISCRA |
| length-signal | SQDISNYLNWYQQKPDGTVKLLIYY |
| peptide not | TSRLHSGVPSRFSGSGSGTDYSLTI |
| shown) | SNLEQEDIATYFCQQGNTLPWTFGG |
| mouse kappa | GTKLEIKRADAAPTVSIFPPSSEQL |
| isotype | TSGGASVVCFLNNFYPKDINVKWKI |
| SEQ ID NO: 18 | DGSERQNGVLNSWTDQDSKDSTYSM |
| | SSTLTLTKDEYERHNSYTCEATHKT |
| | STSPIVKSFNRNEC |
| aRT1-LC | DIQMTQTTSSLSASLGDRVTISCRA |
| (variable | SQDISNYLNWYQQKPDGTVKLLIYY |
| region) | TSRLHSGVPSRFSGSGSGTDYSLTI |
| SEQ ID NO: 19 | SNLEQEDIATYFCQQGNTLPWTFGG |
| | GTKLEIK |
| aRT1-LC-CDR1 | QDISNY |
| (IMGT) | |
| SEQ ID NO: 4 | |
| aRT1-LC-CDR2 | YTS |
| (IMGT) | |
| SEQ ID NO: 5 | |
| aRT1-LC-CDR3 | QQGNTLPWT |
| (IMGT) | |
| SEQ ID NO: 6 | |
| aRT1-HC-FR1_ | EVQLQESGPSLVKPSQTLSLTCSVT |
| Chothia | |
| SEQ ID NO: 20 | |
| aRT1-HC-FR1_ | EVQLQESGPSLVKPSQTLSLTCSVT |
| AbM | |
| SEQ ID NO: 21 | |
| aRT1-HC-FR1_ | EVQLQESGPSLVKPSQTLSLTCSVT |
| Kabat | GDSIT |
| SEQ ID NO: 22 | |
| aRT1-HC-FR1_ | EVQLQESGPSLVKPSQTLSLTCSVT |
| Contact | GDSI |
| SEQ ID NO: 23 | |
| aRT1-HC-FR1_ | EVQLQESGPSLVKPSQTLSLTCSVT |
| IMGT | |
| SEQ ID NO: 24 | |
| aRT1-HC-CDR1_ | GDSITSG |
| Chothia | |
| SEQ ID NO: 25 | |
| aRT1-HC-CDR1_ | GDSITSGYWN |
| AbM | |
| SEQ ID NO: 26 | |
| aRT1-HC-CDR1_ | SGYWN |
| Kabat | |
| SEQ ID NO: 27 | |
| aRT1-HC-CDR1_ | TSGYWN |
| Contact | |
| SEQ ID NO: 28 | |
| aRT1-HC-FR2_ | YWNWIRKFPGNKLEYMGYI |
| Chothia | |
| SEQ ID NO: 29 | |
| aRT1-HC-FR2_ | WIRKFPGNKLEYMG |
| AbM | |
| SEQ ID NO: 30 | |
| aRT1-HC-FR2_ | WIRKFPGNKLEYMG |
| Kabat | |
| SEQ ID NO: 31 | |
| aRT1-HC-FR2_ | WIRKFPGNKLE |
| Contact | |
| SEQ ID NO: 32 | |
| aRT1-HC-FR2_ | WNWIRKFPGNKLEYMGY |
| IMGT | |
| SEQ ID NO: 33 | |
| aRT1-HC-CDR2_ | SYSGS |
| Chothia | |
| SEQ ID NO: 34 | |
| aRT1-HC-CDR2_ | YISYSGSTY |
| AbM | |
| SEQ ID NO: 35 | |
| aRT1-HC-CDR2_ | YISYSGSTYYNPSLKS |
| Kabat | |
| SEQ ID NO: 36 | |
| aRT1-HC-CDR2_ | YMGYISYSGSTY |
| Contact | |
| SEQ ID NO: 37 | |
| aRT1-HC-FR3_ | TYYNPSLKSRISITRDTSKNQYYLQ |
| Chothia | LNSVTTEDTATYYCAS |
| SEQ ID NO: 38 | |
| aRT1-HC-FR3_ | YNPSLKSRISITRDTSKNQYYLQLN |
| AbM | SVTTEDTATYYCAS |
| SEQ ID NO: 39 | |
| aRT1-HC-FR3_ | RISITRDTSKNQYYLQLNSVTTEDT |
| Kabat | ATYYCAS |
| SEQ ID NO: 40 | |
| aRT1-HC-FR3_ | YNPSLKSRISITRDTSKNQYYLQLN |
| Contact | SVTTEDTATYYC |
| SEQ ID NO: 41 | |
| aRT1-HC-FR3_ | YYNPSLKSRISITRDTSKNQYYLQL |
| IMGT | NSVTTEDTATYYC |
| SEQ ID NO: 42 | |
| aRT1-HC-CDR3_ | HSHWYFDV |
| Chothia | |
| SEQ ID NO: 43 | |
| aRT1-HC-CDR3_ | HSHWYFDV |
| AbM | |
| SEQ ID NO: 44 | |
| aRT1-HC-CDR3_ | HSHWYFDV |
| Kabat | |
| SEQ ID NO: 45 | |
| aRT1-HC-CDR3_ | ASHSHWYFD |
| Contact | |
| SEQ ID NO: 46 | |
| aRT1-HC-FR4_ | WGAGTTVTVSS |
| Chothia | |
| SEQ ID NO: 47 | |
| aRT1-HC-FR4_ | WGAGTTVTVSS |
| AbM | |
| SEQ ID NO: 48 | |
| aRT1-HC-FR4_ | WGAGTTVTVSS |
| Kabat | |
| SEQ ID NO: 49 | |
| aRT1-HC-FR4_ | VWGAGTTVTVSS |
| Contact | |
| SEQ ID NO: 50 | |
| aRT1-HC-FR4_ | WGAGTTVTVSS |
| IMGT | |
| SEQ ID NO: 51 | |
| aRT1-LC-FR1_ | DIQMTQTTSSLSASLGDRVTISC |
| Chothia | |
| SEQ ID NO: 52 | |
| aRT1-LC-FR1_ | DIQMTQTTSSLSASLGDRVTISC |
| AbM | |
| SEQ ID NO: 53 | |
| aRT1-LC-FR1_ | DIQMTQTTSSLSASLGDRVTISC |
| Kabat | |
| SEQ ID NO: 54 | |
| aRT1-LC-FR1_ | DIQMTQTTSSLSASLGDRVTISCRA |
| Contact | SQDI |
| SEQ ID NO: 55 | |
| aRT1-LC-FR1_ | DIQMTQTTSSLSASLGDRVTISCRA |
| IMGT | S |
| SEQ ID NO: 56 | |
| aRT1-LC-CDR1_ | RASQDISNYLN |
| Chothia | |
| SEQ ID NO: 57 | |
| aRT1-LC-CDR1_ | RASQDISNYLN |
| AbM | |
| SEQ ID NO: 58 | |
| aRT1-LC-CDR1_ | RASQDISNYLN |
| Kabat | |
| SEQ ID NO: 59 | |
| aRT1-LC-CDR1_ | SNYLNWY |
| Contact | |
| SEQ ID NO: 60 | |
| aRT1-LC-FR2_ | WYQQKPDGTVKLLIY |
| Chothia | |
| SEQ ID NO: 61 | |
| aRT1-LC-FR2_ | WYQQKPDGTVKLLIY |
| AbM | |
| SEQ ID NO: 62 | |
| aRT1-LC-FR2_ | WYQQKPDGTVKLLIY |
| Kabat | |
| SEQ ID NO: 63 | |
| aRT1-LC-FR2_ | QQKPDGTVK |
| Contact | |
| SEQ ID NO: 64 | |
| aRT1-LC-FR2_ | LNWYQQKPDGTVKLLIY |
| IMGT | |
| SEQ ID NO: 65 | |
| aRT1-LC-CDR2_ | YTSRLHS |
| Chothia | |
| SEQ ID NO: 66 | |
| aRT1-LC-CDR2_ | YTSRLHS |
| AbM | |
| SEQ ID NO: 67 | |
| aRT1-LC-CDR2_ | YTSRLHS |
| Kabat | |
| SEQ ID NO: 68 | |
| aRT1-LC-CDR2_ | LLIYYTSRLH |
| Contact | |
| SEQ ID NO: 69 | |
| aRT1-LC-FR3_ | GVPSRFSGSGSGTDYSLTISNLEQE |
| Chothia | DIATYFC |
| SEQ ID NO: 70 | |
| aRT1-LC-FR3_ | GVPSRFSGSGSGTDYSLTISNLEQE |
| AbM | DIATYFC |
| SEQ ID NO: 71 | |
| aRT1-LC-FR3_ | GVPSRFSGSGSGTDYSLTISNLEQE |
| Kabat | DIATYFC |
| SEQ ID NO: 72 | |
| aRT1-LC-FR3_ | SGVPSRFSGSGSGTDYSLTISNLEQ |
| Contact | EDIATYFC |
| SEQ ID NO: 73 | |
| aRT1-LC-FR3_ | SRLHSGVPSRFSGSGSGTDYSLTIS |
| IMGT | NLEQEDIATYFC |
| SEQ ID NO: 74 | |
| aRT1-LC-CDR3_ | QQGNTLPWT |
| Chothia | |
| SEQ ID NO: 75 | |
| aRT1-LC-CDR3_ | QQGNTLPWT |
| AbM | |
| SEQ ID NO: 76 | |
| aRT1-LC-CDR3_ | QQGNTLPWT |
| Kabat | |
| SEQ ID NO: 77 | |
| aRT1-LC-CDR3_ | QQGNTLPW |
| Contact | |
| SEQ ID NO: 78 | |
| aRT1-LC-FR4_ | FGGGTKLEIKRA |
| Chothia | |
| SEQ ID NO: 79 | |
| aRT1-LC-FR4_ | FGGGTKLEIKRA |
| AbM | |
| SEQ ID NO: 80 | |
| aRT1-LC-FR4_ | FGGGTKLEIKRA |
| Kabat | |
| SEQ ID NO: 81 | |
| aRT1-LC-FR4_ | TFGGGTKLEIKRA |
| Contact | |
| SEQ ID NO: 82 | |
| aRT1-LC-FR4_ | FGGGTKLEIKRA |
| IMGT | |
| SEQ ID NO: 83 | |

### Example 10—Collection and Enrichment of Hepatocyte Populations from In Vivo Bioreactors

In this example, human hepatocytes were collected from the humanized liver tissue of in vivo bioreactors, such as e.g., Fah−/−, IL2rg−/−, and Rag1−/− or Rag2−/− rats, following transplantation, engraftment, and expansion of human hepatocytes in the bioreactors. Useful solutions, including e.g., perfusion buffer 1 (PB1), perfusion buffer2 (PB2), collagenase MA solution, BP protease solution, complete hepatocyte plating medium, and buffered saline, were prepared or retrieved in advance.

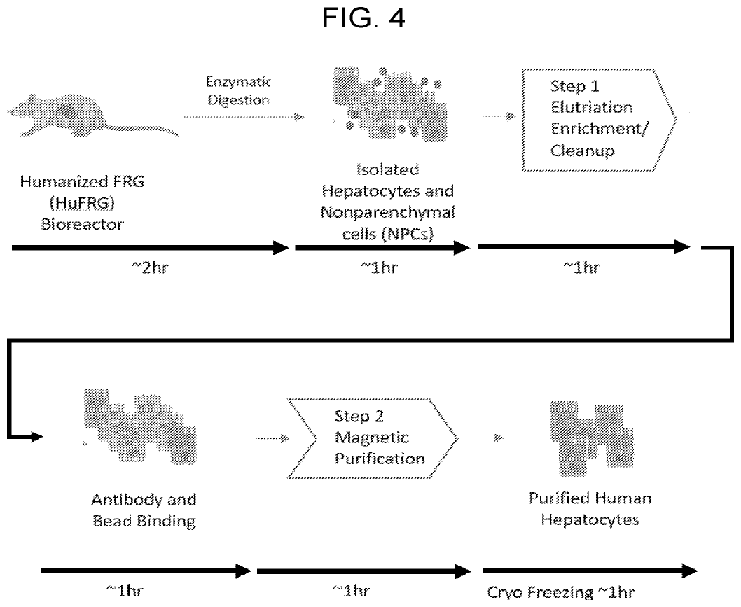

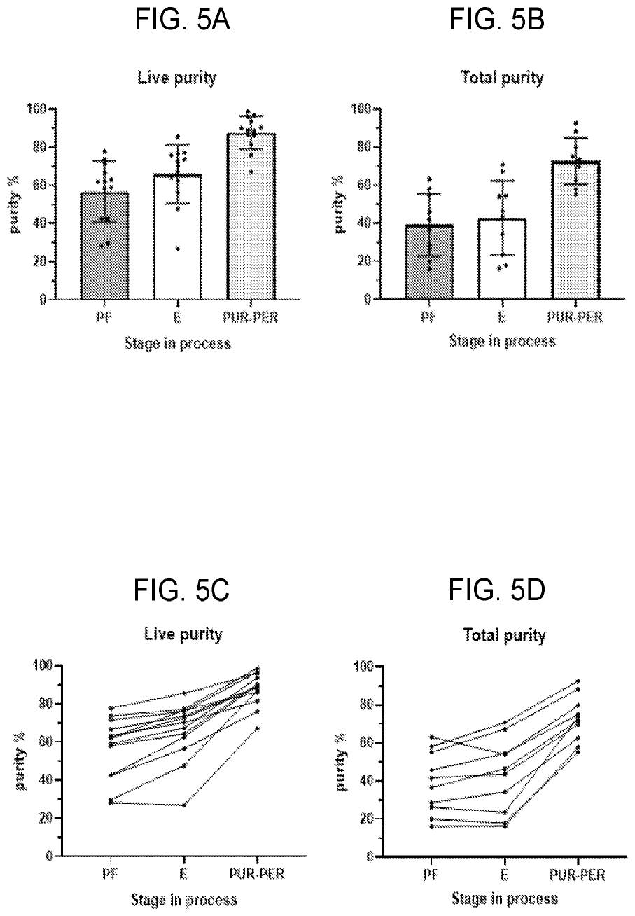

For simplicity, the perfusion of a single rat bioreactor will be described, however, this example will be understood to also describe the perfusion of multiple rat bioreactor livers at a time by performing the procedure on multiple livers sequentially, simultaneously (i.e., in parallel), or some combination thereof. A perfusion apparatus, including a perfusion pump system connected by sterile pump tubing to containers containing P1 and P2 buffers, was prepared in advance for each liver processed in parallel. A non-limiting example overview of the isolation and enrichment workflow for processing of about 10 billion human hepatocytes is provided in FIG. 4, demonstrating that, in some embodiments, the entire process from obtaining humanized liver to cryopreservation of purified human hepatocytes may be completed in about 7 hours or less.

As shown in FIG. 4, hepatocytes, along with other cell types, were perfused from a humanized FRG rat (huFRG) bioreactor. The bioreactor, containing a humanized liver, was fully anesthetized and the animal's abdomen was sterilized and surgically opened to access the liver and surrounding vessels, including the portal vein (PV) and inferior vena cava (IVC). The PV was cannulated less than 1 inch from the liver using a cannula needle and the cannula was secured. The secured cannula was attached to the P1 buffer perfusion line and P1 buffer was flowed into the liver via the PV. The IVC was punctured using a cannula needle and P1 buffer was allowed to drain from the liver. Pressure and flow rate were constantly monitored and adjusted as necessary. P1 buffer was continually flowed through the liver until all blood was drained.

Next, pump settings were adjusted to switch from P1 buffer to P2 buffer, containing collagenase and protease, and the flow rate was reduced. During perfusion with P2 buffer, the liver was monitored for structural degradation and surgically removed from the animal when breakage was detected. The resected liver was placed in a container of cold complete hepatocyte plating medium, Glisson's capsule was removed, and the liver was mechanically dissociated. In this example, the perfusates from multiple bioreactor livers processed in parallel were pooled into an initial container, e.g., a sterile bottle, sterile cell transfer bag, or the like, in preparation for the hepatocyte enrichment procedure (also sometimes referred to as “clean up”, e.g., as in FIG. 4).

The initial container containing the pooled liver perfusates, containing human hepatocytes, bioreactor hepatocytes, and other cell types (cell suspension), was connected to a container of EB, a hepatocyte collection container, and a waste collection container using a sterile tubing set and a tube welder. The connected containers and associated tubing were connected to an elutriator. Using the elutriator, all tubing lines were primed with EB and then a cell bed was formed within the elutriation chamber using the cell suspension. By varying the centrifugal force in the elutriation chamber under a constant flow rate, an elutriation fraction that preferentially contained human hepatocytes was retained, washed and then collected from the elutriator into the sterile collection container. The collected fraction was enriched for human hepatocytes as compared to the initial perfusate. By this method the perfusate xenomixture, containing human and rat cells, was preferentially enriched for human cells and human hepatocytes specifically. Collection was continued until the initial container containing the perfusate was emptied.

By this method undesired cell types, including bioreactor cells and non-parenchymal cells (NPCs), and debris are removed allowing for the collection and rapid enrichment of desired human hepatocytes in a closed, sterile system. In some instances, cell counts and viability measurements are taken to measure enrichment performance.

As described in more detail below, it was unexpectedly discovered that human hepatocytes and rat hepatocytes have differences in size and density characteristics that allow for the separation of human and bioreactor hepatocyte populations by elutriation. However, it was similarly discovered that, due to some overlap in the size and density characteristics of human hepatocytes and rat bioreactor cell types, including rat hepatocytes and other rat cell types, the human cells cannot be entirely separated from the rat bioreactor cells by elutriation alone. Accordingly, the collected elutriation fraction enriched for hepatocytes includes a xenomixture of human hepatocytes and rat bioreactor cells, including rat hepatocytes and other rat cell types.

Negative selection of the non-human cell types from the xenomixture was employed to further enrich for the desired human hepatocytes and remove contaminating bioreactor cells. In this example, the rat-specific monoclonal antibody (herein “anti-RT1A”, the heavy and light chain sequences of which are provided above) that specifically binds to rat RT1A class I histocompatibility antigen (see e.g., SEQ ID NO:7), which is the rat homolog of human HLA class I histocompatibility antigen, was used in a magnetic separation procedure.

In brief, anti-RT1A was co-incubated with the cell xenomixture and a magnetic bead conjugated secondary antibody that specifically binds to anti-RT1A. Following incubation, negative selection was applied by bringing a magnet into proximity with the cell mixture to sequester the antibody-bound non-human cells. The free human hepatocytes were separated from the non-human cells and collected to produce a population further enriched for the expanded human hepatocytes. Optionally, the enriched human hepatocyte population was further purified by density gradient centrifugation, e.g., using percoll or a similar gradient component, to remove debris, non-viable cells, and/or other contaminants where present.

FIG. 5A-FIG. 5H provide examples showing the purification and enrichment achieved at various points in the procedure during non-optimized trial runs. For example, FIG. 5A and FIG. 5B show the purity assessed, using a nucleocounter, and calculated as the percent of either all (FIG. 5B, “total purity”) or all live (FIG. 5A, “live purity”) human hepatocytes present in various runs (each run encompassing multiple liver perfusates) at different points in the processing procedure, such as: following perfusion of humanized bioreactor livers (“PF”), following elutriation (“E”), and following magnetic purification and percoll cleanup (“PUR-PER”). The measurements of “Live Purity” and “Total Purity” are representative of the purity obtained when a clean-up step of nonviable cells is and is not employed, respectively. Bars indicate the mean of all displayed runs.

As can be seen in FIG. 5A and FIG. 5B, each stage of the enrichment and purification procedure increases the hepatocyte purity on average and the procedures employed as a whole greatly increase the overall purity, e.g., resulting in numerous individual preparations containing live human hepatocytes at greater than 80% purity. FIG. 5C follows the live hepatocyte purity of individual runs at points PF, E, and PUR-PER of the procedure, with FIG. 5D following the corresponding total hepatocyte purity.

Further supporting the effectiveness of the enrichment and purification procedures, FIG. 5E through FIG. 5H include measurements showing the purity of individual liver perfusates (i.e., “Pre combining”), which were subsequently pooled and processed through the procedure with the corresponding live hepatocyte purities determined at the PF, E, and PUR-PER stages of the process. These data reveal, not only that the purity of each run is progressively increased on average, but also the progressive increases in purity of each individual perfusate over the process. Further studies indicated, surprisingly, that use of Percoll density gradient centrifugation did not have a significant effect on purity and served only to increase the percent viability of the hepatocyte preparations.

Following sufficient enrichment and purification, expanded human hepatocytes are cryopreserved essentially as described in Example 3.

### Example 11—Comparisons of Density Centrifugation-Based and Elutriation-Based Approaches for Human Hepatocyte Enrichment

During evaluation of elutriation as a centrifugal-sedimentation-free strategy to be used in a process for collecting human hepatocytes from a xenomixture produced from the humanized liver of an in vivo bioreactor, it was unexpectedly discovered that a substantial portion of Fah-deficient bioreactor hepatocytes are significantly larger than, and have a cell density different from, engrafted/expanded human hepatocytes. Accordingly, elutriation was further evaluated as a strategy for the enrichment of human hepatocytes from the xenomixture.

For example, following human hepatocyte expansion in an FRG rat bioreactor, cells were perfused from the humanized rat liver and mechanically filtered to obtain a xenomixture containing human hepatocytes and various rat cell types. This xenomixture is sometimes referred to as the bioreactor “post-filter”. An aliquot of the post-filter was retained for analysis and the remaining post-filter was processed by elutriation, to produce an elutriated sample. An aliquot of the elutriated sample was retained for analysis and then subjected to anti-RT-1A antibody-based magnetic negative selection, as described above, to produce a purified population of human hepatocytes. The retained aliquots and final purified preparation were assessed by flow cytometry to measure the percent human purity at each stage in the process. This analysis revealed that elutriation increased the human cell purity, from the post-filter, by greater than 5%, and the antibody-based magnetic negative selection increased the human cell purity, from the elutriated sample, by greater than 20%.

Correspondingly, it was found from this example that elutriation was effective for increasing the human cell purity and enriching for desired hepatocytes from xenomixtures. Elutriation was further assessed for any impact on the function of the enriched human hepatocytes.

Functional characteristics of human hepatocytes isolated using a Percoll-based process or an elutriation-based process were compared. Specifically, three different lots of hepatocytes, each obtained from a different donor liver processed in-house, were each split into two separate fractions and the corresponding fractions were processed using similar protocols differing only in whether the cells were subjected to Percoll density gradient centrifugation or elutriation. After processing, the isolated hepatocytes were assessed for plateability, attachment efficiency, ammonia detoxification, human albumin production, A1AT production, and CYP3A4 activity and the performance of the Percoll-processed and elutriated fractions were compared.

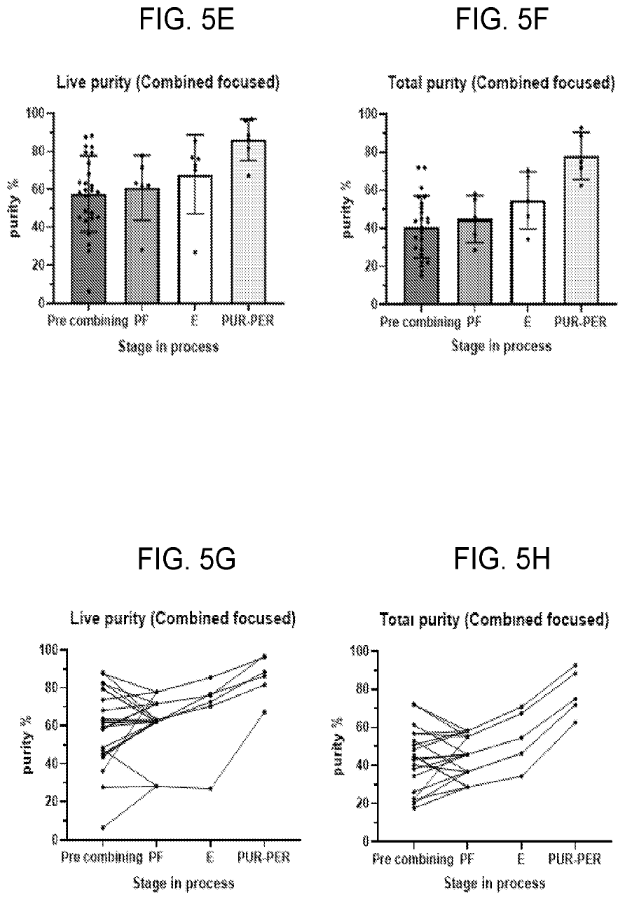

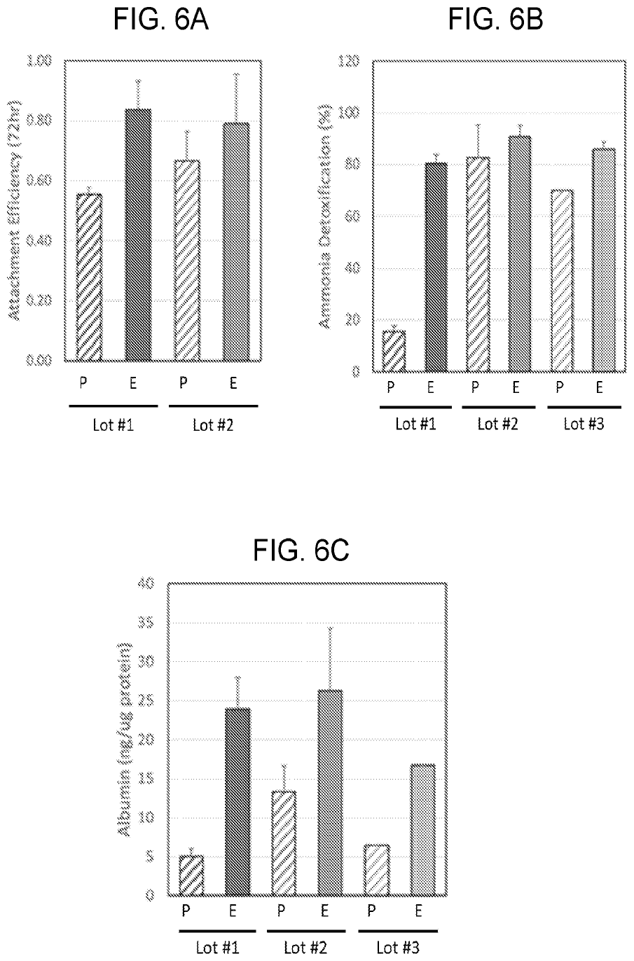

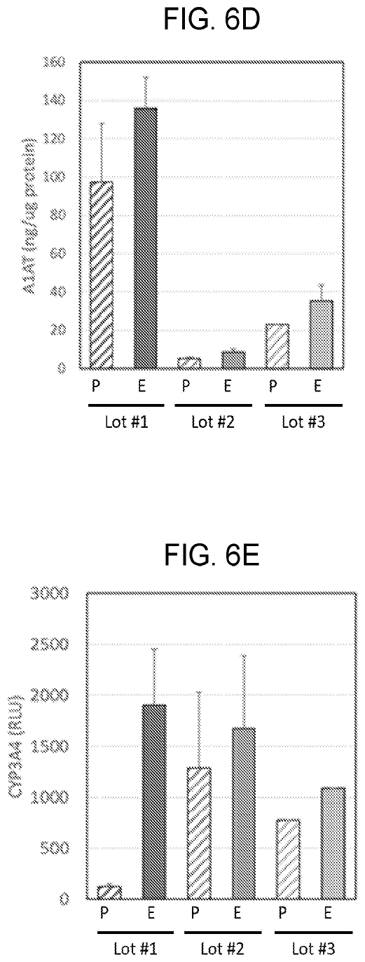

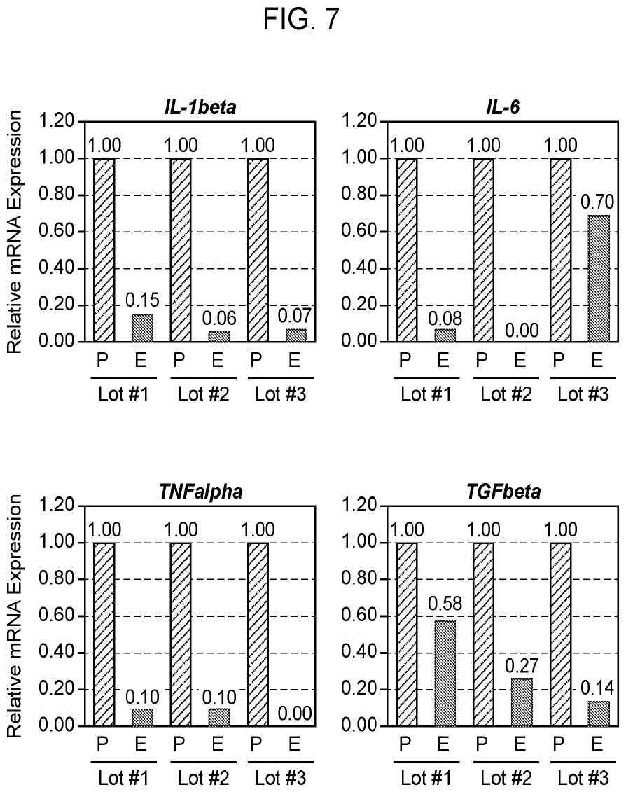

FIG. 6A-6E provide the results of the attachment efficiency (FIG. 6A), ammonia detoxification (FIG. 6B), human albumin production (FIG. 6C), alpha-1 antitrypsin (A1AT) production (FIG. 6D), and cytochrome P450 3A4 (CYP3A4) activity (FIG. 6E) assays for Lots 1, 2, and 3 processed using Percoll (“P”) or elutriation (“E”). Data is not shown for the plateability, which demonstrated enhanced plateability in elutriated fractions of lots 1 and 2 as compared to corresponding Percoll-processed fractions of lots 1 and 2. Note that plateability and attachment results are not available for lot 3.

Collectively, these results surprisingly demonstrated that, in each assay performed, the hepatocytes processed using an elutriation-based procedure were superior in function to hepatocytes from the same donor liver processed using a Percoll-based procedure. More specifically, human hepatocytes processed by elutriation showed improved plateability, increased attachment efficiency, better ammonia detoxification, increased human albumin production, increased A1AT production, and higher CYP3A4 activity as compared to corresponding hepatocytes isolated from the same donor liver using a Percoll-based procedure in place of elutriation.

Analysis of the Percoll-processed and elutriated fractions also surprisingly demonstrated that elutriation reduces the presence of immune cells and inflammatory cytokines as compared to fractions processed using density gradient centrifugation using Percoll. For example, FIG. 7 shows the relative expression levels of mRNAs encoding inflammatory cytokines, interleukin 1-beta (IL-1beta), interleukin 6 (IL-6), tumor necrosis factor alpha (TNFalpha), and tumor necrosis factor beta (TNFbeta), in fractions of human hepatocyte lots 1, 2, and 3 processed using either Percoll density gradient centrifugation (“P”) or elutriation (“E”). As can be seen, in each pairwise comparison of Percoll-processed fraction versus corresponding elutriation-processed fraction expression of each inflammatory cytokine was lower in the elutriated fraction. Moreover, as displayed in Table 3, flow cytometric analysis of Percoll-processed and elutriated fractions for immune-cell marker expressing cells showed that less CD45+ cells were present in the elutriated fractions as compared to the corresponding Percoll-processed fractions.

TABLE 3

| Percent of cell population CD45+ | Lot 1 | Lot 2 | Lot 3 |
|---|---|---|---|
| Percoll-processed fraction | 7.47% | 1.72% | 1.27% |
| Elutriation-processed fraction | 1.3% | 0.85% | 0.04% |

Collectively, these data demonstrate that elutriation-based processing is unexpectedly more effective than Percoll-based density gradient centrifugation at removing nonparenchymal cells, such as CD45 expressing immune cells, and also results in processes cell fractions that contain less inflammatory cytokines than comparable Percoll-processed fractions.

Collectively, these findings demonstrate that elutriation can be effectively employed to enrich cellular samples, including xenomixtures containing human hepatocytes and non-human cells (such as non-human hepatocytes and/or other non-human cells, such as non-human NPCs) and human-only mixtures of different human cell types, for desired human hepatocytes. Accordingly, using elutriation, cell processing procedures can be employed to increase the purity and the enrichment of human hepatocytes, e.g., as compared to corresponding procedures that do not employ elutriation and/or employ dentistry gradient centrifugation in place of elutriation. Elutriation is also useful for removing NPCs and, e.g., for generating cell preparations that have reduced levels of immune cells and/or immune cell products, such as inflammatory cytokines, as compared to corresponding procedures that do not employ elutriation. Moreover, the human hepatocytes isolated using an elutriation-based procedure demonstrate enhanced functional characteristics, e.g., as compared to human hepatocytes isolated from corresponding procedures that do not employ elutriation such as, e.g., Percoll-based procedures. Without being bound by theory, the superior fitness and potency of human hepatocytes isolated using elutriation, e.g., as compared to those isolated using procedures that employ centrifugal sedimentation (i.e., “pelleting”) of hepatocytes which subjects the cells to compaction and stress, showed that the use of elutriation is more gentle on the eventually isolated hepatocytes resulting in a final enriched cell population that is substantially improved as compared to a corresponding population isolated using conventional methods.

### Example 12—Identification of Pan-Pig-Specific Antibodies Sufficient for Enrichment of Human Hepatocytes from a Xenomixture

As described in Example 9, the rat homolog of human major histocompatibility complex class I (MHCI)/HLA class I histocompatibility antigen was found to be a useful antigen for purification by negative selection of xenomixtures containing rat cells and desired human hepatocytes. Accordingly, the corresponding swine homolog was investigated for use as a target antigen for purification by negative selection of xenomixtures containing pig cells and desired human hepatocytes.

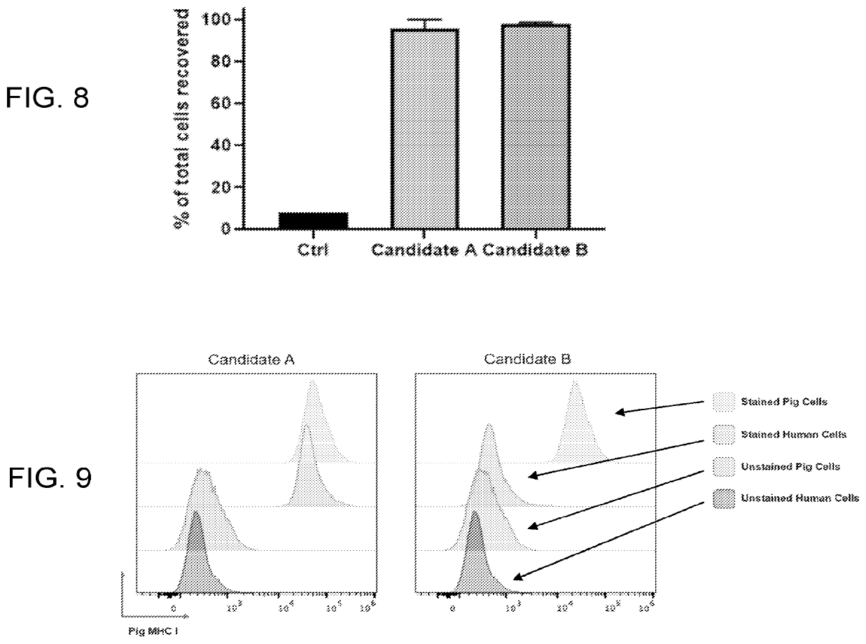

Monoclonal antibodies specific for swine MHC class I antigen, also referred to as swine leukocyte antigen 1 (SLA-1) were screened for binding to pig cells broadly. Antibody candidates identified as pan-pig-specific antibodies were tested in a recovery assay to assess the use of each antibody for retaining and recovering pig cells. Briefly, in one example, cell populations containing a heterogenous mixture of pig cells were incubated with either control buffer containing no antibody (“Ctrl”), anti-SLA-1 candidate antibody clone A (“Candidate A”), or anti-SLA-1 candidate antibody clone B (“Candidate B”). After primary antibody, or control, incubation each sample was incubated with a magnetic-bead-bound secondary antibody. Next, the antibody-containing xenomixture was followed through a column with a magnetic force applied to the column, and the initial flow-through was discarded. The magnetic force was then removed and the columns were washed, collecting the subsequent flow-through, which was then evaluated by cell count and measuring the proportion between the retained and the total number of cells between the retained and flow through fractions to generate the percent recovery of the total number of pig cells applied to the column. Results are provided in FIG. 8, which shows that both candidate anti-SLA-1 antibodies A and B were effective at retaining pig cells by magnetic-based selection. The control (“Ctrl”) indicated minimal retention of pig cells in the absence of an anti-SLA-1 antibody.

The candidate antibodies were further evaluated for cross-reactivity with human cells, where significant binding of the antibodies to human hepatocytes would indicate unsuitability for use in a procedure for purifying human hepatocytes from a pig cell-containing xenomixture by magnetic negative selection. A flow cytometric-based assay was used to assess binding of Candidate A and Candidate B antibodies to pig and human cells. As can be seen in FIG. 9, where fluorescence measured on the x-axis indicates binding of SLA-1 (also referred to as “Pig MHC I”), antibody Candidate A (left panel) showed binding to pig cells (“Stained Pig Cells”), but also showed significant cross-reactivity with human cells (“Stained Human Cells”) whereas antibody Candidate B (right panel) showed binding to pig cells, but insignificant cross-reactivity with human cells. Accordingly, Candidate B was determined to be the more suitable candidate for use in purifying human hepatocytes from a xenomixture containing pig cells. Cells not incubated with either candidate antibody (“Unstained Pig Cells” and “Unstained Human Cells”) were used as negative controls.

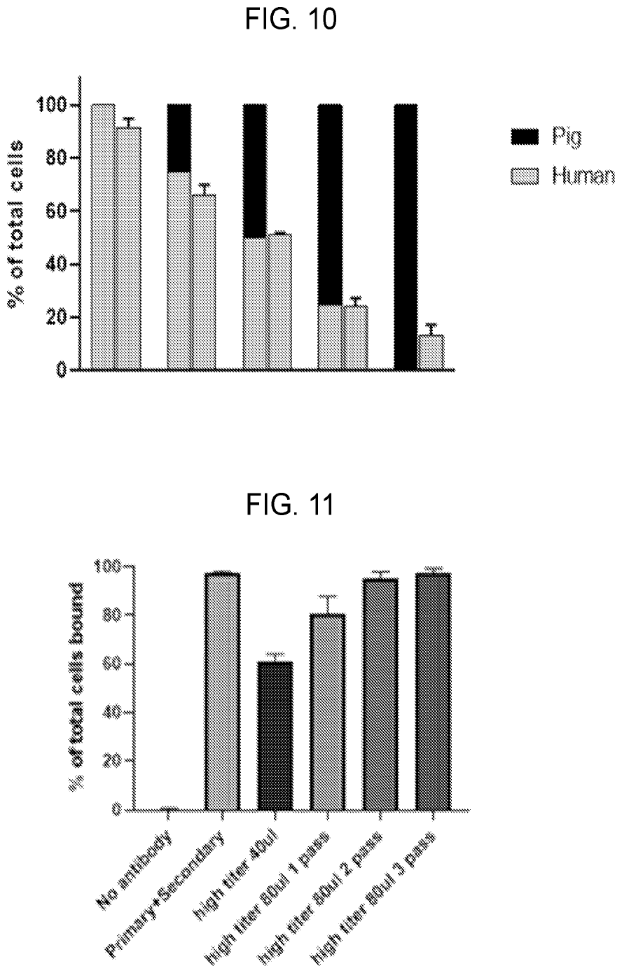

Anti-SLA-1 candidate antibody “B” was further used in trial purification assays to test the effectiveness of the antibody for use in purifying human hepatocytes from xenomixtures of human and pig cells by magnetic bead-based negative selection. Briefly, xenomixtures containing 100% human cells, 75% human cells (25% pig cells), 50% human cells (50% pig cells), 25% human cells (75% pig cells), and 100% pig cells were prepared, incubated with antibody, and subjected to magnetic bead-based selection of pig cells followed by flow cytometric analysis. FIG. 10 shows the input ratio of human to pig cells (left bar of each pair) and the percent of the total input cells obtained in the flowthrough that were human (right bar of each pair). In each xenomixture ratio tested, despite retention of pig cells in the magnetized column, all or nearly all of the input human cells were retrieved.

Collectively, these data demonstrate the identification of a pan-pig antibody that is sufficiently specific for purification of human hepatocytes from a human-swine xenomixture by magnetic bead-based negative selection procedure. The data further demonstrates the use of this antibody specifically, and anti-pig antibodies generally, in the context of human hepatocyte collection from xenomixtures. These results support that the human hepatocyte enrichment procedures described herein may be employed with various different xenomixtures containing human and non-human cells, including e.g., a human-swine xenomixture such as is obtained from a fully or partially humanized swine liver, such as e.g., a Fah-deficient swine liver that has been at least partially repopulated with transplanted human hepatocytes.

### Example 13—Large-Scale Human Hepatocyte Enrichment Using a Closed System

This example describes the use of a closed system and process for the large-scale collection, enrichment, and purification of human hepatocytes from a xenomixture. The xenomixture was obtained by the perfusion of multiple humanized livers harvested from huFRG rat in vivo bioreactors into which human hepatocytes were introduced, engrafted, and expanded essentially as described in the preceding examples. At least 500 mL of liver perfusate xenomixture was collected into a 1-2 L sterile custom GMP manufactured transfer bottle or 1-3 L transfer bag with attached sterile transfer tubing. The perfusate-filled bottle or bag was sterile tube-welded to a sterile GMP manufactured cell processing kit that includes sterile connections for elutriation and separate collection and waste lines/containers.

All reagent transfers, including buffers, etc., described in this example were performed using sterile disposable GMP transfer bottles/bags and all intermediate and final collection containers used were sterile disposable GMP transfer bottles/bags. In some instances, all reagents used were animal-origin-free including, e.g., the elutriation buffer which was plasmalyte A and human serum albumin (HSA) based. Elutriation to enrich for human hepatocytes was performed using dual 50 mL elutriation chambers on an elutriator running an automated elutriation program with a run time of less than 30 minutes to clear the dual 50 mL chambers. Human hepatocyte-containing elutriated fractions totaling about 100 mL of cell suspension volume were pooled into a single transfer bag/bottle.

Anti-RT1A antibody (either with magnetic secondary antibody or directly conjugated to magnetic bead) was incubated with the cells of the human-hepatocyte-enriched xenomixture, either in a culture container or after sterile introduction into the aforementioned transfer bag/bottle. In some instances, e.g., where antibody incubation is performed in a transfer bag, the cell and antibody suspension is mixed with an independent rocker platform or on a magnetic-plate containing panel operably connected to an actuator. Following incubation, the purified human hepatocytes were collected, e.g., through the attachment of sterile tubing to the container.

Purified human hepatocytes were mixed with cryopreservation reagents to obtain desired final concentrations of cells in cryopreservation media. 100 mL of hepatocytes at a 2× dose concentration, in cryopreservation media, were transferred, using sterile tubing, into a 50 mL to 750 mL cryopreservation bag (“cryobag”) suitable for use with one or more bag thawing devices. Separate method development indicated, unexpectedly, that the human hepatocyte viability in cryobags containing a 2× concentration of cells (e.g., 20E6 cell/mL) was very similar, without a significant decrease in viability, to the 1× concentration (e.g., 10E6 cells/mL) assumed to be optimal for hepatocyte viability from smaller scales. For example, comparing 1× to 2× concentrations revealed viability of 73.1% (at 1×) vs. 66.9% (at 2×) in 1.5 mL vials, a greater than 5% decline, as compared to 69.9% vs. 70.7% in 150 mL cryobags at 1× and 2× concentrations, respectively. This finding not only facilitates greater flexibility in dosing but also allows for the closed-system preparation of a single 750 mL dose bag from 100 mL of cell suspension at a 1:4 dilution factor with over 1E9 live cells.

### Example 14—Magnetic Selection Using Magnetic Bead Conjugated Anti-Rat RT1A Class I Histocompatibility Antigen Antibody

An anti-RT1A antibody, essentially as described in Example 9, was directly conjugated to 0.05-micron beads according to standard procedures to generate high and low titer direct-magnetic-bead conjugated anti-RT1A antibody. Binding of the directly conjugated antibody to target rat cells, indicating recovery of cells bound in a magnetic separation procedure, was assessed.

Briefly, aliquots of target rat cells were incubated in a no antibody negative control (“no antibody”), with unconjugated primary and secondary antibodies in a positive control as described above (“Primary+Secondary”), or with varied amounts of low or high titer directly conjugated antibody. Following incubation, each aliquot was passed through a magnetic binding column with a magnet applied. The columns were subsequently washed with the magnet removed, the flow-through was collected, and the percent of the total cells recovered in the flow-through, as indicative of the percent of total cells bound by the column, was determined.

As shown in FIG. 11, the directly conjugated antibody proficiently bound and sequestered the target cells in the magnetized column with 40 μl and 80 μl of high titer antibody retaining at least 60% and 80% of the target cells, respectively, after one pass of cell suspension through the column (see “high titer 40 μl” and “high titer 80 μl 1 pass”). Moreover, even under conditions not yet optimized for use with the directly conjugated antibody, the percent of total target cells bound by the column after multiple passes through the column of 80 μl of high titer antibody-treated cell suspension was comparable to the amount of target cells bound by the more optimized primary-secondary antibody procedure (compare e.g., “high titer 80 μl 2 pass” and “high titer 80 ul 3 pass” to “Primary+Secondary”). These data demonstrate the ability to use a directly conjugated magnetic anti-RT1A primary antibody in place of the primary and magnetic secondary antibody approach as described above. In addition, these data demonstrate that this procedure can be employed to retain high percentages (e.g., at least 60%, at least 80%, greater than 90%, nearly 100%, etc.) of the target cells by magnetic negative selection, further validating this approach, and the ability to use magnetic secondary antibodies or directly conjugated magnetic primary antibody, for the isolation of human hepatocytes from a xenomixture.

### Example 15—Processing of Cadaveric PHH Results in Isolated Expanded Populations of Human Hepatocytes with Distinct Gene Expression and Favorable In Vivo Functional Characteristics

Populations of expanded hepatocytes from FRG rat bioreactors processed and isolated according to the methods described herein (including, e.g., enrichment by elutriation and purification by antibody-based negative selection) were further compared to healthy unexpanded cadaveric hepatocytes to assess similarities and differences between the cell populations. For example, single cell gene expression was assessed by single-cell RNA-Seq in unamplified cadaveric PHH populations from two different donors (“PHH Donor A” and “PHH Donor B”) and compared to single-cell RNA-Seq performed in two populations of huFRG expanded and isolated hepatocytes that had been separately sourced, expanded in FRG rat bioreactors, processed, and isolated as described herein (“huFRG Human hepatocytes A” and “huFRG Human hepatocytes B”).

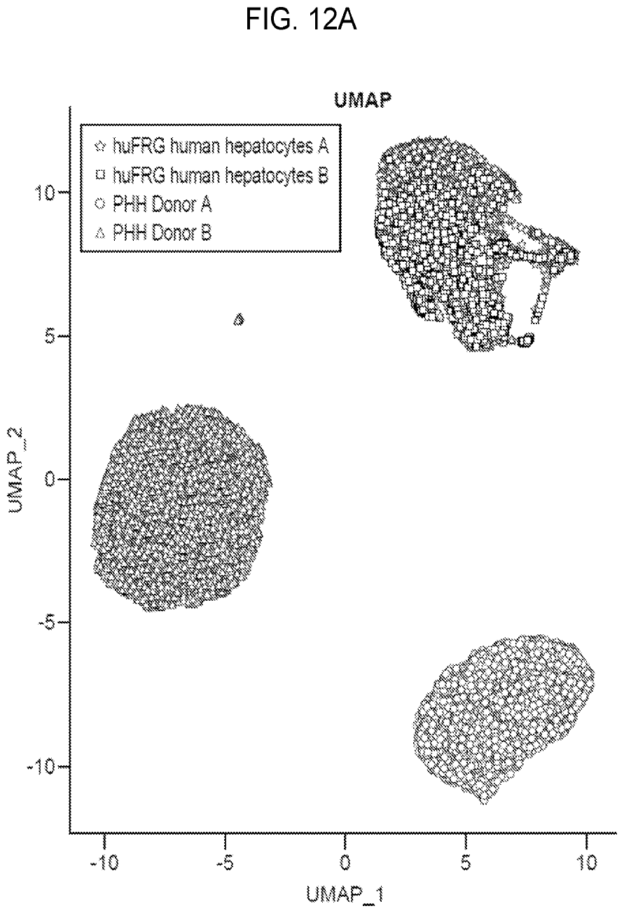

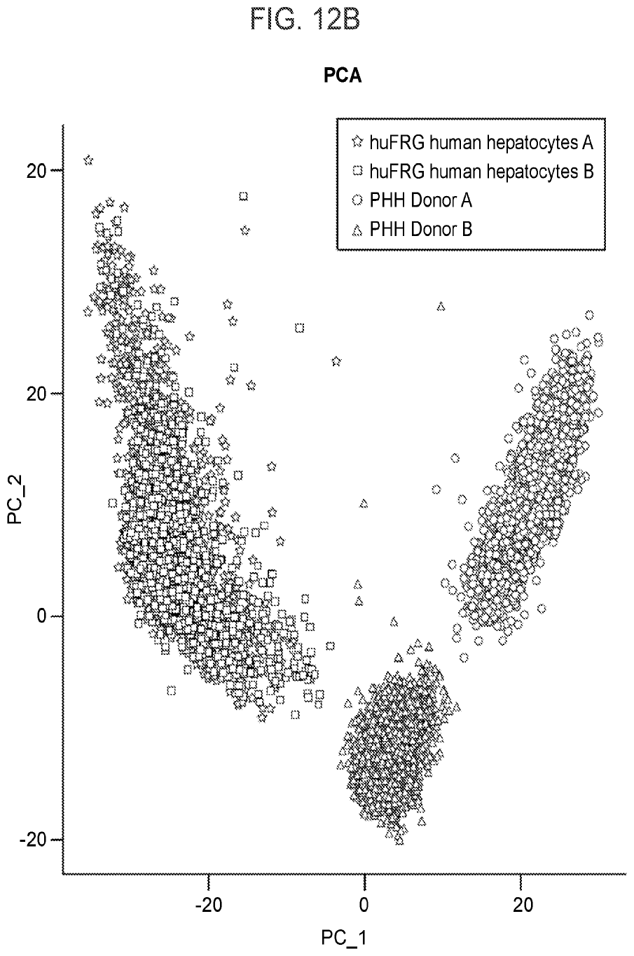

Global gene expression pattern analysis produced from the single-cell RNA-Seq for each population is provided in FIGS. 12A and 12B, rendered as a Uniform Manifold Approximation and Projection (UMAP) plot and a principal component analysis (PCA), respectively. In this context, the UMAP and PCA plots simplify and transform the highly complex single-cell RNA-Seq data while retaining trends and patterns to visually demonstrate how similar or dissimilar the cell populations are with respect to one another. As shown in both the UMAP and PCA plots, the data points making up the two huFRG human hepatocyte populations cluster and/or overlap whereas both PHH donor populations plot separately from the huFRG populations. This data demonstrates that the huFRG human hepatocyte populations, having both been expanded in the FRG bioreactor and processed and isolated in the same way, are more similar to one another than they are to either of the unexpanded cadaveric PHH populations. Accordingly, this data also highlights surprising differences in global gene expression between human hepatocytes before versus after expansion and processing as well as surprising similarity between the isolated expanded human hepatocyte populations. These data demonstrate that the ultimately produced isolated expanded human hepatocytes are characteristically different from the PHH prior to expansion (i.e., as sourced from human liver).

The in vivo function of human hepatocytes before vs. after bioreactor expansion and processing as described herein was also compared by transplanting the cells into receptive host animals and assessing repopulation. Briefly, PHH were collected from a cadaveric donor liver and cryopreserved in multiple aliquots. A portion of the aliquoted cells were thawed and expanded in the FRG rat bioreactor, then the expanded cells were processed by elutriation and isolated via anti-RT1A antibody-based negative selection to generate an expanded and isolated population of human hepatocytes. Next, equivalent numbers of the unexpanded cadaveric donor PHH (i.e., “Cadaveric PHH”) and the isolated expanded human hepatocytes (i.e., “huFRG human hepatocytes”) were transplanted into recipient FRGN mice. The mice were maintained under conditions sufficient for expansion and repopulation of the host livers by the transplanted cells.

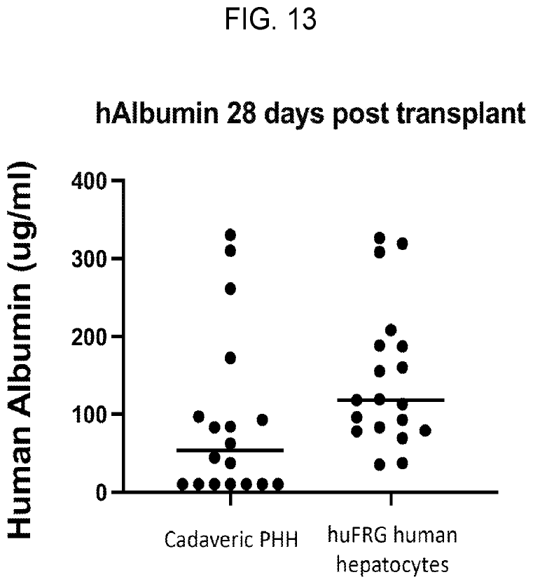

Levels of human albumin (hAlb, micrograms/milliliter) were measured by ELISA in blood samples collected from mice of both groups. Results showing the hAlb levels at 28 days post-transplant in mice that received either Cadaveric PHH or huFRG human hepatocytes are provided in FIG. 13. As shown, the mean level of hAlb (horizontal bar in each data series) was higher in the blood samples from the huFRG human hepatocyte-transplanted animals as compared to the levels measured in animals that received the Cadaveric PHH. These data indicate that, despite being derived from the same donor liver, the isolated expanded huFRG human hepatocytes displayed superior function in vivo as compared to the Cadaveric PHH that were neither expanded nor processed as described herein. The FRGN mouse represents an immune-deficient mouse model of hereditary tyrosinemia type 1 (HT1 mice). As shown, the huFRG cells proliferated at significantly increased kinetics compared to cadaveric PHH cells, repopulating the mouse livers at enhanced levels. Importantly, the huFRG cells functioned in vivo for greater than 4 months and normalized tyrosine and succinylacetone levels which are characteristically elevated in the HT1 model which recapitulates the human disease phenotype. Ultimately, the transplanted huFRG human hepatocytes prevented the onset of liver failure, the terminal disease phenotype in this HT1 model.

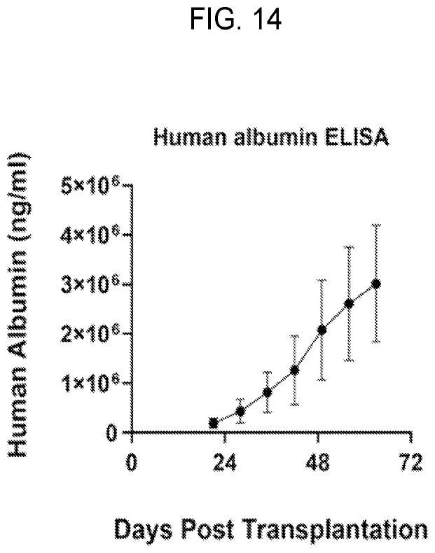

In a separate experiment, huFRG human hepatocytes were transplanted into another immune-deficient mouse model with liver injury to assess engraftment, expansion, and in vivo function of the huFRG cells outside the context of hereditary tyrosinemia. Specifically, cDNA-uPA/SCID recipient mice (PhoenixBio) were each transplanted with between 0.5×10⁶ to 1.0×10⁶ previously cryopreserved huFRG human hepatocytes by intrasplenic injection and the animals were assessed over the course of 63 days. Blood samples were collected and whole blood hAlb concentrations were measured at multiple timepoints by latex agglutination immunonephelometry. As shown in FIG. 14, hAlb concentrations increased over the course of the study, indicating that the transplanted huFRG human hepatocytes were functional and capable of engrafting and expanding in the cDNA-uPA/SCID recipient mice. These data demonstrate that huFRG human hepatocytes engraft, expand, and remain functional when transplanted into various recipient animals, even when those recipient animals carry a wildtype Fah gene (i.e., the recipient is not Fah-deficient) unlike the FRG animals in which the huFRG hepatocytes were originally expanded. Thus, huFRG cells are capable of engraftment and expansion in recipient hosts generally, including diseased hosts and disease models other than HT1.

Collectively, the results described in this example demonstrate that human hepatocytes generated through expansion and processing as described herein are characteristically different, e.g., by global gene expression analysis, from the cadaveric cells from which they were derived. Moreover, these results also demonstrate the surprising finding that, using the processing methods described (including e.g., expansion, enrichment, and isolation) to produce the isolated expanded populations of human hepatocytes, results in cells that are at least functionally equivalent, if not superior, in the in vivo context to the cadaveric cells from which they were derived. In addition, the data demonstrates that the engraftment and expansion of functional human hepatocytes generated through the methods as described herein is not limited to the contexts of Fah-deficient host animals or animal models of HT1. Rather, human hepatocytes generated through expansion and processing as described herein engraft, expand, and perform normal hepatocyte functions (such as the production of hAlb) following transplantation into varied recipients.

### Example 16—HuFRG Hepatocytes are Superior to Immortalized Hepatocyte Cell Lines and Hepatocyte-Like Cells (HLCs)

In this example, the superiority of huFRG cells over other hepatocytes and hepatocyte-like cells (HLCs) is demonstrated. In particular, huFRG cells were compared in vivo to immortalized hepatocyte cancer cell lines, such as HepaRG (ThermoFisher/GIBCO) and HepG2 (ATCC), and de novo generated HLCs (FUJIFILM Cellular Dynamics, Inc.) derived from iPSCs. Each of these various cell types were transplanted into HT1 mice recipients and the mice were assessed for engraftment, expansion, and hepatocyte function. Only mice that received transplantation of huFRG human hepatocytes demonstrated engraftment, proliferation, expansion, and substantial function of the transplanted cells in vivo. The assessment of functional parameters included ammonia detoxification, assessed by challenging the subject hepatocytes with ammonia and measuring the amount of ammonia remaining after an incubation time. Briefly, the subject hepatocytes were plated in maintenance media, the maintenance media was replaced with media containing ammonium chloride, and the hepatocytes were further incubated at 37 deg. C and 5% carbon dioxide for 3 hours. Following incubation, the amount of ammonia present was quantified against a standard curve using an ammonia quantification kit (FUJIFILM Wako Chemicals, USA). The percent ammonia detoxification was calculated as the difference between the Ammonia Challenge Control (ACC) concentration and the sample concentration divided by the ACC concentration.

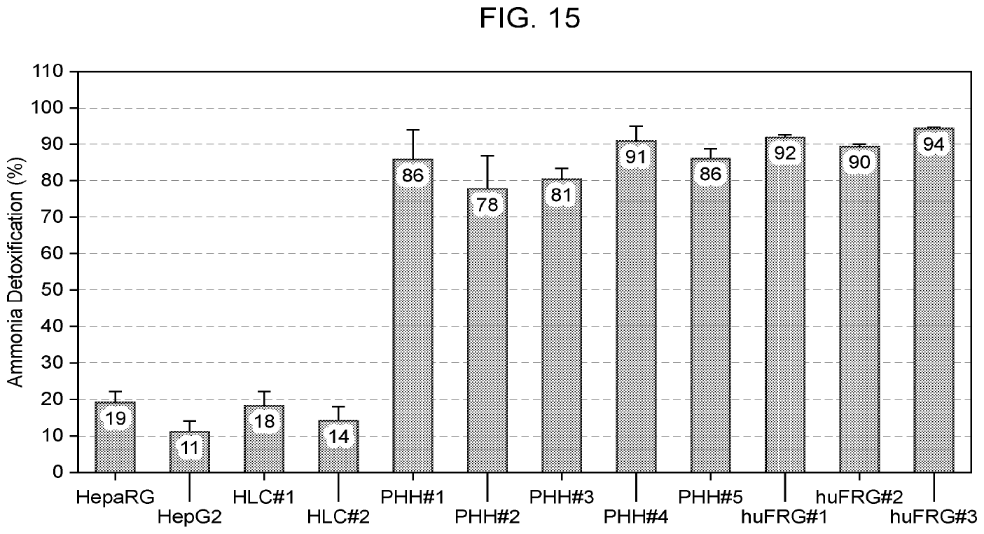

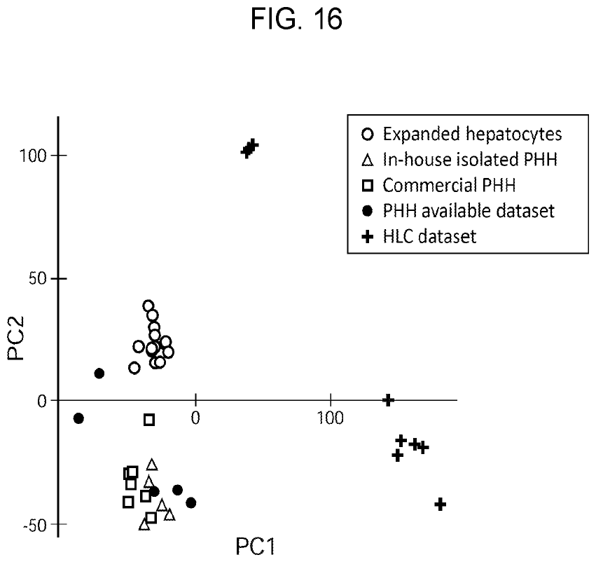

As shown in FIG. 15, huFRG cells (huFRG #1, huFRG #2, huFRG #3) showed superior ammonia detoxification, resulting in 90% to 94% ammonia detoxification, as compared to HLCs (HLC #1 and HLC #2, 18% and 14% ammonia detoxification respectively) and immortalized hepatocyte cell lines (HepaRG and HepaG2, 19% and 11% ammonia detoxification respectively). Moreover, the ammonia detoxification observed in the huFRG cells was comparable, if not superior, to the ammonia detoxification observed in cadaveric primary human hepatocytes, ranging from 78% to 91% ammonia detoxification (see also FIG. 15, PHH #1-PHH #5). As such, in these same metrics, huFRG hepatocytes generated using the methods described herein are at least comparable to cadaveric PHH. Collectively, these results demonstrate that huFRG hepatocytes generated using the methods described herein are superior in engraftment, proliferation and expansion, and hepatic function, including ammonia detoxification, to cell types that have been purported to be potential alternatives to primary and expanded hepatocytes, such as HLCs and immortalized hepatocyte cell lines.

### Example 17—Gene Signatures of Expanded Human Hepatocytes

To further compare populations of expanded human hepatocytes generated as described herein with unexpanded PHH, the genetic profiles of the expanded cells were compared to the genetic profiles of PHH, using bulk RNA sequencing which uses gene expression to identify and define different hepatocyte populations. In this analysis, commercially available PHH lots, which are readily available, were used for comparison to the expanded human hepatocytes. In addition, previously published bulk RNA datasets of both PHH and HLCs (generated from iPSCs) were also used for comparison (see Du, et al. (2014) Cell Stem Cell; Li, et al. (2021) Stem Cell Reports; and Gupta, et al. (2021) Archives of Toxicology).

To compare the genetic profiles of the various cell populations a PCA approach was used, which simplifies the complexity in the high-dimensional data (e.g., gene expression data) while retaining trends and patterns. PCA reduces all gene expression information into fewer axes (PCs) that account for most of the variation in the data and are useful for summarizing sample similarity. For example, a PCA plot that includes data from multiple individual batches of hepatocytes expanded and isolated as described herein (“expanded hepatocytes”), “in-house isolated PHH”, “Commercial PHH”, available PHH datasets described (“PHH available dataset”), and available iPSC derived HLC datasets described (“HLC available dataset”) is provided in FIG. 16. As the PCA plot demonstrates, the expanded hepatocytes show the closest clustering, indicating internal lot-to-lot similarity, while also clustering separately from the PHH and HLCs, indicating some differences in genetic profiles between the expanded hepatocytes and either PHH and HLCs. Notably, while the expanded hepatocytes clustered separately from the PHH groups, the expanded hepatocyte cluster was substantially closer to the PHH clusters than the HLC clusters. This indicates that expanded hepatocyte gene expression is more similar to PHH than to HLCs.

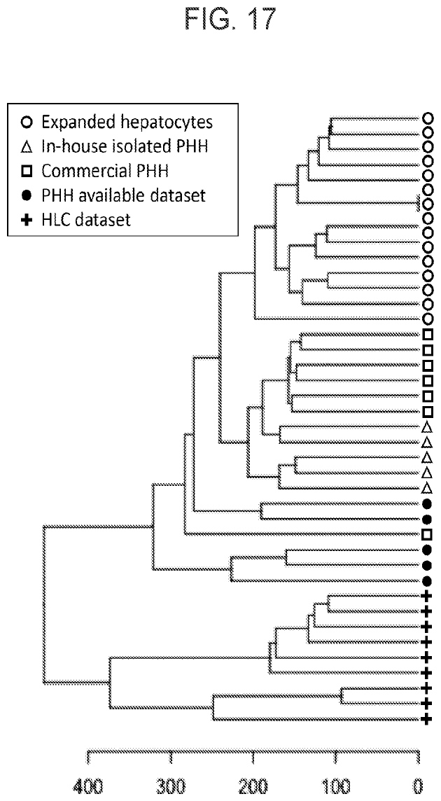

FIG. 17 provides a dendrogram generated from the bulk RNA sequencing data as used for the PCA analysis. Dendrograms summarize gene expression data, using a tree-like structure, to show relationships between samples. From left to right, the clusters (also called clades) are broken up into smaller, similar clusters until individual samples (leaves) are presented. For example, the first two subclusters represent the first two dissimilar groups, the next two subclusters represent the next two dissimilar groups, etc. This analysis shows that the expanded human hepatocyte lots indeed cluster together and are most closely related to each other and more closely related to PHH than to HLCs.

Analysis of the differentially expressed genes between the different hepatocyte sources revealed that hepatocytes expanded as described herein are similar to but yet different from both in-house isolated and commercial PHH. Thus, the expanded hepatocytes show some genetic differences from PHH; analysis of the identities of the differentially expressed genes revealed that such genetic differences were generally not due to differences in the gene expression of hepatocyte genes (i.e., genes that are normally highly expressed in, provide the functions of, or are otherwise characteristic of, hepatocytes). This is in contrast to the differences between HLCs and both PHH and expanded hepatocytes, where such differences were frequently observed in hepatocyte genes, including e.g., where hepatocyte gene expression is substantially reduced in HLCs as compared to PHH and expanded hepatocytes, including e.g., urea cycle, clotting factor, drug metabolism, serum protein binding, and bile acid synthesis gene expression.

Performing multiple PCA and other bioinformatic analyses revealed that human hepatocytes expanded as described herein demonstrate a unique gene expression signature, e.g., as compared to PHH and/or HLCs. In an analysis, the top 49 differentially up and differentially down genes were extracted from several different comparative datasets and the gene identities were extracted from each dataset and then cross-referenced. Cross-referencing the datasets identified a gene signature of differentially expressed genes that appear in multiple, if not all, datasets. Comparison of the gene signature with reference datasets (e.g., previously published PHH datasets and datasets developed from in-house isolated PHH) showed that the identified genes of the gene signature were significantly differentially expressed as compared to the gene expression in the reference datasets. Retrospective analysis revealed that gene signatures of expanded human hepatocytes are clearly differentiable from reference PHH gene expression regardless of whether the analysis was performed using bulk RNAseq or scRNAseq analyses.

Table 4 provides an example cross-referencing of multiple different PHH-to-expanded hepatocyte (EH) individual dataset comparisons of the top up-regulated genes in EH (“Bulk” and “SC” indicate whether the gene expression analysis was bulk RNAseq or scRNAseq, respectively). Table 5 provides an example cross-referencing of multiple different PHH-to-EH individual dataset comparisons of the top down-regulated genes in EH. Cross-referencing datasets, including the examples provided in Table 4 and Table 5, while taking into account the degree of fold-change up or down regulation and the statistical significance of such differences, pared-down gene signatures that identify the expanded human hepatocytes, and differentiate such cells from other hepatocytes such as PHH, were generated.

Exemplary gene signatures include combinations of one or more genes upregulated in expanded (as described herein) human hepatocyte (“EHH”) such as GPC3 (see e.g., UniProtKB P51654), AKR1B10 (see e.g., UniProtKB 060218), FXYD2 (see e.g., UniProtKB P54710), PEG10 (see e.g., UniProtKB Q86TG7), CYP7A1 (see e.g., UniProtKB P22680), and NQO1 (see e.g., UniProtKB P15559) and/or one or more genes downregulated in EHH such as C9 (see e.g., UniProtKB P02748), SAA1 (see e.g., UniProtKB PODJI8), SAA2 (see e.g., UniProtKB PODJI9), CRP (see e.g., UniProtKB P02741), NNMT (see e.g., UniProtKB P40261), SPINK1 (see e.g., UniProtKB P00995), PLA2G2A (see e.g., UniProtKB P14555), and ORM1 (see e.g., UniProtKB Q8N138). In some instances, the genes of the gene signature may be expressed above or below a suitable threshold level of expression, where such suitable levels of expression may be an absolute level of expression (e.g., above or below a particular read-count or copy number) or a relative level of expression (e.g., above or below a relative threshold in comparison to a reference expression level). Useful relative expression levels may be the level of expression of the particular gene in a reference population or cell type, such as e.g., a PHH population, an HLC population, etc., of a reference dataset, e.g., a PHH gene expression reference dataset, a HLC gene expression reference dataset, etc. Useful relative gene expression threshold include greater than 2 log fold-change up or down (sometimes expressed as positive (+) or negative (−)), greater than 2.5 log fold-change up or down, greater than 3.0 log fold-change up or down, greater than 3.5 log fold-change up or down, greater than 4.0 log fold-change up or down, greater than 4.5 log fold-change up or down, greater than 5 log fold-change up or down, greater than 5.5 log fold-change up or down, or the like.

TABLE 4

| Bulk PHH vs. EH | Bulk PHH vs. EH | | Bulk PHH vs. EH | Bulk PHH vs. EH |
|---|---|---|---|---|
| #1 | #2 | SC PHH vs. EH | #3 | #4 |
| FMO1 | AKR1B10 | GSTA1 | NA | TRIM71 |
| COL2A1 | TRIM71 | GSTA2 | FMO1 | COL2A1 |
| TRIM71 | FMO1 | TTR | CCNB2 | INAVA |
| BDKRB2 | SPATA21 | FXYD2 | CYP7A1 | SPATA21 |
| SPATA21 | INAVA | PEG10 | PEG10 | FMO1 |
| INAVA | DEPDC1 | PRAP1 | GPC3 | BDKRB2 |
| AKR1B10 | KIF4A | ALDH1A1 | LGR5 | CDH16 |
| CDH6 | COL2A1 | AKR1C1 | LINC02732 | GPC3 |
| CYP7A1 | CDH16 | GPC3 | KITLG | SLC6A11 |
| UPK3A | BDKRB2 | ADIRF | CXCL10 | CYP7A1 |
| SLC6A11 | TTK | AKR1C2 | CDH6 | SLC5A12 |
| SLC5A12 | SLC6A11 | EPHX1 | CD36 | AKR1B10 |
| DEPDC1 | SLC5A12 | AKR1C3 | INAVA | DGKK |
| GPC3 | CDC20 | AKR1B10 | NPBWR1 | CDH6 |
| CDH16 | CYP7A1 | ADH1A | CDC20 | UPK3A |
| KIF4A | TOP2A | UGT2B15 | NA | NQO1 |
| CDC20 | DGKK | CYP7A1 | MELK | MSI1 |
| DGKK | GPC3 | ADH4 | UGT2B11 | KIF4A |
| MSI1 | DLGAP5 | S100A10 | MSI1 | ABO |
| TOP2A | ABO | UGT2B10 | AKR1B10 | DEPDC1 |
| CKAP2L | MCM10 | ENO3 | BUB1 | CDC20 |
| NPBWR1 | NPBWR1 | FETUB | SPATA18 | FXYD2 |
| FAM111B | FAM111B | PHYH | FRAS1 | PEG10 |
| DLGAP5 | CDH6 | UGT2B7 | EDA2R | SGIP1 |
| TTK | NQO1 | DEFB1 | TYMSOS | GRAMD1B |
| MCM10 | KIF20A | FTL | SLC5A12 | RHOBTB1 |
| CXCL10 | CCNB2 | IL32 | RHOBTB1 | DLGAP5 |
| NQO1 | CKAP2L | FTH1 | CCDC3 | FBLN1 |
| CD36 | KIF18B | OTC | KIF20A | TOP2A |
| TYMSOS | UPK3A | MGST1 | FXYD2 | CDK1 |
| CCNB2 | CDK1 | ALB | SMAD9 | E2F8 |
| SGIP1 | MSI1 | LECT2 | SHC3 | TTK |
| DIAPH3 | HMMR | BCHE | FGF2 | ADAMTS16-DT |
| PEG10 | CENPA | ACAA1 | TOP2A | KIF18B |
| FBLN1 | FAM72D | ENSG00000230202 | MYBL2 | CD36 |
| CDK1 | DIAPH3 | TXN | CDH16 | FAM111B |
| DEPDC1B | UHRF1 | TM4SF4 | NA | DIAPH3 |
| FXYD2 | MKI67 | FABP1 | CENPM | SHC3 |
| KIF18B | TYMSOS | PEBP1 | CENPA | SLC2A14 |
| TYMS | E2F8 | HULC | BEX1 | E2F7 |
| ADAMTS16-DT | CCNA2 | SERPINA5 | SSTR2 | UHRF1 |
| MKI67 | CDC25C | FDPS | FNDC5 | SIGLEC15 |
| PBK | FBLN1 | APOC2 | FCAMR | CCNB2 |
| GPSM2 | KIF23 | GPX4 | CNNM1 | DEPDC1B |
| GRAMD1B | PBK | APOA2 | NQO1 | CXCL10 |
| CENPA | SGIP1 | CXCL10 | TROAP | ADAMTS16 |
| CCNA2 | GPSM2 | PRXL2A | PHLDA3 | PBK |
| KITLG | UGT2B11 | FAM162A | CLDN19 | CENPF |
| HMMR | DEPDC1B | ADH1B | RAB38 | NDC80 |

TABLE 5

| Bulk PHH vs. EH | Bulk PHH vs. EH | | Bulk PHH vs. EH | Bulk PHH vs. EH |
|---|---|---|---|---|
| #1 | #2 | SC PHH vs. EH | #3 | #4 |
| C9 | C9 | SAA1 | FGF14 | C9 |
| CRP | IFITM1 | SAA2 | C9 | SAA2-SAA4 |
| PLA2G2A | SAA2-SAA4 | HP | PLA2G2A | NNMT |
| NNMT | NNMT | NNMT | NNMT | C1QB |
| SAA2-SAA4 | KNDC1 | CRP | CRP | C1QA |
| SPINK1 | PLA2G2A | ORM1 | LINC01702 | IGHM |
| LCN2 | APOA4 | C9 | RUNX3 | C1QC |
| C1QB | HLA-DPA1 | FGL1 | NA | FGF14 |
| APOA4 | C1QB | SERPINA3 | C1QA | PLA2G2A |
| C1QA | C11orf96 | HAMP | RGS1 | IFITM1 |
| C11orf96 | LCN2 | HSPA1A | VSIG4 | HLA-DRA |
| SAA2 | IGHM | TIMP1 | SAA2-SAA4 | LINC01702 |
| MARCO | C1QC | SPINK1 | C1QB | MARCO |
| IFITM1 | CRP | PLA2G2A | SAA1 | CRP |
| SAA1 | FAM83A-AS1 | CP | SPINK1 | HLA-DPA1 |
| C1QC | H4C11 | LRG1 | SAA2 | IGHG1 |
| KNDC1 | CXCR4 | ORM2 | IRF4 | C11orf96 |
| VSIG4 | DNAH5 | IGFBP2 | HLA-DRA | RGS1 |
| AVPR1A | AHNAK2 | SERPING1 | CHRDL2 | AVPR1A |
| SIGLEC1 | IL10RA | IFITM2 | C3P1 | MPEG1 |
| MPEG1 | CCL4 | NAMPT | HLA-DPB1 | VSIG4 |
| DNAH5 | C1QA | HSPA1B | CCL3 | C3P1 |
| LINC01702 | AVPR1A | ATF5 | TOX | KNDC1 |
| TOX | LINC01702 | CFHR3 | C1QC | SPINK1 |
| PAPPA2 | MS4A7 | ERRFI1 | LINC01554 | SIGLEC1 |
| HLA-DRA | SPINK1 | C4BPA | CHI3L1 | DNAH5 |
| C3P1 | CPLX1 | DNAJB1 | MSR1 | LINC01554 |
| CPLX1 | PAPPA2 | CXCL2 | SRGN | IGKC |
| HLA-DPA1 | IGHG1 | H19 | IGHM | SELE |
| SOCS2 | TOX | SDS | S100P | IGHA1 |
| S100P | ORM1 | APCS | MPEG1 | HLA-DPB1 |
| CD5L | MARCO | SERPINA1 | C5AR1 | SRGN |
| IGHM | RUNX3 | ITIH4 | CCL4 | SAA2 |
| FGF14 | LINC02158 | C1R | AVPR1A | SAA1 |
| CSF1R | CSF3R | CEBPD | C11orf96 | C5AR1 |
| CCL4 | C3P1 | TDO2 | CD53 | CXCR4 |
| ORM1 | IGKC | FGA | S100A9 | CCL4 |
| C5AR1 | SPI1 | AGT | HCK | CD5L |
| SELE | S100P | LBP | DOK2 | FAM83A-AS1 |
| SRGN | MPEG1 | ST6GAL1 | MARCO | IL10RA |
| CCL3 | C5AR1 | GADD45G | FGR | MS4A7 |
| TMC5 | TYROBP | HPX | LCN2 | RUNX3 |
| AHNAK2 | HLA-DRA | IGFBP1 | IGHG1 | PTPRC |
| LINC02158 | SOCS2 | C8G | CSF1R | TOX |
| CXCR4 | TMC5 | IFITM3 | IGF1 | CPLX1 |
| LINC01554 | SIGLEC1 | TNFSF14 | NA | CSF1R |
| MS4A7 | KCNB1 | G0S2 | IGHG2 | SOCS2 |
| IL10RA | CYBA | CFH | CXCR4 | CCL3 |
| CD53 | SAA1 | FGB | SIGLEC1 | CD53 |

This example demonstrates that while EHH are different from both commercially sourced and in-house perfused PHH, such cells are more similar to PHH than to HLCs derived from iPSCs. Moreover, this example shows that EHH can be defined and identified by gene expression signatures. Such gene expression signatures can be used for various purposes, including to define EHH cell populations, identify EHH cells, differentiate EHH from other cell populations (e.g., PHH, HLC, etc.), and the like. Other uses include characterization of, development of, and/or quality control over cell production procedures, including e.g., when involved in methods to assess reproducibility of generated expanded human hepatocyte cell populations.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of producing isolated expanded human hepatocytes, the method comprising:
collecting hepatocytes from the liver of a non-human in vivo bioreactor in which human hepatocytes have been expanded, wherein the collected hepatocytes comprise a xenomixture of the expanded human hepatocytes and non-human in vivo bioreactor hepatocytes; and
A) subjecting the xenomixture to centrifugal elutriation and then removing non-human in vivo bioreactor cells from the elutriated xenomixture via a negative selection process to produce isolated expanded human hepatocytes; or
B) removing non-human in vivo bioreactor cells via a negative selection process and then performing centrifugal elutriation to produce isolated expanded human hepatocytes.

2. A method of producing isolated expanded human hepatocytes, the method comprising:
introducing human hepatocytes into the liver of a non-human in vivo bioreactor;
expanding the human hepatocytes in the liver of the non-human in vivo bioreactor;
collecting hepatocytes from the liver of the non-human in vivo bioreactor, wherein the collected hepatocytes comprise a xenomixture of expanded human hepatocytes and non-human in vivo bioreactor hepatocytes;
subjecting the xenomixture to centrifugal elutriation under conditions sufficient to produce an elutriation fraction enriched for the expanded human hepatocytes; and
removing non-human in vivo bioreactor cells from the elutriation fraction via a negative selection process to produce isolated expanded human hepatocytes.

3. The method according to claim 2, wherein introducing the human hepatocytes into the liver of the non-human in vivo bioreactor comprises delivering the human hepatocytes to the spleen of the non-human in vivo bioreactor.

4. The method according to claim 3, wherein delivering the human hepatocytes to the spleen of the non-human in vivo bioreactor is by splenic injection.

5. The method according to any of the preceding claims, comprising monitoring the expansion of the human hepatocytes in the liver of the non-human in vivo bioreactor.

6. The method according to claim 5, wherein the monitoring comprises monitoring the level of a circulating biomarker secreted by the human hepatocytes in the non-human in vivo bioreactor during the expanding.

7. The method according to claim 6, wherein the circulating biomarker is human albumin (hAlb).

8. The method according to claim 6 or claim 7, wherein the level of the circulating biomarker is monitored in whole blood obtained from the non-human in vivo bioreactor.

9. The method according to any one of claims 5 to 8, wherein collecting hepatocytes from the liver of the non-human in vivo bioreactor commences based on the monitored level of the circulating biomarker reaching a threshold level.

10. The method according to any one of claims 1 to 9, wherein collecting hepatocytes from the liver of the non-human in vivo bioreactor commences based on a clinical score cutoff being met.

11. The method according to any one of claims 1 to 10, wherein the expanded human hepatocytes constitute 50% or greater, 60% or greater, or 70% or greater of the total cells present in the elutriation fraction.

12. The method according to any one of claims 1 to 11, wherein the non-human in vivo bioreactor is deficient for fumarylacetoacetate hydrolase (Fah).

13. The method according to claim 12, wherein expanding comprises 2-(2-nitro-4-trifluoromethylbenzoyl)-1,3-cyclohexanedione (NTBC) cycling.

14. The method according to any one of claims 1 to 13, wherein the non-human in vivo bioreactor is a rodent in vivo bioreactor.

15. The method according to claim 14, wherein the rodent in vivo bioreactor is a rat in vivo bioreactor.

16. The method according to claim 14 or claim 15, wherein the rodent in vivo bioreactor is deficient for interleukin 2 receptor subunit gamma (IL2rg), recombination activating gene 1 (RAG1), recombination activating gene 2 (RAG2), or a combination thereof.

17. The method according to any one of claims 1 to 13, wherein the non-human in vivo bioreactor is a pig in vivo bioreactor.

18. The method according to any one of claims 1 to 17, wherein the negative selection process is an antibody-based negative selection process.

19. The method according to claim 18, wherein the antibody-based negative selection process comprises:
contacting the elutriation fraction, the elutriated xenomixture, or the xenomixture with a primary antibody specific for non-human in vivo bioreactor cells under conditions sufficient for specific binding of the primary antibody to non-human in vivo bioreactor cells present in the elutriation fraction, elutriated xenomixture, or xenomixture; and
removing non-human in vivo bioreactor cells from the elutriation fraction, elutriated xenomixture, or xenomixture utilizing the primary antibody.

20. The method according to claim 19, wherein removing non-human in vivo bioreactor cells utilizing the primary antibody comprises contacting the primary antibody with a labeled secondary antibody under conditions sufficient for binding of the secondary antibody to the primary antibody, and utilizing the label of the labeled secondary antibody to remove, from the elutriation fraction, elutriated xenomixture, or xenomixture, complexes comprising labeled secondary antibody, primary antibody, and a non-human in vivo bioreactor cell.

21. The method according to claim 19, wherein the primary antibody is labeled, and wherein removing non-human in vivo bioreactor cells comprises utilizing the label to remove, from the elutriation fraction, elutriated xenomixture, or xenomixture, complexes comprising primary antibody and a non-human in vivo bioreactor cell.

22. The method according to claim 20 or claim 21, wherein the label comprises an affinity tag.

23. The method according to claim 20 or claim 21, wherein the label is magnetically responsive.

24. The method according to claim 23, wherein the label comprises a magnetic bead.

25. The method according to any one of claims 19 to 24, wherein the primary antibody is a pan-non-human in vivo bioreactor antibody.

26. The method according to claim 25, wherein the pan-non-human in vivo bioreactor antibody is an anti-histocompatibility antigen antibody.

27. The method according to claim 26, wherein the non-human in vivo bioreactor is a rat in vivo bioreactor.

28. The method according to claim 27, wherein the anti-histocompatibility antigen antibody is an anti-RT1-region, class I (A) (RT1A) antibody.

29. The method according to claim 28, wherein the anti-RT1A antibody competes for binding to RT1A with an antibody comprising:
a variable heavy chain (V_{H}) polypeptide comprising:
a V_{H} CDR1 comprising the amino acid sequence GDSITSGY (SEQ ID NO:1),
a V_{H} CDR2 comprising the amino acid sequence ISYSGST (SEQ ID NO:2), and
a V_{H} CDR3 comprising the amino acid sequence ASHSHWYFDV (SEQ ID NO:3), and
a variable light chain (V_{L}) polypeptide comprising:
a V_{L} CDR1 comprising the amino acid sequence QDISNY (SEQ ID NO:4),
a V_{L} CDR2 comprising the amino acid sequence YTS (SEQ ID NO:5), and
a V_{L} CDR3 comprising the amino acid sequence QQGNTLPWT (SEQ ID NO:6),
wherein CDRs are defined according to IMGT.

30. The method according to claim 28, wherein the anti-RT1A antibody comprises:
a variable heavy chain (V_{H}) polypeptide comprising
a V_{H} CDR1 comprising the amino acid sequence GDSITSGY (SEQ ID NO:1),
a V_{H} CDR2 comprising the amino acid sequence ISYSGST (SEQ ID NO:2), and
a V_{H} CDR3 comprising the amino acid sequence ASHSHWYFDV (SEQ ID NO:3), and TT comprising
a V_{L} CDR1 comprising the amino acid sequence QDISNY (SEQ ID NO:4),
a V_{L} CDR2 comprising the amino acid sequence YTS (SEQ ID NO:5), and
a V_{L} CDR3 comprising the amino acid sequence QQGNTLPWT (SEQ ID NO:6).

31. The method according to claim 29 or claim 30, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:15; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:18.

32. The method according to any one of claims 1 to 31, wherein the method does not comprise a step of centrifugal sedimentation to enrich for expanded human hepatocytes.

33. The method according to claim 32, wherein the isolated expanded human hepatocytes exhibit improved cell fitness as compared to a comparable human hepatocyte population isolated using centrifugal sedimentation.

34. The method according to any one of claims 1 to 33, wherein the isolated expanded human hepatocytes exhibit equivalent or improved cell fitness as compared to the human hepatocytes introduced into the liver of a non-human in vivo bioreactor.

35. The method according to any one of claims 32 to 34, wherein the isolated expanded human hepatocytes exhibit equivalent or improved cell fitness as compared to a comparable previously cryopreserved, freshly thawed human cadaveric hepatocyte population.

36. The method according to any one of claims 33 to 35, wherein the improved cell fitness is measured by an assay for attachment efficiency, ammonia detoxification, human albumin expression, A1AT expression, CYP3A4, or any combination thereof.

37. The method according to claim 36, wherein the improved cell fitness is measured by an in vivo human albumin assay.

38. The method according to any one of claims 1 to 37, comprising:
introducing human hepatocytes into the livers of a plurality of non-human in vivo bioreactors;
expanding the human hepatocytes in the livers of the non-human in vivo bioreactors;
collecting hepatocytes from the livers of the non-human in vivo bioreactors, wherein the collected hepatocytes comprise a xenomixture of expanded human hepatocytes and non-human in vivo bioreactor hepatocytes; and
subjecting the xenomixture to centrifugal elutriation under conditions sufficient to produce an elutriation fraction enriched for the expanded human hepatocytes and removing non-human in vivo bioreactor cells from the elutriation fraction via a negative selection process to produce isolated expanded human hepatocytes; or
removing non-human in vivo bioreactor cells from the xenomixture via a negative selection process and then subjecting the xenomixture to centrifugal elutriation under conditions sufficient to produce an elutriation fraction enriched for the expanded human hepatocytes to produce isolated expanded human hepatocytes.

39. The method according to claim 38, wherein the method comprises pooling the hepatocytes collected from the livers of the non-human in vivo bioreactors during the collecting, after the collecting, before the elutriation, during the elutriation, after the elutriation, before the negative selection process, during the negative selection process, or after the negative selection process.

40. The method according to any one of claims 1 to 39, wherein the human hepatocytes are derived from a single human donor.

41. Isolated expanded human hepatocytes produced according to the method of any one of claims 1 to 40.

42. The isolated expanded human hepatocytes of claim 41, wherein the isolated expanded human hepatocytes are cryopreserved.

43. The isolated expanded human hepatocytes of claim 41 or claim 42, wherein the isolated expanded human hepatocytes are derived from a single human donor.

44. A population of at least 1 billion of the isolated expanded human hepatocytes of any one of claims 41 to 43, optionally wherein the population is present in a single container.

45. A method comprising administering an effective amount of the isolated expanded human hepatocytes of any one of claim 41 or claim 44 to an individual in need thereof.

46. The method according to claim 45, wherein the individual in need thereof has acute liver failure, alcoholic liver disease, chronic liver disease, acute-on-chronic liver disease, liver fibrosis, liver cirrhosis, hepatic encephalopathy, hepatitis, or a combination thereof.

47. A method of enriching for human hepatocytes in a xenomixture, the method comprising:
subjecting a xenomixture comprising human hepatocytes and at least one type of non-human hepatocytes to centrifugal elutriation under conditions sufficient to produce an elutriation fraction enriched for the human hepatocytes.

48. The method according to claim 47, wherein the non-human hepatocytes are deficient for fumarylacetoacetate hydrolase (Fah).

49. The method according to claim 47 or claim 48, wherein the xenomixture comprises rodent hepatocytes.

50. The method according to claim 49, wherein the xenomixture comprises rat hepatocytes.

51. The method according to claim 49 or claim 50, wherein the rodent hepatocytes are deficient for interleukin 2 receptor subunit gamma (IL2rg), a recombination activating gene 1 (RAG1), a recombination activating gene 2 (RAG2), or a combination thereof.

52. A method of enriching for human hepatocytes in a xenomixture, the method comprising subjecting a xenomixture comprising human hepatocytes and non-human hepatocytes to an antibody-based negative selection process.

53. The method according to claim 52, wherein the xenomixture is produced from the liver of a in vivo bioreactor comprising the human hepatocytes and non-human hepatocytes.

54. The method according to claim 52 or claim 53, wherein the antibody-based negative selection process comprises:
contacting the xenomixture with a primary antibody specific for the non-human hepatocytes under conditions sufficient for specific binding of the primary antibody to the non-human hepatocytes; and
removing the non-human hepatocytes from the xenomixture utilizing the primary antibody.

55. The method according to claim 54, wherein removing the non-human hepatocytes from the xenomixture utilizing the primary antibody comprises contacting the antibody with a labeled secondary antibody under conditions sufficient for binding of the secondary antibody to the primary antibody, and utilizing the label of the labeled secondary antibody to remove from the xenomixture complexes comprising the labeled secondary antibody, the primary antibody, and the non-human hepatocyte.

56. The method according to claim 54, wherein the primary antibody is labeled, and wherein removing the non-human hepatocytes from the xenomixture comprises utilizing the label to remove from the xenomixture complexes comprising the primary antibody and the non-human hepatocyte.

57. The method according to claim 55 or claim 56, wherein the label comprises an affinity tag.

58. The method according to any one of claims 55 to 57, wherein the label is magnetically responsive.

59. The method according to claim 58, wherein the label comprises a magnetic bead.

60. The method according to any one of claims 54 to 59, wherein the antibody specific for the non-human hepatocytes is a pan-non-human antibody.

61. The method according to claim 60, wherein the pan-non-human antibody is an anti-histocompatibility antigen antibody.

62. The method according to claim 61, wherein the non-human hepatocytes are rat hepatocytes.

63. The method according to claim 62, wherein the anti-histocompatibility antigen antibody is an anti-RT1-region, class I (A) (RT1A) antibody.

64. The method according to claim 63, wherein the anti-RT1A antibody competes for binding to RT1A with an antibody comprising:
a variable heavy chain (V_{H}) polypeptide comprising:
a V_{H} CDR1 comprising the amino acid sequence GDSITSGY (SEQ ID NO:1),
a V_{H} CDR2 comprising the amino acid sequence ISYSGST (SEQ ID NO:2), and
a V_{H} CDR3 comprising the amino acid sequence ASHSHWYFDV (SEQ ID NO:3), and
a variable light chain (V_{L}) polypeptide comprising:
a V_{L} CDR1 comprising the amino acid sequence QDISNY (SEQ ID NO:4),
a V_{L} CDR2 comprising the amino acid sequence YTS (SEQ ID NO:5), and
a V_{L} CDR3 comprising the amino acid sequence QQGNTLPWT (SEQ ID NO:6),
wherein CDRs are defined according to IMGT.

65. The method according to claim 63, wherein the anti-RT1A antibody comprises:
a variable heavy chain (V_{H}) polypeptide comprising:
a V_{H} CDR1 comprising the amino acid sequence GDSITSGY (SEQ ID NO:1),
a V_{H} CDR2 comprising the amino acid sequence ISYSGST (SEQ ID NO:2), and
a V_{H} CDR3 comprising the amino acid sequence ASHSHWYFDV (SEQ ID NO:3), and
a variable light chain (V_{L}) polypeptide comprising:
a V_{L} CDR1 comprising the amino acid sequence QDISNY (SEQ ID NO:4),
a V_{L} CDR2 comprising the amino acid sequence YTS (SEQ ID NO:5), and
a V_{L} CDR3 comprising the amino acid sequence QQGNTLPWT (SEQ ID NO:6).

66. The method according to claim 64 or claim 65, wherein the antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:15; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:18.

67. A composition comprising a cell population derived from a xenomixture, the xenomixture comprising dissociated human hepatocytes and at least one type of non-human hepatocytes, the cell population comprising at least 60% human hepatocytes, optionally wherein the composition comprises 40% or less non-human hepatocytes.

68. A composition comprising isolated expanded human hepatocytes produced according to the method of any one of claims 1 to 40.

69. A pharmaceutical preparation suitable for delivery to a human subject, the pharmaceutical preparation comprising the composition of claim 67 or claim 68 and at least 1 billion of the human hepatocytes.

70. The pharmaceutical preparation according to claim 69, wherein the at least 1 billion hepatocytes are derived from a single human donor.

71. An isolated expanded population of human hepatocytes, wherein the population:
is expanded from an initial population of human hepatocytes obtained from a human liver or a portion thereof;
is isolated following expansion by a process that excludes centrifugal sedimentation; and
displays improved cell fitness, as measured by one or more potency assays, as compared to a comparable human hepatocyte population isolated using centrifugal sedimentation.

72. The population of human hepatocytes of claim 71, wherein the isolated expanded population of human hepatocytes displays equivalent or improved cell fitness as compared to the initial population of human hepatocytes, as measured by one or more potency assays.

73. The population of human hepatocytes of claim 71 or claim 72, wherein the improved cell fitness is measured by an assay for attachment efficiency, ammonia detoxification, human albumin expression, A1AT expression, CYP3A4, or any combination thereof.

74. An isolated expanded population of human hepatocytes, wherein the human hepatocytes exhibit:
in vivo human albumin expression levels greater than or equal to freshly isolated and/or cryopreserved cadaveric hepatocytes;
a reduced amount of immune cells and/or inflammatory cytokines as compared to freshly isolated and/or cryopreserved cadaveric hepatocytes, optionally wherein the inflammatory cytokines are selected from IL-1-beta, IL-6, TNF-alpha, and TGF-beta and/or the immune cells are selected from IL-1-beta-, IL-6-TNF-alpha-, and TGF-beta-expressing immune cells; or
a combination thereof.

75. An isolated nucleic acid comprising one or more coding sequences encoding a variable heavy chain (V_{H}) polypeptide and/or a variable light chain (V_{L}) polypeptide of an anti-RT1A antibody, wherein the one or more coding sequences are mammalian codon optimized.

76. The isolated nucleic acid of claim 75, wherein the anti-RT1A antibody comprises or competes with for binding to RT1A with an antibody comprising:
a variable heavy chain (V_{H}) polypeptide comprising:
a V_{H} CDR1 comprising the amino acid sequence GDSITSGY (SEQ ID NO:1),
a V_{H} CDR2 comprising the amino acid sequence ISYSGST (SEQ ID NO:2), and
a V_{H} CDR3 comprising the amino acid sequence ASHSHWYFDV (SEQ ID NO:3), and
a variable light chain (V_{L}) polypeptide comprising:
a V_{L} CDR1 comprising the amino acid sequence QDISNY (SEQ ID NO:4),
a V_{L} CDR2 comprising the amino acid sequence YTS (SEQ ID NO:5), and
a V_{L} CDR3 comprising the amino acid sequence QQGNTLPWT (SEQ ID NO:6).

77. The isolated nucleic acid of claim 75 or claim 76, wherein the anti-RT1A antibody comprises:
a variable heavy chain (V_{H}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:15; and
a variable light chain (V_{L}) polypeptide comprising an amino acid sequence having 70% or greater, 75% or greater, 80% or greater, 85% or greater, 90% or greater, 95% or greater, 98% or greater, 99% or greater, or 100% identity to the amino acid sequence set forth in SEQ ID NO:18.

78. The isolated nucleic acid of any one of claims 75 to 77, wherein the one or more coding sequences comprise:
a sequence having 70% or greater, 75% or greater, 80% or greater, 85% or greater, 90% or greater, 95% or greater, 98% or greater, 99% or greater, or 100% identity to the nucleic acid sequence set forth in SEQ ID NO:14;
a sequence having 70% or greater, 75% or greater, 80% or greater, 85% or greater, 90% or greater, 95% or greater, 98% or greater, 99% or greater, or 100% identity to the nucleic acid sequence set forth in SEQ ID NO:17; or
a combination thereof.

79. An expression vector comprising the isolated nucleic acid of any one of claims 75 to 78.

80. An isolated expanded population of human hepatocytes having a gene signature comprising:
elevated expression of two or more, three or more, or four or more genes selected from Table 4;
reduced expression of two or more, three or more, or four or more genes selected from Table 5; or
elevated expression of at least one gene selected from Table 4 and reduced expression of at least one gene selected from Table 5,
optionally wherein the elevated and/or reduced expression is determined by comparison to corresponding gene expression in a reference primary human hepatocyte population.

81. The isolated expanded population of human hepatocytes of claim 80, wherein the gene signature comprises:
elevated expression of two or more, three or more, or four or more genes selected from the group consisting of: GPC3, AKR1B10, FXYD2, PEG10, CYP7A1, and NQO1;
reduced expression of two or more, three or more, or four or more genes selected from the group consisting of: C9, SAA1, SAA2, CRP, NNMT, SPINK1, PLA2G2A, and ORM1; or
elevated expression of at least one gene selected from the group consisting of GPC3, AKR1B10, FXYD2, PEG10, CYP7A1, and NQO1 and reduced expression of at least one gene selected from the group consisting of C9, SAA1, SAA2, CRP, NNMT, SPINK1, PLA2G2A, and ORM1.

82. The isolated expanded population of human hepatocytes of claim 80 or claim 81, wherein the elevated expression comprises an at least 2-fold elevation, as compared to corresponding expression in primary human hepatocytes, of each of the elevated genes of the gene signature and the reduced expression comprises an at least 2-fold reduction, as compared to corresponding expression in primary human hepatocytes, of each of the reduced genes of the gene signature.

83. The isolated expanded population of human hepatocytes of any one of claims 80 to 82, wherein the isolated expanded human hepatocytes of the population are derived from a single human donor.

84. The isolated expanded population of human hepatocytes of any one of claims 80 to 83, wherein the population comprises at least 1 billion of the isolated expanded human hepatocytes, optionally wherein the population is present in a single container.

85. The isolated expanded population of human hepatocytes of any one of claims 80 to 84, wherein the population cryopreserved.

86. A pharmaceutical preparation suitable for delivery to a human subject, the pharmaceutical preparation comprising the isolated expanded population of human hepatocytes of any one of claims 80 to 85.

87. A method comprising administering an effective amount of the population of isolated expanded human hepatocytes of any one of claims 80 to 84 or pharmaceutical preparation claim 86 to an individual in need thereof.

88. The method according to claim 87, wherein the individual in need thereof has acute liver failure, alcoholic liver disease, chronic liver disease, acute-on-chronic liver disease, liver fibrosis, liver cirrhosis, hepatic encephalopathy, hepatitis, or a combination thereof.
